SEQUENCE LISTING (307 sequences)

SEQ ID NO: 1 - RNA, 72 nt, Artificial Sequence
  Synthetic
ggccucguggcgcaacgguagcgcgucugacuucagaucagaagguugcguguucaaaucacgucgggguca

SEQ ID NO: 2 - RNA, 83 nt, Artificial Sequence
  Synthetic
accaggauggccgagugguuaaggcguuggacuucagauccaauggacauauguccgcguggguucgaaccccacuccuggua

SEQ ID NO: 3 - RNA, 83 nt, Artificial Sequence
  Synthetic
accgggauggccgagugguuaaggcguuggacuucagauccaaugggcuggugcccgcguggguucgaaccccacucucggua

SEQ ID NO: 4 - RNA, 83 nt, Artificial Sequence
  Synthetic
accagaauggccgagugguuaaggcguuggacuucagauccaauggauucauauccgcguggguucgaaccccacuucuggua

SEQ ID NO: 5 - RNA, 72 nt, Artificial Sequence
  Synthetic
gcguuggugguauagugguuagcauagcugccuucaaagcaguugacccggguucgauucccggccaacgca

SEQ ID NO: 6 - RNA, 72 nt, Artificial Sequence
  Synthetic
gcguuggugguauaguggugagcauagcugccuucaaagcaguugacccggguucgauucccggccaacgca

SEQ ID NO: 7 - RNA, 72 nt, Artificial Sequence
  Synthetic
gcguuggugguauagugguaagcauagcugccuucaaagcaguugacccggguucgauucccggccaacgca

SEQ ID NO: 8 - RNA, 73 nt, Artificial Sequence
  Synthetic
ggcucuguggcgcaauggauagcgcauuggacuucaaauucaaagguuguggguucgagucccaccagagucg

SEQ ID NO: 9 - RNA, 72 nt, Artificial Sequence
  Synthetic
gguuccaugguguaaugguaagcacucuggacucuaaauccagcgauccgaguucgagucucgguggaaccu

SEQ ID NO: 10 - RNA, 72 nt, Artificial Sequence
  Synthetic
ggccccaugguguaaugguuagcacucuggacucuaaauccagcgauccgaguucaaaucucggugggaccu

SEQ ID NO: 11 - DNA, 72 bp, Artificial Sequence
  Synthetic
ggcctcgtggcgcaacggtagcgcgtctgacttcagatcagaaggttgcgtgttcaaatcacgtcggggtca

SEQ ID NO: 12 - DNA, 72 bp, Artificial Sequence
  Synthetic
gacctcgtggcgcaatggtagcgcgtctgacttcagatcagaaggttgcgtgttcaagtcacgtcggggtca

SEQ ID NO: 13 - DNA, 72 bp, Artificial Sequence
  Synthetic
gacctcgtggcgcaacggtagcgcgtctgacttcagatcagaaggttgcgtgttcaaatcacgtcggggtca

SEQ ID NO: 14 - DNA, 72 bp, Artificial Sequence
  Synthetic
gacctcgtggcgcaacggtagcgcgtctgacttcagatcagaaggctgcgtgttcgaatcacgtcggggtca

SEQ ID NO: 15 - DNA, 72 bp, Artificial Sequence
  Synthetic
gacctcgtggcgcaacggcagcgcgtctgacttcagatcagaaggttgcgtgttcaaatcacgtcggggtca

SEQ ID NO: 16 - DNA, 72 bp, Artificial Sequence
  Synthetic
ggcctcgtggcgcaacggtagcgcgtctgactctagatcagaaggttgcgtgttcaaatcacgtcggggtca

SEQ ID NO: 17 - DNA, 72 bp, Artificial Sequence
  Synthetic
gacctcgtggcgcaatggtagcgcgtctgactctagatcagaaggttgcgtgttcaagtcacgtcggggtca

SEQ ID NO: 18 - DNA, 72 bp, Artificial Sequence
  Synthetic
gacctcgtggcgcaacggtagcgcgtctgactctagatcagaaggttgcgtgttcaaatcacgtcggggtca

SEQ ID NO: 19 - DNA, 72 bp, Artificial Sequence
  Synthetic
gacctcgtggcgcaacggtagcgcgtctgactctagatcagaaggctgcgtgttcgaatcacgtcggggtca

SEQ ID NO: 20 - DNA, 72 bp, Artificial Sequence
  Synthetic
gacctcgtggcgcaacggcagcgcgtctgactctagatcagaaggttgcgtgttcaaatcacgtcggggtca

SEQ ID NO: 21 - DNA, 72 bp, Artificial Sequence
  Synthetic
gcgttggtggtatagtggttagcatagctgccttcaaagcagttgacccgggttcgattcccggccaacgca

SEQ ID NO: 22 - DNA, 72 bp, Artificial Sequence
  Synthetic
gcgttggtggtatagtggtgagcatagctgccttcaaagcagttgacccgggttcgattcccggccaacgca

SEQ ID NO: 23 - DNA, 72 bp, Artificial Sequence
  Synthetic
gcgttggtggtatagtggtaagcatagctgccttcaaagcagttgacccgggttcgattcccggccaacgca

SEQ ID NO: 24 - DNA, 73 bp, Artificial Sequence
  Synthetic
gggccagtggcgcaatggataacgcgtctgacttcagatcagaagattctaggttcgactcctggctggctcg

SEQ ID NO: 25 - DNA, 73 bp, Artificial Sequence
  Synthetic
ggccgcgtggcctaatggataaggcgtctgatttcagatcagaagattgagggttcgagtcccttcgtggtcg

SEQ ID NO: 26 - DNA, 73 bp, Artificial Sequence
  Synthetic
gacccagtggcctaatggataaggcatcagccttcagagctggggattgtgggttcgagtcccatctgggtcg

SEQ ID NO: 27 - DNA, 73 bp, Artificial Sequence
  Synthetic
gccccagtggcctaatggataaggcactggccttcaaagccagggattgtgggttcgagtcccacctggggta

SEQ ID NO: 28 - DNA, 73 bp, Artificial Sequence
  Synthetic
gccccagtggcctaatggataaggcactggccttcaaagccagggattgtgggttcgagtcccacctggggtg

SEQ ID NO: 29 - DNA, 73 bp, Artificial Sequence
  Synthetic
gccccggtggcctaatggataaggcattggccttcaaagccagggattgtgggttcgagtcccacccggggta

SEQ ID NO: 30 - DNA, 73 bp, Artificial Sequence
  Synthetic
gccccagtggcctaatggataaggcattggccttcaaagccagggattgtgggttcgagtcccatctggggtg

SEQ ID NO: 31 - DNA, 73 bp, Artificial Sequence
  Synthetic
gccccagtggcctgatggataaggtactggccttcaaagccagggattgtgggttcgagttccacctggggta

SEQ ID NO: 32 - DNA, 73 bp, Artificial Sequence
  Synthetic
ggccgcgtggcctaatggataaggcgtctgacttcagatcagaagattgcaggttcgagtcctgccgcggtcg

SEQ ID NO: 33 - DNA, 73 bp, Artificial Sequence
  Synthetic
gaccacgtggcctaatggataaggcgtctgacttcagatcagaagattgagggttcgaatccctccgtggtta

SEQ ID NO: 34 - DNA, 73 bp, Artificial Sequence
  Synthetic
gaccgcgtggcctaatggataaggcgtctgacttcagatcagaagattgagggttcgagtcccttcgtggtcg

SEQ ID NO: 35 - DNA, 73 bp, Artificial Sequence
  Synthetic
gaccacgtggcctaatggataaggcgtctgacttcagatcagaagattgagggttcgaatcccttcgtggtta

SEQ ID NO: 36 - DNA, 73 bp, Artificial Sequence
  Synthetic
gaccacgtggcctaatggataaggcgtctgacttcagatcagaagattgagggttcgaatcccttcgtggttg

SEQ ID NO: 37 - DNA, 73 bp, Artificial Sequence
  Synthetic
ggccgtgtggcctaatggataaggcgtctgacttcagatcaaaagattgcaggtttgagttctgccacggtcg

SEQ ID NO: 38 - DNA, 85 bp, Artificial Sequence
  Synthetic
ggctccgtggcgcaatggatagcgcattggacttcaagaggctgaaggcattcaaaggttccgggttcgagtcccggcggagtcg

SEQ ID NO: 39 - DNA, 73 bp, Artificial Sequence
  Synthetic
ggctccgtggcgcaatggatagcgcattggacttcaaattcaaaggttccgggttcgagtcccggcggagtcg

SEQ ID NO: 40 - DNA, 88 bp, Artificial Sequence
  Synthetic
ggctctgtggcgcaatggatagcgcattggacttcaagtgacgaatagagcaattcaaaggttgtgggttcgaatcccaccagagtcg

SEQ ID NO: 41 - DNA, 73 bp, Artificial Sequence
  Synthetic
ggctctgtggcgcaatggatagcgcattggacttcaaattcaaaggttgtgggttcgaatcccaccagagtcg

SEQ ID NO: 42 - DNA, 91 bp, Artificial Sequence
  Synthetic
ggctctgtggcgcaatggatagcgcattggacttcaagctgagcctagtgtggtcattcaaaggttgtgggttcgagtcccaccagagtcg

SEQ ID NO: 43 - DNA, 73 bp, Artificial Sequence
  Synthetic
ggctctgtggcgcaatggatagcgcattggacttcaaattcaaaggttgtgggttcgagtcccaccagagtcg

SEQ ID NO: 44 - DNA, 86 bp, Artificial Sequence
  Synthetic
ggctctgtggcgcaatggatagcgcattggacttcaagatagttagagaaattcaaaggttgtgggttcgagtcccaccagagtcg

SEQ ID NO: 45 - DNA, 74 bp, Artificial Sequence
  Synthetic
gtctctgtggcgcaatggacgagcgcgctggacttcaaatccagaggttccgggttcgagtcccggcagagatg

SEQ ID NO: 46 - DNA, 73 bp, Artificial Sequence
  Synthetic
ggctctgtggcgcaatggatagcgcattggacttcaaattcaaaggttgcgggttcgagtccctccagagtcg

SEQ ID NO: 47 - DNA, 73 bp, Artificial Sequence
  Synthetic
ggttccatggtgtaatggtaagcaccctggactctaaatccagcaaccagagttccagtctcagcgtggacct

SEQ ID NO: 48 - DNA, 72 bp, Artificial Sequence
  Synthetic
ggtagtgtagtctactggttaaacgcttgggctctaacattaacgtcctgggttcaaatcccagctttgtca

SEQ ID NO: 49 - DNA, 72 bp, Artificial Sequence
  Synthetic
ggttccatggtgtaatggttagcactctggactctaaatccagcgatccgagttcaagtctcggtggaacct

SEQ ID NO: 50 - DNA, 72 bp, Artificial Sequence
  Synthetic
ggttccatggtgtaatggtgagcactctggactctaaatccagcgatccgagttcgagtctcggtggaacct

SEQ ID NO: 51 - DNA, 72 bp, Artificial Sequence
  Synthetic
ggttccatggtgtaatggtaagcactctggactctaaatccagcgatccgagttcgagtctcggtggaacct

SEQ ID NO: 52 - DNA, 72 bp, Artificial Sequence
  Synthetic
ggttccatggtgtaatggttagcactctggactctaaatccggtaatccgagttcaaatctcggtggaacct

SEQ ID NO: 53 - DNA, 72 bp, Artificial Sequence
  Synthetic
ggccccatggtgtaatggtcagcactctggactctaaatccagcgatccgagttcaaatctcggtgggaccc

SEQ ID NO: 54 - DNA, 72 bp, Artificial Sequence
  Synthetic
ggttccatggtgtaatggtaagcactctggactctaaatccagccatctgagttcgagtctctgtggaacct

SEQ ID NO: 55 - DNA, 72 bp, Artificial Sequence
  Synthetic
ggttccatggtgtaatggtgagcactttggactctaaatacagtgatcagagttcaagtctcactgggacct

SEQ ID NO: 56 - DNA, 72 bp, Artificial Sequence
  Synthetic
ggttccatgggttaatggtgagcaccctggactctaaatcaagcgatccgagttcaaatctcggtggtacct

SEQ ID NO: 57 - DNA, 68 bp, Artificial Sequence
  Synthetic
gtttccatggtgtaatggtgagcactctggactctaaatccagaaatacattcaaagaattaagaaca

SEQ ID NO: 58 - DNA, 72 bp, Artificial Sequence
  Synthetic
ggtcccatggtgtaatggttagcactctggactctaaatccagcgatccgagttcaaatctcggtgggacct

SEQ ID NO: 59 - DNA, 72 bp, Artificial Sequence
  Synthetic
ggtcccatggtgtaatggttagcactctggactctaaatccagcaatccgagttcgaatctcggtgggacct

SEQ ID NO: 60 - DNA, 72 bp, Artificial Sequence
  Synthetic
ggccccatggtgtaatggttagcactctggactctaaatccagcgatccgagttcaaatctcggtgggacct

SEQ ID NO: 61 - DNA, 72 bp, Artificial Sequence
  Synthetic
ggtcccatggtgtaatggttagcactctgggctctaaatccagcaatccgagttcgaatcttggtgggacct

SEQ ID NO: 62 - DNA, 73 bp, Artificial Sequence
  Synthetic
ggctgtgtacctcagtgggcaagggtatggactctaaagccagactatttgggttcaaatcccagcttggcct

SEQ ID NO: 63 - DNA, 72 bp, Artificial Sequence
  Synthetic
taggacatggtgtgataggtagcatggagaattttagattctcaggggtaggttcaattcctacagttctag

SEQ ID NO: 64 - DNA, 72 bp, Artificial Sequence
  Synthetic
taggacgtggtgtgataggtagcatggggaattttagattctcaggggtgggttcaattcctatagttctag

SEQ ID NO: 65 - DNA, 72 bp, Artificial Sequence
  Synthetic
taggacgtggtgtagtaggtagcatggagaatgttaaattctcaggggtaggttcaattcctatagttctag

SEQ ID NO: 66 - DNA, 75 bp, Artificial Sequence
  Synthetic
tctaggatgtggtgtgataggtagcatggagaattttagattctcaggggtaggttcaattcctatattctagaa

SEQ ID NO: 67 - DNA, 72 bp, Artificial Sequence
  Synthetic
taggacgtggtgtgataggtagcatggagaattttagattctcagggatgggttcaattcctatagtcctag

SEQ ID NO: 68 - DNA, 72 bp, Artificial Sequence
  Synthetic
taggacgtggtgtgataggtagcacggagaattttagattctcagggatgggttcaattcctgtagttctag

SEQ ID NO: 69 - DNA, 72 bp, Artificial Sequence
  Synthetic
ggttccatggtgtaatggttagcactctggactttaaatccagcgatccgagttcaaatctcggtggaacct

SEQ ID NO: 70 - DNA, 72 bp, Artificial Sequence
  Synthetic
ggttccatggtgtaatggttagcactctggactttaaatccagcgatccgagttcaagtctcggtggaacct

SEQ ID NO: 71 - DNA, 72 bp, Artificial Sequence
  Synthetic
ggttccatggtgtaatggtgagcactctggactttaaatccagcgatccgagttcgagtctcggtggaacct

SEQ ID NO: 72 - DNA, 72 bp, Artificial Sequence
  Synthetic
ggttccatggtgtaatggtaagcactctggactttaaatccagcgatccgagttcgagtctcggtggaacct

SEQ ID NO: 73 - DNA, 72 bp, Artificial Sequence
  Synthetic
ggttccatggtgtaatggttagcactctggactttaaatccggtaatccgagttcaaatctcggtggaacct

SEQ ID NO: 74 - DNA, 72 bp, Artificial Sequence
  Synthetic
ggccccatggtgtaatggtcagcactctggactttaaatccagcgatccgagttcaaatctcggtgggaccc

SEQ ID NO: 75 - DNA, 72 bp, Artificial Sequence
  Synthetic
ggttccatggtgtaatggtaagcactctggactttaaatccagccatctgagttcgagtctctgtggaacct

SEQ ID NO: 76 - DNA, 72 bp, Artificial Sequence
  Synthetic
ggttccatggtgtaatggtgagcactttggactttaaatacagtgatcagagttcaagtctcactgggacct

SEQ ID NO: 77 - DNA, 72 bp, Artificial Sequence
  Synthetic
ggttccatgggttaatggtgagcaccctggactttaaatcaagcgatccgagttcaaatctcggtggtacct

SEQ ID NO: 78 - DNA, 68 bp, Artificial Sequence
  Synthetic
gtttccatggtgtaatggtgagcactctggactttaaatccagaaatacattcaaagaattaagaaca

SEQ ID NO: 79 - DNA, 72 bp, Artificial Sequence
  Synthetic
ggtcccatggtgtaatggttagcactctggactttaaatccagcgatccgagttcaaatctcggtgggacct

SEQ ID NO: 80 - DNA, 72 bp, Artificial Sequence
  Synthetic
ggtcccatggtgtaatggttagcactctggactttaaatccagcaatccgagttcgaatctcggtgggacct

SEQ ID NO: 81 - DNA, 72 bp, Artificial Sequence
  Synthetic
ggccccatggtgtaatggttagcactctggactttaaatccagcgatccgagttcaaatctcggtgggacct

SEQ ID NO: 82 - DNA, 72 bp, Artificial Sequence
  Synthetic
ggtcccatggtgtaatggttagcactctgggctttaaatccagcaatccgagttcgaatcttggtgggacct

SEQ ID NO: 83 - DNA, 73 bp, Artificial Sequence
  Synthetic
ggctgtgtacctcagtgggcaagggtatggactttaaagccagactatttgggttcaaatcccagcttggcct

SEQ ID NO: 84 - DNA, 74 bp, Artificial Sequence
  Synthetic
ggtacagtgttaaaggggagaaaaattgctgactttaaatacagtagacctaggtttgaatcctggctttacca

SEQ ID NO: 85 - DNA, 72 bp, Artificial Sequence
  Synthetic
tccctggtggtctagtggttaggattcggcgctttaaccgccgcggcccgggttcgattcccggtcagggaa

SEQ ID NO: 86 - DNA, 72 bp, Artificial Sequence
  Synthetic
tccctggtggtctagtggttaggattcggcgctttaaccgccgcggcccgggttcgattcccggtcaggaaa

SEQ ID NO: 87 - DNA, 72 bp, Artificial Sequence
  Synthetic
tcccatatggtctagcggttaggattcctggttttaacccaggtggcccgggttcgactcccggtatgggaa

SEQ ID NO: 88 - DNA, 72 bp, Artificial Sequence
  Synthetic
tcccacatggtctagcggttaggattcctggttttaacccaggcggcccgggttcgactcccggtgtgggaa

SEQ ID NO: 89 - DNA, 72 bp, Artificial Sequence
  Synthetic
tccctggtggtctagtggctaggattcggcgctttaaccgccgcggcccgggttcgattcccggccagggaa

SEQ ID NO: 90 - DNA, 72 bp, Artificial Sequence
  Synthetic
tccctggtggtctagtggctaggattcggcgctttaaccgccgcggcccgggttcgattcccggtcagggaa

SEQ ID NO: 91 - DNA, 72 bp, Artificial Sequence
  Synthetic
tccctggtggtctagtggttaggattcggcgctctaaccgccgcggcccgggttcgattcccggtcagggaa

SEQ ID NO: 92 - DNA, 72 bp, Artificial Sequence
  Synthetic
tcccatatggtctagcggttaggattcctggttctaacccaggtggcccgggttcgactcccggtatgggaa

SEQ ID NO: 93 - DNA, 73 bp, Artificial Sequence
  Synthetic
tccgtggtggtctagtggctaggattcggcgctctaaccgcctgcagctcgagttcgattcctggtcagggaa

SEQ ID NO: 94 - DNA, 72 bp, Artificial Sequence
  Synthetic
tcccacatggtctagcggttaggattcctggttctaacccaggcggcccgggttcgactcccggtgtgggaa

SEQ ID NO: 95 - DNA, 72 bp, Artificial Sequence
  Synthetic
tccctggtggtctagtggctaggattcggcgctctaaccgccgcggcccgggttcgattcccggccagggaa

SEQ ID NO: 96 - DNA, 72 bp, Artificial Sequence
  Synthetic
tccctggtggtctagtggctaggattcggcgctctaaccgccgcggcccgggttcgattcccggtcagggaa

SEQ ID NO: 97 - DNA, 73 bp, Artificial Sequence
  Synthetic
tccctggtggtctagtggctaggattcggcgctctaaccgcctgcagctcgagttcgattcctggtcagggaa

SEQ ID NO: 98 - DNA, 91 bp, Artificial Sequence
  Synthetic
ccttcgatagctcagttggtagagcggaggactttagttggctgtgtccttagacatccttaggtcgctggttcgaatccggctcgaagga

SEQ ID NO: 99 - DNA, 73 bp, Artificial Sequence
  Synthetic
ccttcgatagctcagttggtagagcggaggactttagatccttaggtcgctggttcgaatccggctcgaagga

SEQ ID NO: 100 - DNA, 73 bp, Artificial Sequence
  Synthetic
gggggtatagctcagggctagagctttttgactttagagcaagaggtccctggttcaaatccaggttctccct

SEQ ID NO: 101 - DNA, 61 bp, Artificial Sequence
  Synthetic
tatagctcagtggtagagcatttaactttagatcaagaggtccctggatcaactctgggtg

SEQ ID NO: 102 - DNA, 73 bp, Artificial Sequence
  Synthetic
gtcagtgttgcacaacggttaagtgaagaggctttaaacccagactggatgggttcaattcccatctctgccg

SEQ ID NO: 103 - DNA, 89 bp, Artificial Sequence
  Synthetic
ccttcgatagctcagttggtagagcggaggactttagtggatagggcgtggcaatccttaggtcgctggttcgattccggctcgaagga

SEQ ID NO: 104 - DNA, 73 bp, Artificial Sequence
  Synthetic
ccttcgatagctcagttggtagagcggaggactttagatccttaggtcgctggttcgattccggctcgaagga

SEQ ID NO: 105 - DNA, 89 bp, Artificial Sequence
  Synthetic
ccttcgatagctcagttggtagagcggaggactttaggctcattaagcaaggtatccttaggtcgctggttcgaatccggctcggagga

SEQ ID NO: 106 - DNA, 73 bp, Artificial Sequence
  Synthetic
ccttcgatagctcagttggtagagcggaggactttagatccttaggtcgctggttcgaatccggctcggagga

SEQ ID NO: 107 - DNA, 94 bp, Artificial Sequence
  Synthetic
ccttcgatagctcagctggtagagcggaggactttagattgtatagacatttgcggacatccttaggtcgctggttcgattccagctcgaagga

SEQ ID NO: 108 - DNA, 73 bp, Artificial Sequence
  Synthetic
ccttcgatagctcagctggtagagcggaggactttagatccttaggtcgctggttcgattccagctcgaagga

SEQ ID NO: 109 - DNA, 93 bp, Artificial Sequence
  Synthetic
ccttcgatagctcagctggtagagcggaggactttagctacttcctcagcaggagacatccttaggtcgctggttcgattccggctcgaagga

SEQ ID NO: 110 - DNA, 73 bp, Artificial Sequence
  Synthetic
ccttcgatagctcagctggtagagcggaggactttagatccttaggtcgctggttcgattccggctcgaagga

SEQ ID NO: 111 - DNA, 89 bp, Artificial Sequence
  Synthetic
ccttcgatagctcagctggtagagcggaggactttaggcgcgcgcccgtggccatccttaggtcgctggttcgattccggctcgaagga

SEQ ID NO: 112 - DNA, 73 bp, Artificial Sequence; identical to SEQ ID NO: 110
  Synthetic

SEQ ID NO: 113 - DNA, 94 bp, Artificial Sequence
  Synthetic
ccttcgatagctcagctggtagagcggaggactttagcctgtagaaacatttgtggacatccttaggtcgctggttcgattccggctcgaagga

SEQ ID NO: 114 - DNA, 73 bp, Artificial Sequence; identical to SEQ ID NO: 110
  Synthetic

SEQ ID NO: 115 - DNA, 94 bp, Artificial Sequence
  Synthetic
ccttcgatagctcagctggtagagcggaggactttagattgtacagacatttgcggacatccttaggtcgctggttcgattccggctcgaagga

SEQ ID NO: 116 - DNA, 73 bp, Artificial Sequence; identical to SEQ ID NO: 110
  Synthetic

SEQ ID NO: 117 - DNA, 89 bp, Artificial Sequence
  Synthetic
ccttcgatagctcagctggtagagcggaggactttagtacttaatgtgtggtcatccttaggtcgctggttcgattccggctcgaagga

SEQ ID NO: 118 - DNA, 73 bp, Artificial Sequence; identical to SEQ ID NO: 110
  Synthetic

SEQ ID NO: 119 - DNA, 89 bp, Artificial Sequence
  Synthetic
ccttcgatagctcagctggtagagcggaggactttaggggtttgaatgtggtcatccttaggtcgctggttcgaatccggctcggagga

SEQ ID NO: 120 - DNA, 73 bp, Artificial Sequence
  Synthetic
ccttcgatagctcagctggtagagcggaggactttagatccttaggtcgctggttcgaatccggctcggagga

SEQ ID NO: 121 - DNA, 94 bp, Artificial Sequence
  Synthetic
ccttcgatagctcagctggtagagcggaggactttagactgcggaaacgtttgtggacatccttaggtcgctggttcaattccggctcgaagga

SEQ ID NO: 122 - DNA, 73 bp, Artificial Sequence
  Synthetic
ccttcgatagctcagctggtagagcggaggactttagatccttaggtcgctggttcaattccggctcgaagga

SEQ ID NO: 123 - DNA, 90 bp, Artificial Sequence
  Synthetic
ctttcgatagctcagttggtagagcggaggactttaggttcattaaactaaggcatccttaggtcgctggttcgaatccggctcgaagga

SEQ ID NO: 124 - DNA, 73 bp, Artificial Sequence
  Synthetic
ctttcgatagctcagttggtagagcggaggactttagatccttaggtcgctggttcgaatccggctcgaagga

SEQ ID NO: 125 - DNA, 88 bp, Artificial Sequence
  Synthetic
tcttcaatagctcagctggtagagcggaggactttaggtgcacgcccgtggccattcttaggtgctggtttgattccgacttggagag

SEQ ID NO: 126 - DNA, 66 bp, Artificial Sequence
  Synthetic
ggtaaaatggctgagtaagcattagactttaaatctaaagacagaggtcaaggcctcttttttcct

SEQ ID NO: 127 - DNA, 66 bp, Artificial Sequence
  Synthetic
ggtaaaatgactgagtaagcattagactctaaatctaaagacagaggtcaagacctctttttacca

SEQ ID NO: 128 - DNA, 66 bp, Artificial Sequence
  Synthetic
ggtaaaatggctgagtaagcattagactctaaatctaaagacagaggtcaaggcctctttttacca

SEQ ID NO: 129 - DNA, 91 bp, Artificial Sequence
  Synthetic
ccttcgatagctcagttggtagagcggaggactctagttggctgtgtccttagacatccttaggtcgctggttcgaatccggctcgaagga

SEQ ID NO: 130 - DNA, 73 bp, Artificial Sequence
  Synthetic
ccttcgatagctcagttggtagagcggaggactctagatccttaggtcgctggttcgaatccggctcgaagga

SEQ ID NO: 131 - DNA, 73 bp, Artificial Sequence
  Synthetic
gggggtatagctcagggctagagctttttgactctagagcaagaggtccctggttcaaatccaggttctccct

SEQ ID NO: 132 - DNA, 61 bp, Artificial Sequence
  Synthetic
tatagctcagtggtagagcatttaactctagatcaagaggtccctggatcaactctgggtg

SEQ ID NO: 133 - DNA, 73 bp, Artificial Sequence
  Synthetic
gtcagtgttgcacaacggttaagtgaagaggctctaaacccagactggatgggttcaattcccatctctgccg

SEQ ID NO: 134 - DNA, 89 bp, Artificial Sequence
  Synthetic
ccttcgatagctcagttggtagagcggaggactctagtggatagggcgtggcaatccttaggtcgctggttcgattccggctcgaagga

SEQ ID NO: 135 - DNA, 73 bp, Artificial Sequence
  Synthetic
ccttcgatagctcagttggtagagcggaggactctagatccttaggtcgctggttcgattccggctcgaagga

SEQ ID NO: 136 - DNA, 89 bp, Artificial Sequence
  Synthetic
ccttcgatagctcagttggtagagcggaggactctaggctcattaagcaaggtatccttaggtcgctggttcgaatccggctcggagga

SEQ ID NO: 137 - DNA, 73 bp, Artificial Sequence
  Synthetic
ccttcgatagctcagttggtagagcggaggactctagatccttaggtcgctggttcgaatccggctcggagga

SEQ ID NO: 138 - DNA, 94 bp, Artificial Sequence
  Synthetic
ccttcgatagctcagctggtagagcggaggactctagattgtatagacatttgcggacatccttaggtcgctggttcgattccagctcgaagga

SEQ ID NO: 139 - DNA, 73 bp, Artificial Sequence
  Synthetic
ccttcgatagctcagctggtagagcggaggactctagatccttaggtcgctggttcgattccagctcgaagga

SEQ ID NO: 140 - DNA, 93 bp, Artificial Sequence
  Synthetic
ccttcgatagctcagctggtagagcggaggactctagctacttcctcagcaggagacatccttaggtcgctggttcgattccggctcgaagga

SEQ ID NO: 141 - DNA, 73 bp, Artificial Sequence
  Synthetic
ccttcgatagctcagctggtagagcggaggactctagatccttaggtcgctggttcgattccggctcgaagga

SEQ ID NO: 142 - DNA, 89 bp, Artificial Sequence
  Synthetic
ccttcgatagctcagctggtagagcggaggactctaggcgcgcgcccgtggccatccttaggtcgctggttcgattccggctcgaagga

SEQ ID NO: 143 - DNA, 73 bp, Artificial Sequence; identical to SEQ ID NO: 141
  Synthetic

SEQ ID NO: 144 - DNA, 94 bp, Artificial Sequence
  Synthetic
ccttcgatagctcagctggtagagcggaggactctagcctgtagaaacatttgtggacatccttaggtcgctggttcgattccggctcgaagga

SEQ ID NO: 145 - DNA, 73 bp, Artificial Sequence; identical to SEQ ID NO: 141
  Synthetic

SEQ ID NO: 146 - DNA, 94 bp, Artificial Sequence
  Synthetic
ccttcgatagctcagctggtagagcggaggactctagattgtacagacatttgcggacatccttaggtcgctggttcgattccggctcgaagga

SEQ ID NO: 147 - DNA, 73 bp, Artificial Sequence; identical to SEQ ID NO: 141
  Synthetic

SEQ ID NO: 148 - DNA, 89 bp, Artificial Sequence
  Synthetic
ccttcgatagctcagctggtagagcggaggactctagtacttaatgtgtggtcatccttaggtcgctggttcgattccggctcgaagga

SEQ ID NO: 149 - DNA, 73 bp, Artificial Sequence; identical to SEQ ID NO: 141
  Synthetic

SEQ ID NO: 150 - DNA, 89 bp, Artificial Sequence
  Synthetic
ccttcgatagctcagctggtagagcggaggactctaggggtttgaatgtggtcatccttaggtcgctggttcgaatccggctcggagga

SEQ ID NO: 151 - DNA, 73 bp, Artificial Sequence
  Synthetic
ccttcgatagctcagctggtagagcggaggactctagatccttaggtcgctggttcgaatccggctcggagga

SEQ ID NO: 152 - DNA, 94 bp, Artificial Sequence
  Synthetic
ccttcgatagctcagctggtagagcggaggactctagactgcggaaacgtttgtggacatccttaggtcgctggttcaattccggctcgaagga

SEQ ID NO: 153 - DNA, 73 bp, Artificial Sequence
  Synthetic
ccttcgatagctcagctggtagagcggaggactctagatccttaggtcgctggttcaattccggctcgaagga

SEQ ID NO: 154 - DNA, 90 bp, Artificial Sequence
  Synthetic
ctttcgatagctcagttggtagagcggaggactctaggttcattaaactaaggcatccttaggtcgctggttcgaatccggctcgaagga

SEQ ID NO: 155 - DNA, 73 bp, Artificial Sequence
  Synthetic
ctttcgatagctcagttggtagagcggaggactctagatccttaggtcgctggttcgaatccggctcgaagga

SEQ ID NO: 156 - DNA, 88 bp, Artificial Sequence
  Synthetic
tcttcaatagctcagctggtagagcggaggactctaggtgcacgcccgtggccattcttaggtgctggtttgattccgacttggagag

SEQ ID NO: 157 - DNA, 82 bp, Artificial Sequence
  Synthetic
ggtagcgtggccgagcggtctaaggcgctggattttagctccagtctcttcgggggcgtgggttcaaatcccaccgctgcca

SEQ ID NO: 158 - DNA, 83 bp, Artificial Sequence
  Synthetic
gtcaggatggccgagtggtctaaggcgccagactttagttctggtctccaatggaggcgtgggttcgaatcccacttctgaca

SEQ ID NO: 159 - DNA, 105 bp, Artificial Sequence
  Synthetic
gtcaggatggccgagtggtctaaggcgccagactttagcttggcttcctcgtgttgaggattctggtctccaatggaggcgtgggttcgaatcccacttctgaca

SEQ ID NO: 160 - DNA, 83 bp, Artificial Sequence; identical to SEQ ID NO: 158
  Synthetic

SEQ ID NO: 161 - DNA, 108 bp, Artificial Sequence
  Synthetic
gtcaggatggccgagtggtctaaggcgccagactttagcttactgcttcctgtgttcgggtcttctggtctccgtatggaggcgtgggttcgaatcccacttctgaca

SEQ ID NO: 162 - DNA, 84 bp, Artificial Sequence
  Synthetic
gtcaggatggccgagtggtctaaggcgccagactttagttctggtctccgtatggaggcgtgggttcgaatcccacttctgaca

SEQ ID NO: 163 - DNA, 107 bp, Artificial Sequence
  Synthetic
gtcaggatggccgagtggtctaaggcgccagactttagttgctacttcccaggtttggggcttctggtctccgcatggaggcgtgggttcgaatcccacttctgaca

SEQ ID NO: 164 - DNA, 84 bp, Artificial Sequence
  Synthetic
gtcaggatggccgagtggtctaaggcgccagactttagttctggtctccgcatggaggcgtgggttcgaatcccacttctgaca

SEQ ID NO: 165 - DNA, 106 bp, Artificial Sequence
  Synthetic
gtcaggatggccgagtggtctaaggcgccagactttaggtaagcaccttgcctgcgggctttctggtctccggatggaggcgtgggttcgaatcccacttctgaca

SEQ ID NO: 166 - DNA, 74 bp, Artificial Sequence
  Synthetic
gcctccttagtgcagtaggtagcgcatcagtctttaaatctgaatggtcctgagttcaagcctcagagggggca

SEQ ID NO: 167 - DNA, 83 bp, Artificial Sequence
  Synthetic
accaggatggccgagtggttaaggcgttggactttagatccaatggacatatgtccgcgtgggttcgaaccccactcctggta

SEQ ID NO: 168 - DNA, 83 bp, Artificial Sequence
  Synthetic
accgggatggccgagtggttaaggcgttggactttagatccaatgggctggtgcccgcgtgggttcgaaccccactctcggta

SEQ ID NO: 169 - DNA, 83 bp, Artificial Sequence
  Synthetic
accagaatggccgagtggttaaggcgttggactttagatccaatggattcatatccgcgtgggttcgaaccccacttctggta

SEQ ID NO: 170 - DNA, 83 bp, Artificial Sequence
  Synthetic
accgggatggctgagtggttaaggcgttggactttagatccaatggacaggtgtccgcgtgggttcgagccccactcccggta

SEQ ID NO: 171 - DNA, 83 bp, Artificial Sequence
  Synthetic
actcatttggctgagtggttaaggcattggactttagatccaatggagtagtggctgtgtgggtttaaaccccactactggta

SEQ ID NO: 172 - DNA, 69 bp, Artificial Sequence
  Synthetic
gagaaagtcatcgtagttacgaagttggctttaacccagttttgggaggttcaattccttcctttctct

SEQ ID NO: 173 - DNA, 84 bp, Artificial Sequence
  Synthetic
accaggatggccaagtagttaaaggcactggactttagagccaatggacatatgtctgtgtgggtttgaaccccactcctggtg

SEQ ID NO: 174 - DNA, 82 bp, Artificial Sequence
  Synthetic
ggtagcgtggccgagcggtctaaggcgctggattttagctccagtctcttcggaggcgtgggttcgaatcccaccgctgcca

SEQ ID NO: 175 - DNA, 82 bp, Artificial Sequence
  Synthetic
ggtagtgtggccgagcggtctaaggcgctggattttagctccagtctcttcgggggcgtgggttcgaatcccaccactgcca

SEQ ID NO: 176 - DNA, 82 bp, Artificial Sequence
  Synthetic
ggtagcgtggccgagtggtctaaggcgctggattttagctccagtcatttcgatggcgtgggttcgaatcccaccgctgcca

SEQ ID NO: 177 - DNA, 82 bp, Artificial Sequence
  Synthetic
ggtagtgtggttgaatggtctaaggcactgaattttagctccagtctctttggggacgtgggtttaaatcccactgctgcaa

SEQ ID NO: 178 - DNA, 75 bp, Artificial Sequence
  Synthetic
gttaagatggcagagcctggtaattgcactaaacttaaaattttataatcagaggttcaactcctcttcttaaca

SEQ ID NO: 179 - DNA, 75 bp, Artificial Sequence
  Synthetic
gttaagatggcagagcccggcaattgcactagacttaaaactttataatcagaggttcaactcctctcattaaca

SEQ ID NO: 180 - DNA, 82 bp, Artificial Sequence
  Synthetic
ggtagcgtggccgagcggtctaaggcgctggattctagctccagtctcttcgggggcgtgggttcaaatcccaccgctgcca

SEQ ID NO: 181 - DNA, 82 bp, Artificial Sequence
  Synthetic
ggtagcgtggccgagtggtctaagacgctggattctagctccagtctcttcgggggcgtgggtttgaatcccaccgctgcca

SEQ ID NO: 182 - DNA, 65 bp, Artificial Sequence
  Synthetic
gccgagcggtctaaggctccggattctagcgccggtgtcttcggaggcatgggttcgaattccac

SEQ ID NO: 183 - DNA, 106 bp, Artificial Sequence
  Synthetic
gtcaggatggccgagtggtctaaggcgccagactctagctaagcttcctccgcggtggggattctggtctccaatggaggcgtgggttcgaatcccacttctgaca

SEQ ID NO: 184 - DNA, 83 bp, Artificial Sequence
  Synthetic
gtcaggatggccgagtggtctaaggcgccagactctagttctggtctccaatggaggcgtgggttcgaatcccacttctgaca

SEQ ID NO: 185 - DNA, 105 bp, Artificial Sequence
  Synthetic
gtcaggatggccgagtggtctaaggcgccagactctagcttggcttcctcgtgttgaggattctggtctccaatggaggcgtgggttcgaatcccacttctgaca

SEQ ID NO: 186 - DNA, 83 bp, Artificial Sequence; identical to SEQ ID NO: 184
  Synthetic

SEQ ID NO: 187 - DNA, 108 bp, Artificial Sequence
  Synthetic
gtcaggatggccgagtggtctaaggcgccagactctagcttactgcttcctgtgttcgggtcttctggtctccgtatggaggcgtgggttcgaatcccacttctgaca

SEQ ID NO: 188 - DNA, 84 bp, Artificial Sequence
  Synthetic
gtcaggatggccgagtggtctaaggcgccagactctagttctggtctccgtatggaggcgtgggttcgaatcccacttctgaca

SEQ ID NO: 189 - DNA, 107 bp, Artificial Sequence
  Synthetic
gtcaggatggccgagtggtctaaggcgccagactctagttgctacttcccaggtttggggcttctggtctccgcatggaggcgtgggttcgaatcccacttctgaca

SEQ ID NO: 190 - DNA, 84 bp, Artificial Sequence
  Synthetic
gtcaggatggccgagtggtctaaggcgccagactctagttctggtctccgcatggaggcgtgggttcgaatcccacttctgaca

SEQ ID NO: 191 - DNA, 106 bp, Artificial Sequence
  Synthetic
gtcaggatggccgagtggtctaaggcgccagactctaggtaagcaccttgcctgcgggctttctggtctccggatggaggcgtgggttcgaatcccacttctgaca

SEQ ID NO: 192 - DNA, 74 bp, Artificial Sequence
  Synthetic
gcctccttagtgcagtaggtagcgcatcagtctctaaatctgaatggtcctgagttcaagcctcagagggggca

SEQ ID NO: 193 - DNA, 83 bp, Artificial Sequence
  Synthetic
accaggatggccgagtggttaaggcgttggactctagatccaatggacatatgtccgcgtgggttcgaaccccactcctggta

SEQ ID NO: 194 - DNA, 83 bp, Artificial Sequence
  Synthetic
accgggatggccgagtggttaaggcgttggactctagatccaatgggctggtgcccgcgtgggttcgaaccccactctcggta

SEQ ID NO: 195 - DNA, 83 bp, Artificial Sequence
  Synthetic
accagaatggccgagtggttaaggcgttggactctagatccaatggattcatatccgcgtgggttcgaaccccacttctggta

SEQ ID NO: 196 - DNA, 83 bp, Artificial Sequence
  Synthetic
accgggatggctgagtggttaaggcgttggactctagatccaatggacaggtgtccgcgtgggttcgagccccactcccggta

SEQ ID NO: 197 - DNA, 83 bp, Artificial Sequence
  Synthetic
actcatttggctgagtggttaaggcattggactctagatccaatggagtagtggctgtgtgggtttaaaccccactactggta

SEQ ID NO: 198 - DNA, 82 bp, Artificial Sequence
  Synthetic
ggtagcgtggccgagcggtctaaggcgctggattctagctccagtctcttcggaggcgtgggttcgaatcccaccgctgcca

SEQ ID NO: 199 - DNA, 82 bp, Artificial Sequence
  Synthetic
ggtagtgtggccgagcggtctaaggcgctggattctagctccagtctcttcgggggcgtgggttcgaatcccaccactgcca

SEQ ID NO: 200 - DNA, 82 bp, Artificial Sequence
  Synthetic
ggtagcgtggccgagtggtctaaggcgctggattctagctccagtcatttcgatggcgtgggttcgaatcccaccgctgcca

SEQ ID NO: 201 - DNA, 82 bp, Artificial Sequence
  Synthetic
ggtagtgtggttgaatggtctaaggcactgaattctagctccagtctctttggggacgtgggtttaaatcccactgctgcaa

SEQ ID NO: 202 - DNA, 82 bp, Artificial Sequence
  Synthetic
ggtagcgtggccgagcggtctaaggcgctggatttcagctccagtctcttcgggggcgtgggttcaaatcccaccgctgcca

SEQ ID NO: 203 - DNA, 106 bp, Artificial Sequence
  Synthetic
gtcaggatggccgagtggtctaaggcgccagacttcagctaagcttcctccgcggtggggattctggtctccaatggaggcgtgggttcgaatcccacttctgaca

SEQ ID NO: 204 - DNA, 83 bp, Artificial Sequence
  Synthetic
gtcaggatggccgagtggtctaaggcgccagacttcagttctggtctccaatggaggcgtgggttcgaatcccacttctgaca

SEQ ID NO: 205 - DNA, 105 bp, Artificial Sequence
  Synthetic
gtcaggatggccgagtggtctaaggcgccagacttcagcttggcttcctcgtgttgaggattctggtctccaatggaggcgtgggttcgaatcccacttctgaca

SEQ ID NO: 206 - DNA, 83 bp, Artificial Sequence; identical to SEQ ID NO: 204
  Synthetic

SEQ ID NO: 207 - DNA, 108 bp, Artificial Sequence
  Synthetic
gtcaggatggccgagtggtctaaggcgccagacttcagcttactgcttcctgtgttcgggtcttctggtctccgtatggaggcgtgggttcgaatcccacttctgaca

SEQ ID NO: 208 - DNA, 84 bp, Artificial Sequence
  Synthetic
gtcaggatggccgagtggtctaaggcgccagacttcagttctggtctccgtatggaggcgtgggttcgaatcccacttctgaca

SEQ ID NO: 209 - DNA, 107 bp, Artificial Sequence
  Synthetic
gtcaggatggccgagtggtctaaggcgccagacttcagttgctacttcccaggtttggggcttctggtctccgcatggaggcgtgggttcgaatcccacttctgaca

SEQ ID NO: 210 - DNA, 84 bp, Artificial Sequence
  Synthetic
gtcaggatggccgagtggtctaaggcgccagacttcagttctggtctccgcatggaggcgtgggttcgaatcccacttctgaca

SEQ ID NO: 211 - DNA, 106 bp, Artificial Sequence
  Synthetic
gtcaggatggccgagtggtctaaggcgccagacttcaggtaagcaccttgcctgcgggctttctggtctccggatggaggcgtgggttcgaatcccacttctgaca

SEQ ID NO: 212 - DNA, 83 bp, Artificial Sequence
  Synthetic
accaggatggccgagtggttaaggcgttggacttcagatccaatggacatatgtccgcgtgggttcgaaccccactcctggta

SEQ ID NO: 213 - DNA, 83 bp, Artificial Sequence
  Synthetic
accgggatggccgagtggttaaggcgttggacttcagatccaatgggctggtgcccgcgtgggttcgaaccccactctcggta

SEQ ID NO: 214 - DNA, 83 bp, Artificial Sequence
  Synthetic
accagaatggccgagtggttaaggcgttggacttcagatccaatggattcatatccgcgtgggttcgaaccccacttctggta

SEQ ID NO: 215 - DNA, 83 bp, Artificial Sequence
  Synthetic
accgggatggctgagtggttaaggcgttggacttcagatccaatggacaggtgtccgcgtgggttcgagccccactcccggta

SEQ ID NO: 216 - DNA, 83 bp, Artificial Sequence
  Synthetic
actcatttggctgagtggttaaggcattggacttcagatccaatggagtagtggctgtgtgggtttaaaccccactactggta

SEQ ID NO: 217 - DNA, 82 bp, Artificial Sequence
  Synthetic
ggtagcgtggccgagcggtctaaggcgctggatttcagctccagtctcttcggaggcgtgggttcgaatcccaccgctgcca

SEQ ID NO: 218 - DNA, 82 bp, Artificial Sequence
  Synthetic
ggtagtgtggccgagcggtctaaggcgctggatttcagctccagtctcttcgggggcgtgggttcgaatcccaccactgcca

SEQ ID NO: 219 - DNA, 82 bp, Artificial Sequence
  Synthetic
ggtagcgtggccgagtggtctaaggcgctggatttcagctccagtcatttcgatggcgtgggttcgaatcccaccgctgcca

SEQ ID NO: 220 - DNA, 70 bp, Artificial Sequence
  Synthetic
gagaaggtcatagaggttatgggattggctctaaaccagtctctggggggttcgattccctcctttttca

SEQ ID NO: 221 - DNA, 82 bp, Artificial Sequence
  Synthetic
gtagtcgtggccgagtggttaaggcgatggactctaaatccattggggtttccccgcgcaggttcgaatcctgccgactacg

SEQ ID NO: 222 - DNA, 82 bp, Artificial Sequence
  Synthetic
gtagtcgtggccgagtggttaaggcgatggactctaaatccattggggtttccccacgcaggttcgaatcctgccgactacg

SEQ ID NO: 223 - DNA, 82 bp, Artificial Sequence
  Synthetic
gtagtcgtggccgagtggttaaggtgatggactctaaacccattggggtctccccgcgcaggttcgaatcctgccgactacg

SEQ ID NO: 224 - DNA, 72 bp, Artificial Sequence
  Synthetic
gggtgtatggctcaggggtagagaatttgactctagatcaagaggtccctggttcaaatccaggtgccccct

SEQ ID NO: 225 - DNA, 78 bp, Artificial Sequence
  Synthetic
agttgtagctgagtggttaaggcaacgagctctaaattcgttggtttctctctgtgcaggtttgaatcctgctaatta

SEQ ID NO: 226 - DNA, 70 bp, Artificial Sequence
  Synthetic
caagaaattcatagaggttatgggattggctctaaaccagtttcaggaggttcgattccttcctttttgg

SEQ ID NO: 227 - DNA, 82 bp, Artificial Sequence
  Synthetic
gctgtgatggccgagtggttaaggcgttggactctaaatccaatggggtctccccgcgcaggttcaaatcctgctcacagcg

SEQ ID NO: 228 - DNA, 82 bp, Artificial Sequence
  Synthetic
gctgtgatggccgagtggttaaggtgttggactctaaatccaatgggggttccccgcgcaggttcaaatcctgctcacagcg

SEQ ID NO: 229 - DNA, 82 bp, Artificial Sequence
  Synthetic
gtcacggtggccgagtggttaaggcgttggactctaaatccaatggggtttccccgcacaggttcgaatcctgttcgtgacg

SEQ ID NO: 230 - DNA, 82 bp, Artificial Sequence
  Synthetic
gacgaggtggccgagtggttaaggcgatggactctaaatccattgtgctctgcacgcgtgggttcgaatcccaccctcgtcg

SEQ ID NO: 231 - DNA, 82 bp, Artificial Sequence
  Synthetic
gacgaggtggccgagtggttaaggcgatggactctaaatccattgtgctctgcacgcgtgggttcgaatcccaccttcgtcg

SEQ ID NO: 232 - DNA, 74 bp, Artificial Sequence
  Synthetic
ggccggttagctcagttggttagagcgtgctctaactaatgccagggtcgaggtttcgatccccgtacgggcct

SEQ ID NO: 233 - DNA, 82 bp, Artificial Sequence
  Synthetic
gacgaggtggccgagtggttaaggcgatggactctaaatccattgtgctctgcacacgtgggttcgaatcccatcctcgtcg

SEQ ID NO: 234 - DNA, 78 bp, Artificial Sequence
  Synthetic
gaggcctggccgagtggttaaggcgatggactctaaatccattgtgctctgcacgcgtgggttcgaatcccatcctcg

SEQ ID NO: 235 - DNA, 82 bp, Artificial Sequence
  Synthetic
gcagcgatggccgagtggttaaggcgttggactctaaatccaatggggtctccccgcgcaggttcgaaccctgctcgctgcg

SEQ ID NO: 236 - DNA, 82 bp, Artificial Sequence; identical to SEQ ID NO: 221
  Synthetic

SEQ ID NO: 237 - DNA, 82 bp, Artificial Sequence
  Synthetic
gtagtcgtggccgagtggttaaggcgatggactctaaatccattggggtctccccgcgcaggttcgaatcctgccgactacg

SEQ ID NO: 238 - DNA, 82 bp, Artificial Sequence
  Synthetic
gtagtcgtggccgagtggttaaggcgatggactctaaatccattggggtttccccgcgcaggttcgaatcctgtcggctacg

SEQ ID NO: 239 - DNA, 82 bp, Artificial Sequence
  Synthetic
gtagtcgtggccgagtggttaaggcgatggacttcaaatccattggggtttccccgcgcaggttcgaatcctgccgactacg

SEQ ID NO: 240 - DNA, 82 bp, Artificial Sequence
  Synthetic
gtagtcgtggccgagtggttaaggcgatggacttcaaatccattggggtttccccacgcaggttcgaatcctgccgactacg

SEQ ID NO: 241 - DNA, 82 bp, Artificial Sequence
  Synthetic
gtagtcgtggccgagtggttaaggtgatggacttcaaacccattggggtctccccgcgcaggttcgaatcctgccgactacg

SEQ ID NO: 242 - DNA, 78 bp, Artificial Sequence
  Synthetic
agttgtagctgagtggttaaggcaacgagcttcaaattcgttggtttctctctgtgcaggtttgaatcctgctaatta

SEQ ID NO: 243 - DNA, 82 bp, Artificial Sequence
  Synthetic
gctgtgatggccgagtggttaaggcgttggacttcaaatccaatggggtctccccgcgcaggttcgaatcctgctcacagcg

SEQ ID NO: 244 - DNA, 82 bp, Artificial Sequence
  Synthetic
gctgtgatggccgagtggttaaggcgttggacttcaaatccaatggggtctccccgcgcaggttcaaatcctgctcacagcg

SEQ ID NO: 245 - DNA, 82 bp, Artificial Sequence
  Synthetic
gctgtgatggccgagtggttaaggtgttggacttcaaatccaatgggggttccccgcgcaggttcaaatcctgctcacagcg

SEQ ID NO: 246 - DNA, 82 bp, Artificial Sequence
  Synthetic
gtcacggtggccgagtggttaaggcgttggacttcaaatccaatggggtttccccgcacaggttcgaatcctgttcgtgacg

SEQ ID NO: 247 - DNA, 82 bp, Artificial Sequence
  Synthetic
gacgaggtggccgagtggttaaggcgatggacttcaaatccattgtgctctgcacgcgtgggttcgaatcccaccctcgtcg

SEQ ID NO: 248 - DNA, 82 bp, Artificial Sequence
  Synthetic
gacgaggtggccgagtggttaaggcgatggacttcaaatccattgtgctctgcacgcgtgggttcgaatcccaccttcgtcg

SEQ ID NO: 249 - DNA, 74 bp, Artificial Sequence
  Synthetic
ggccggttagctcagttggttagagcgtgcttcaactaatgccagggtcgaggtttcgatccccgtacgggcct

SEQ ID NO: 250 - DNA, 82 bp, Artificial Sequence
  Synthetic
gacgaggtggccgagtggttaaggcgatggacttcaaatccattgtgctctgcacacgtgggttcgaatcccatcctcgtcg

SEQ ID NO: 251 - DNA, 82 bp, Artificial Sequence
  Synthetic
gcagcgatggccgagtggttaaggcgttggacttcaaatccaatggggtctccccgcgcaggttcgaaccctgctcgctgcg

SEQ ID NO: 252 - DNA, 82 bp, Artificial Sequence; identical to SEQ ID NO: 239
  Synthetic

SEQ ID NO: 253 - DNA, 82 bp, Artificial Sequence
  Synthetic
gtagtcgtggccgagtggttaaggcgatggacttcaaatccattggggtctccccgcgcaggttcgaatcctgccgactacg

SEQ ID NO: 254 - DNA, 82 bp, Artificial Sequence
  Synthetic
gtagtcgtggccgagtggttaaggcgatggacttcaaatccattggggtttccccgcgcaggttcgaatcctgtcggctacg

SEQ ID NO: 255 - DNA, 82 bp, Artificial Sequence
  Synthetic
gtagtcgtggccgagtggttaaggcgatggactttaaatccattggggtttccccgcgcaggttcgaatcctgccgactacg

SEQ ID NO: 256 - DNA, 82 bp, Artificial Sequence
  Synthetic
gtagtcgtggccgagtggttaaggcgatggactttaaatccattggggtttccccacgcaggttcgaatcctgccgactacg

SEQ ID NO: 257 - DNA, 82 bp, Artificial Sequence
  Synthetic
gtagtcgtggccgagtggttaaggtgatggactttaaacccattggggtctccccgcgcaggttcgaatcctgccgactacg

SEQ ID NO: 258 - DNA, 82 bp, Artificial Sequence
  Synthetic
gctgtgatggccgagtggttaaggcgttggactttaaatccaatggggtctccccgcgcaggttcgaatcctgctcacagcg

SEQ ID NO: 259 - DNA, 82 bp, Artificial Sequence
  Synthetic
gctgtgatggccgagtggttaaggcgttggactttaaatccaatggggtctccccgcgcaggttcaaatcctgctcacagcg

SEQ ID NO: 260 - DNA, 82 bp, Artificial Sequence
  Synthetic
gctgtgatggccgagtggttaaggtgttggactttaaatccaatgggggttccccgcgcaggttcaaatcctgctcacagcg

SEQ ID NO: 261 - DNA, 82 bp, Artificial Sequence
  Synthetic
gtcacggtggccgagtggttaaggcgttggactttaaatccaatggggtttccccgcacaggttcgaatcctgttcgtgacg

SEQ ID NO: 262 - DNA, 82 bp, Artificial Sequence
  Synthetic
gacgaggtggccgagtggttaaggcgatggactttaaatccattgtgctctgcacgcgtgggttcgaatcccaccctcgtcg

SEQ ID NO: 263 - DNA, 82 bp, Artificial Sequence
  Synthetic
gacgaggtggccgagtggttaaggcgatggactttaaatccattgtgctctgcacgcgtgggttcgaatcccaccttcgtcg

SEQ ID NO: 264 - DNA, 74 bp, Artificial Sequence
  Synthetic
ggccggttagctcagttggttagagcgtgctttaactaatgccagggtcgaggtttcgatccccgtacgggcct

SEQ ID NO: 265 - DNA, 82 bp, Artificial Sequence
  Synthetic
gacgaggtggccgagtggttaaggcgatggactttaaatccattgtgctctgcacacgtgggttcgaatcccatcctcgtcg

SEQ ID NO: 266 - DNA, 78 bp, Artificial Sequence
  Synthetic
gaggcctggccgagtggttaaggcgatggactttaaatccattgtgctctgcacgcgtgggttcgaatcccatcctcg

SEQ ID NO: 267 - DNA, 82 bp, Artificial Sequence
  Synthetic
gcagcgatggccgagtggttaaggcgttggactttaaatccaatggggtctccccgcgcaggttcgaaccctgctcgctgcg

SEQ ID NO: 268 - DNA, 82 bp, Artificial Sequence; identical to SEQ ID NO: 255
  Synthetic

SEQ ID NO: 269 - DNA, 82 bp, Artificial Sequence
  Synthetic
gtagtcgtggccgagtggttaaggcgatggactttaaatccattggggtctccccgcgcaggttcgaatcctgccgactacg

SEQ ID NO: 270 - DNA, 82 bp, Artificial Sequence
  Synthetic
gtagtcgtggccgagtggttaaggcgatggactttaaatccattggggtttccccgcgcaggttcgaatcctgtcggctacg

SEQ ID NO: 271 - DNA, 73 bp, Artificial Sequence
  Synthetic
gcccagctagctcagtcggtagagcataagactttaaatctcagggttgtggattcgtgccccatgctgggtg

SEQ ID NO: 272 - DNA, 73 bp, Artificial Sequence
  Synthetic
gcccggctagctcagtcggtagagcatgagaccttaaatctcagggtcgtgggttcgagccccacgttgggcg

SEQ ID NO: 273 - DNA, 73 bp, Artificial Sequence
  Synthetic
gcccggctagctcagtcggtagagcatgggactttaaatctcagggtcgtgggttcgagccccacgttgggcg

SEQ ID NO: 274 - DNA, 73 bp, Artificial Sequence
  Synthetic
gcccggctagctcagtcgatagagcatgagactttaaatctcagggtcgtgggttcgagccgcacgttgggcg

SEQ ID NO: 275 - DNA, 73 bp, Artificial Sequence
  Synthetic
gcctggctagctcagtcggcaaagcatgagactttaaatctcagggtcgtgggctcgagctccatgttgggcg

SEQ ID NO: 276 - DNA, 73 bp, Artificial Sequence
  Synthetic
gcctggatagctcagttggtagagcatcagactttaaatctgagggtccagggttcaagtccctgttcaggca

SEQ ID NO: 277 - DNA, 73 bp, Artificial Sequence
  Synthetic
acctgggtagcttagttggtagagcattggactttaaatttgagggcccaggtttcaagtccctgtttgggtg

SEQ ID NO: 278 - DNA, 73 bp, Artificial Sequence
  Synthetic
gttggggtaactcagttggtagagtagcagactttacatctgagggtccagggtttaagtccatgtccaggca

SEQ ID NO: 279 - DNA, 73 bp, Artificial Sequence
  Synthetic
gcctggatagctcagttggtagagcatcagactttaaatctgagggtccagggttcaagtccctgttcaggcg

SEQ ID NO: 280 - DNA, 73 bp, Artificial Sequence
  Synthetic
gcctggatagctcagtcggtagagcatcagactttaaatctgagggtccagggttcaagtccctgttcaggcg

SEQ ID NO: 281 - DNA, 73 bp, Artificial Sequence
  Synthetic
gcccggatagctcagtcggtagagcatcagactttaaatctgagggtccggggttcaagtccctgttcgggcg

SEQ ID NO: 282 - DNA, 73 bp, Artificial Sequence
  Synthetic
gcctgggtagctcagtcggtagagcatcagactttaaatctgagggtccagggttcaagtccctgtccaggcg

SEQ ID NO: 283 - DNA, 73 bp, Artificial Sequence
  Synthetic
gcctggatagctcagttggtagaacatcagactttaaatctgacggtgcagggttcaagtccctgttcaggcg

SEQ ID NO: 284 - DNA, 73 bp, Artificial Sequence
  Synthetic
acctgggtagctcagtaggtagaacatcagactttaaatctgagggtctagggttcaagtccctgtccaggcg

SEQ ID NO: 285 - DNA, 73 bp, Artificial Sequence
  Synthetic
gcccagctagctcagtcggtagagcataagactctaaatctcagggttgtggattcgtgccccatgctgggtg

SEQ ID NO: 286 - DNA, 73 bp, Artificial Sequence
  Synthetic
gcccggctagctcagtcggtagagcatgagaccctaaatctcagggtcgtgggttcgagccccacgttgggcg

SEQ ID NO: 287 - DNA, 73 bp, Artificial Sequence
  Synthetic
gcccggctagctcagtcggtagagcatgggactctaaatctcagggtcgtgggttcgagccccacgttgggcg

SEQ ID NO: 288 - DNA, 73 bp, Artificial Sequence
  Synthetic
gcctggatagctcagttggtagagcatcagactctaaatctgagggtccagggttcaagtccctgttcaggca

SEQ ID NO: 289 - DNA, 73 bp, Artificial Sequence
  Synthetic
acctgggtagcttagttggtagagcattggactctaaatttgagggcccaggtttcaagtccctgtttgggtg

SEQ ID NO: 290 - DNA, 73 bp, Artificial Sequence
  Synthetic
gcctggatagctcagttggtagagcatcagactctaaatctgagggtccagggttcaagtccctgttcaggcg

SEQ ID NO: 291 - DNA, 73 bp, Artificial Sequence
  Synthetic
gcctggatagctcagtcggtagagcatcagactctaaatctgagggtccagggttcaagtccctgttcaggcg

SEQ ID NO: 292 - DNA, 73 bp, Artificial Sequence
  Synthetic
gcccggatagctcagtcggtagagcatcagactctaaatctgagggtccggggttcaagtccctgttcgggcg

SEQ ID NO: 293 - DNA, 73 bp, Artificial Sequence
  Synthetic
gcctgggtagctcagtcggtagagcatcagactctaaatctgagggtccagggttcaagtccctgtccaggcg

SEQ ID NO: 294 - DNA, 73 bp, Artificial Sequence
  Synthetic
gcctggatagctcagttggtagaacatcagactctaaatctgacggtgcagggttcaagtccctgttcaggcg

SEQ ID NO: 295 - DNA, 72 bp, Artificial Sequence
  Synthetic
gggggcatagctcagtggtagagcatttgacttcagatcaagaggtccctggttcaaatccaggtgccccct

SEQ ID NO: 296 - DNA, 72 bp, Artificial Sequence
  Synthetic
gggggtatagcttagcggtagagcatttgacttcagatcaagaggtccccggttcaaatccgggtgccccct

SEQ ID NO: 297 - DNA, 72 bp, Artificial Sequence
  Synthetic
gggggtatagcttaggggtagagcatttgacttcagatcaaaaggtccctggttcaaatccaggtgcccctt

SEQ ID NO: 298 - DNA, 72 bp, Artificial Sequence
  Synthetic
gggggtatagctcaggggtagagcatttgacttcagatcaagaggtccccagttcaaatctgggtgccccct

SEQ ID NO: 299 - DNA, 72 bp, Artificial Sequence
  Synthetic
gggggtatagctcaggggtagagcatttgacttcagatcaagaggtctctggttcaaatccaggtgccccct

SEQ ID NO: 300 - DNA, 72 bp, Artificial Sequence
  Synthetic
gggggtatagctcaggggtagagcacttgacttcagatcaagaggtccctggttcaaatccaggtgccccct

SEQ ID NO: 301 - DNA, 72 bp, Artificial Sequence
  Synthetic
gggggtatagctcagtggtagagcatttgacttcagatcaagaggtccctggttcaaatccgggtgccccct

SEQ ID NO: 302 - DNA, 73 bp, Artificial Sequence
  Synthetic
gggggtatagctcagtgggtagagcatttgacttcagatcaagaggtccccggttcaaatccgggtgccccct

SEQ ID NO: 303 - DNA, 72 bp, Artificial Sequence
  Synthetic
gggggtgtagctcagtggtagagcatttgacttcagatcaagaggtccctggttcaaatccaggtgccccct

SEQ ID NO: 304 - DNA, 72 bp, Artificial Sequence
  Synthetic
gggggtatagctcaggggtagagcatttgacttcagatcaagaggtccccggttcaaatccgggtgccccct

SEQ ID NO: 305 - DNA, 72 bp, Artificial Sequence
  Synthetic
gggggtatagctcaggggtagagcatttgacttcagatcaagaggtccctggttcaaatccaggtgccccct

SEQ ID NO: 306 - DNA, 55 bp, Artificial Sequence
  Synthetic
agcgctccggtttttctgtgctgaacctcaggggacgccgacacacgtacacgtc

SEQ ID NO: 307 - DNA, 87 bp, Artificial Sequence
  Synthetic
atgctttgcatacttctgcctgctggggagcctggggactttccacaccctaactgacacacattccacagctggttggtacctgca

(19) United States
(12) Patent Application Publication
(10) Pub. No.: US 2023/0272378 A1
(43) Pub. Date: Aug. 31, 2023

(54) ENCODING AND EXPRESSION OF ACE-tRNAs

(71) Applicant: University of Rochester, Rochester, NY (US)

(72) Inventors: John D. Lueck, Rochester, NY (US); Joseph J. Porter, Rochester, NY (US); Charles A. Thornton, Rochester, NY (US); Wooree Ko, Rochester, NY (US)

(73) Assignee: University of Rochester, Rochester, NY (US)

(21) Appl. No.: 18/000,706

(22) PCT Filed: Jun. 7, 2021
(86) PCT No.: PCT/US2021/036165

§ 371 Date: Dec. 5, 2022

Related U.S. Application Data
(60) Provisional application No. 63/038,245, filed on Jun. 12, 2020.

(51) Int. Cl. C12N 15/11

(52) U.S. Cl. CPC C12N 15/11; C12N 2310/532

(57) ABSTRACT

This invention relates to compositions and methods for treating a disease or disorder associated with premature termination codon. Certain aspects of the invention relate to polynucleotides, vectors, and host cells, and uses thereof.

### CROSS REFERENCE TO RELATED APPLICATION

This application is the U.S. national phase of International Patent Application No. PCT/US2021/036165 filed Jun. 7, 2021, which claims priority to U.S. Provisional Application No. 63/038,245 filed Jun. 12, 2020, the disclosures of which are incorporated herein by reference.

### GOVERNMENT INTERESTS

This invention was made with government support under HL153988 awarded by National Institutes of Health. The government has certain rights in the invention.

### REFERENCE TO A SEQUENCE LISTING

This application incorporates by reference the Sequence Listing submitted in Computer Readable Form as file SeqListing_161118-02102_ST25, created on Apr. 17, 2023 and containing 80,479 bytes.

### FIELD OF THE INVENTION

This invention relates to anti-codon edited (ACE)-tRNAs based agents and methods for treating disorders associated with premature termination codon (PTC).

### BACKGROUND OF THE INVENTION

The genetic code uses 4 nucleotides that form triplet “codons” which are the basis for DNA to protein translation. There are 64 codons in total, 61 of which are used to encode amino acids and 3 (TAG, TGA and TAA) encode translation termination signals. Nonsense mutations change an amino acid codon to PTC generally through a single-nucleotide substitution, resulting in a defective truncated protein and severe forms of disease. Nonsense mutations account for greater than 10% of all genetic diseases and nearly 1,000 genetic human disorders, including cancer that affects about 300 million people worldwide. Indeed, cystic fibrosis (CF) follows suit with about 22% of all CF patients having “class 1” PTC mutations (e.g., p.G542X, p.R553X and p.W1282X), resulting in nearly complete loss of Cystic Fibrosis Transmembrane conductance Regulator (CFTR) function and severe clinical manifestations. Because of the exceedingly high prevalence of nonsense-associated disease, and a unifying mechanism, there has been concerted effort to identify PTC therapeutics. Aminoglycosides (AMGs) have been the major focus of these efforts. However ototoxicity and nephrotoxicity with extended use has restricted their use clinically. Synthetic AMG derivatives are currently being investigated to reduce off-target effects; yet, they often suffer low readthrough efficiency. Non-AMG small-molecules (e.g., tylosin, Ataluren) have also been identified as promising PTC readthrough compounds with little toxicity. However, these approaches have a number of challenges yet to be overcome, including insertion of near-cognate tRNAs that often lead to the generation of a missense mutation at the site of the original PTC. Furthermore, some of the compounds have low efficiency of PTC suppression in human primary cells, which resulted in Ataluren failing phase 3 clinical trials (ACT DMD Phase 3 clinical trial, NCT01826487; ACT CF, NCT02139306). There is a need for therapeutic agents and methods for treatment of disorders associated with nonsense mutations.

### SUMMARY OF INVENTION

This invention addresses the need mentioned above in a number of aspects.

In one aspect, the invention provides a closed end, circular, non-viral, and non-plasmid DNA molecule comprising (1) a promoter and (ii) a sequence encoding an anti-codon edited-tRNA (ACE-tRNA). The molecule can be a closed end DNA thread (CEDT) molecule or a minicircle (MC) molecule. The molecule can further comprise one or more elements selected from the group consisting of a DNA nuclear targeting sequence (DTS), a transcription enhancing 5′ leader sequence (TELS), and an ACE-tRNA Barcoding Sequence (ABS). Examples of the 5′ leader sequence include SEQ ID NO: 306. Examples of the DTS include a SV40-DTS, such as SEQ ID NO: 307. In some embodiments, the molecule is free of any bacterial nucleic acid sequence. The molecule can comprise 4 or less CpG dinucleotides. Preferably, the molecule is free of any CpG dinucleotide. The molecule can be about 200 to about 1,000 bp in size, e.g., about 500 bp in size. The ACE-tRNA can be one selected from the group consisting of TrpTGAchr17.trna39, LeuTGAchr6.trna81, LeuTGAchr6.trna135, LeuTGAchr11.trna4, GlyTGAchr19.trna2, GlyTGAchr1.trna107, GlyTGAchr17.trna9, ArgTGAchr9.trna6/nointron, GlnTAGchr1.trna101, and GlnTAGchr6.trna175. Examples include RNAs comprising the sequences of SEQ ID NOs: 1-10. Additional examples include those encoded by SEQ ID NO: 11-305. In one embodiment, the ACE-tRNA comprises a sequence (i) selected from the group consisting of SEQ ID NO: 1, 4, 5, and 8 or (ii) encoded by one selected from the group consisting of SEQ ID NO: 79 and 94.

The molecule can be used in a method for expressing an ACE-tRNA in a cell. The expressed ACE-tRNA has the function of reverting a PTC to an amino acid during the translation of a mRNA. To that end, the method includes (i) contacting a cell of interest with the molecule described above and (ii) maintaining the cell under conditions permitting expression of the ACE-tRNA. The cell can have a mutant nucleic acid comprising one or more PTCs. In that case, the wild type nucleic acid encodes a fully functional polypeptide. Using the method, the expressed ACE-tRNA rescues the one or more PTCs so as to restore expression of the polypeptide or improve the functional activities of the polypeptide in the cell. For instance, the polypeptide can be cystic fibrosis transmembrane conductance regulator (CFTR) and the mutant nucleic acid encodes a truncated CFTR. In one example, the mutant nucleic acid has a Trp-to-Stop PTC. The ACE-tRNA translates the Trp-to-Stop PTC into a Leu. Within scope of this invention is a host cell comprising one or more of the molecules described above.

The molecule described above can be used in a method for treating PTC-associated disorders. Accordingly, the invention also provides a pharmaceutical formulation comprising (i) the molecule and (ii) a pharmaceutically acceptable carrier. Also provided is a method of treating a disease associated with a PTC in a subject in need thereof. The method includes administering to the subject the molecule or the pharmaceutical composition described above. Examples of the disease include cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, and Polycystic kidney disease. In some example, the disease is an eye disease selected from the group consisting of cone dystrophies, Stargardt's disease (STGD1), cone-rod dystrophy, retinitis pigmentosa (RP), increased susceptibility to age-related macular degeneration, Congenital stationary night blindness 2 (CSNB2), Congenital stationary night blindness 1 (CSNB1), Best Disease, VMD, and Leber congenital amaurosis (LCA16).

The treatment method can be carried out using any suitable methods, including nanoparticles, electroporation, polyethylenimine (PEI), receptor-targeted polyplexes, liposomes, or hydrodynamic injection.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A and 1B are diagrams showing two small ACE-tRNA expression cassettes, which are well suited for therapeutic delivery. The entire expression cassette for ACE-tRNAs, including internal promoter A and B box regions (˜76 base pairs or bps), and short 5′ Transcription Enhancing Leader Sequence (TELS, hashed) is about 125 bps or less in total length.

FIG. 1C shows schematics of a minicircle and a CEDT.

FIGS. 2A and 2B are diagrams showing ribosomal profiling of mRNA transcripts following ACE-tRNA expression. (A) Log 2 fold change of ribosome footprint densities in 3′UTRs between ACE tRNA suppressor and control for transcripts with >5 RPKM (Read Per Kilobase of a gene/Million-mapped reads) in coding regions and >0.5 RPKM in 3′UTRs. Each point represents one gene transcript. Error bar: Mean±SD. (B) Normalized average ribosome footprint occupancy surrounding the stop codons of all transcripts where CDS is the gene coding sequence (each transcript equally weighted). (Inset) Magnified view of (B).

FIG. 3 is a set of photographs and diagrams showing ACE-tRNA Mini Circle and Closed End DNA Thread production scheme.

FIGS. 4A and 4B are diagrams showing ACE-tRNA^{Arg} MCs and CEDTs exhibited robust PTC suppression ability. (A) MCs as small as 200 bps expressed ACE-tRNAs (white bars) in HEK293 cells stably expressing a PTC reporter. (B) CEDTs exhibited robust PTC suppression with co-delivery of PTC reporter in 16HBE14o- cells.

FIGS. 5A, 5B, 5C, 5D, 5E, 5F, and 5G are photographs and diagrams showing that delivery of ACE-tRNA CEDTs to CRISPR/Cas9 modified 16HBE14o- cells significantly rescued CFTR function and inhibits nonsense mediated decay (NMD). (A) Representative image of 16HBE14o- cells transfected on Transwells. (B) Representative Cl⁻ short circuit current traced from WT (Black), p.R1162X 16HBE14oe- cells transfected with empty vector (red) and 500 bp ACE-tRNA^{Arg} CEDTs (blue) in Ussing chamber recordings. (C) Average Forskolin and IBMX and (D) Inhb172 responses showed significant rescue of CFTR function by 500 bp ACE-tRNA^{Arg} CEDT (blue). (E) Following meager (30%) transfection efficiencies, plasmids encoding ACE-tRNA^{Gly} (gray lines), ACE-tRNA^{Arg} (green horizontal lines), and ACE-tRNA^{Leu} (orange vert. lines) all significantly inhibited NMD in p.G542X, p.R1162X and pW1282X 16HBE14o- cells, respectively, as measured by qPCR. Importantly, 500 bp CEDT^{Arg} (green diagonal lines), 800 bp MC^{Arg} (green dots) and 800 bp MC^{Leu} (orange dotes) all gave significant and comparable rescue to plasmid-based expression of their respective CFTR PTCs 48 hours after transfection. All constructs were transfected at equal amounts. *p<0.05; **p<0.0001.

FIGS. 6A and 6B are photographs and diagrams showing that electroporation delivery of minivectors into mouse lungs resulted in efficient transduction of airway epithelia and PTC readthrough. (A) Mouse lung airway epithelia was efficiently transduced with a GFP expression vector (inset) by electroporation. (B) Co-delivery of NLuc-UGA PTC reporter plasmid with plasmid ACE-tRNA^{Arg}, 500 bp CEDT^{Arg}, 800 bp MC^{Leu}, and 800 bp MC^{Arg} resulted in robust PTC suppression.

FIGS. 7A and 7B are a photograph and a diagram showing that 5′ flanking sequences modulated ACE-tRNA expression. (A) Suppression of W1282X-CFTR by ACE-tRNA^{Trp}_{UGA} was enhanced (left lane) by human Tyr TELS, as shown by Western blot (WB) of full-length CFTR protein following transfection of cDNAs encoding W1282X-CFTR and ACE-tRNA^{Trp}_{UGA} with (left lane) and without (right lane) TELS in HEK293 cells. (B) Design of TELS screening HTC/HTS plasmid.

FIGS. 8A, 8B, 8C, and 8D are photographs and a diagram showing that ACE-tRNAs plasmids did not actively transport to the nucleus. (A) Injection of ACE-tRNA plasmid cDNA into the cytoplasm of cells resulted in cytoplasmic localization, while (B) nuclear injection resulted in the formation of foci consistent with transcription. (C) The addition of SV40 DNA targeting sequences to the empty plasmid resulted in nuclear localization 4 hours after cytoplasmic injection. (D) Schematic of ACE-tRNA MC and CEDT with SV40 DTS.

FIG. 9 is a diagram showing a PTC reporter plasmid for determining localization, efficiency and persistence of minivector PTC suppression in lung.

FIGS. 10A, 10B, and 10C are diagrams showing ACE-tRNA barcode technology for measuring transcription activity. (A) Schematic of ACE-tRNA barcode scheme (ABS). (B) qPCR measurements of ACE-tRNA^{Arg} and ACE-tRNA^{Tro} barcodes. (C) PTC suppression activity of ACE-tRNA^{Arg}_{UGA} and ACE-tRNA^{Arg}_{UGA}-barcode.

FIGS. 11A, 11B, and 11C are diagrams showing another ACE-tRNA barcode technology for measuring transcription activity. (A) Schematic of tis ACE-tRNA barcode scheme. (B) qRT-PCR measurements of ACE-tRNA^{Arg} and ACE-tRNA^{Arg} barcode. (C) PTC suppression activity of ACE-tRNA^{Arg} and ACE-tRNA^{Arg}-barcode.

FIGS. 12A and 12B are photographs showing ArgTGA minicircle ligation products of different sizes and corresponding PCR products resolved on a 1.5% agarose gel containing ethidium bromide.

FIGS. 12C and 12D are photographs showing ArgTGA minicircle ligation products and PCR products that were incubated with T5 exonuclease and resolved on a 1.5% agarose gel containing ethidium bromide. The presence of exonuclease resistant products in the minicircle ligations indicates the production of covalently closed minicircle products.

FIGS. 13A, 13B, 13C, and 13D are a set of diagrams and photographs showing productions of: (FIG. 13A) a 200 bp CEDT product; (FIG. 13B) a 400 bp CEDT product; (FIG. 13C) a 900 bp CEDT/1× ArgTGA product; and (FIG. 13D) a 900 bp CEDT/4× ArgTGA product. Each of the corresponding PCR products was purified by anion exchange chromatography before being digested with N15 phage protelomerase (telN). The CEDT products displayed resistance to T5 exonuclease digest indicating the production of covalently closed ends by telN. Following endonuclease cleavage by the restriction enzyme Bsu36I, each CEDT product was susceptible to degradation by T5 exonuclease.

FIGS. 13E and 13F are a set of diagrams and photographs showing, respectively, a 850 bp 1× ArgTGA minicircle product and a 850 bp 4× ArgTGA minicircle product, both of which displayed resistance to T5 exonuclease digest indicating the production of covalently closed minicircles. Following endonuclease cleavage by the restriction enzyme SmaI, each minicircle product was susceptible to degradation by T5 exonuclease.

FIG. 14 is a set of diagrams showing that delivery of ACE-tRNAs as cDNA and RNA rescued endogenous CFTR mRNA in 16HBE14ge- cells.

FIGS. 15A and 15B are a set of diagrams showing a construct for generation of a fluorescent PTC reporter 16HBE14ge-cell line and uses of the cell line in PTC suppression assays.

FIGS. 16A and 16B are a set of diagrams showing that delivery of ACE-tRNAs as cDNA rescued endogenous CFTR mRNA in 16HBE14ge- cells. **=p<0.001; ****=p<0.0001.

FIGS. 17A and 17B are a set of diagrams showing that delivery of ACE-tRNAs in MC rescued endogenous R1162X-CFTR mRNA in 16HBE14ge- cells.

FIGS. 18A and 18B are a set of diagrams showing that delivery of ACE-tRNAs in MC rescued endogenous W1282X-CFTR mRNA in 16HBE14ge- cells with Leucine.

FIGS. 19A and 19B are a set of diagrams showing that delivery of ACE-tRNAs in different CEDTs rescued endogenous R1162X-CFTR mRNAs in 16HBE14ge- cells.

FIG. 20 is a diagram showing development of a PB-Donkey system.

FIGS. 21A and 21B are a set of diagrams showing that stable integration and expression of ACE-tRNA^{Arg} rescued endogenous CFTR function in R1162X 16HBE14ge-cells.

### DETAILED DESCRIPTION OF THE INVENTION

This invention relates to ACE-tRNAs, related vectors, and related delivery and uses for treatment of disorders associated with PTC or nonsense mutations.

About 10-15% of all genetic diseases are caused by nonsense mutations. In the United States alone, about 3 million people are affected, and it has been projected that one third of all inherited genetic disorders are nonsense-associated. There is a unifying mechanism for all of these diseases where a single nucleotide change converts an amino acid encoding codon to premature termination codon (TGA, TAG or TAA), resulting in truncated protein with complete loss of or altered function, and degradation of the mRNA transcript by nonsense mediated decay (NMD) pathways. The DNA molecules, pharmaceutical formulations, methods, and cells described herein can be used for treating these diseases.

In one aspect, this disclosure provides a closed end, circular, non-viral, and non-plasmid DNA molecule comprising (1) a promoter and (ii) a sequence encoding an anti-codon edited-tRNA (ACE-tRNA).

In some embodiments, the molecule is a closed end DNA thread (CEDT) molecule or a minicircle (MC) molecule.

In any of the above embodiments, the molecule further comprises one or more elements selected from the group consisting of a DNA nuclear targeting sequence (DTS), a transcription enhancing 5′ leader sequence (TELS), and an ACE-tRNA Barcoding Sequence (ABS).

In one embodiment, the DTS comprises a SV40-DTS.

In any of the above embodiments, the molecule is free of any bacterial nucleic acid sequence.

In any of the above embodiments, the molecule comprises 4, 3, 2, 1, or less CpG dinucleotides.

In one embodiment, the molecule is free of CpG dinucleotide.

In any of the above embodiments, the molecule is about 200 to about 1,000 bp in size.

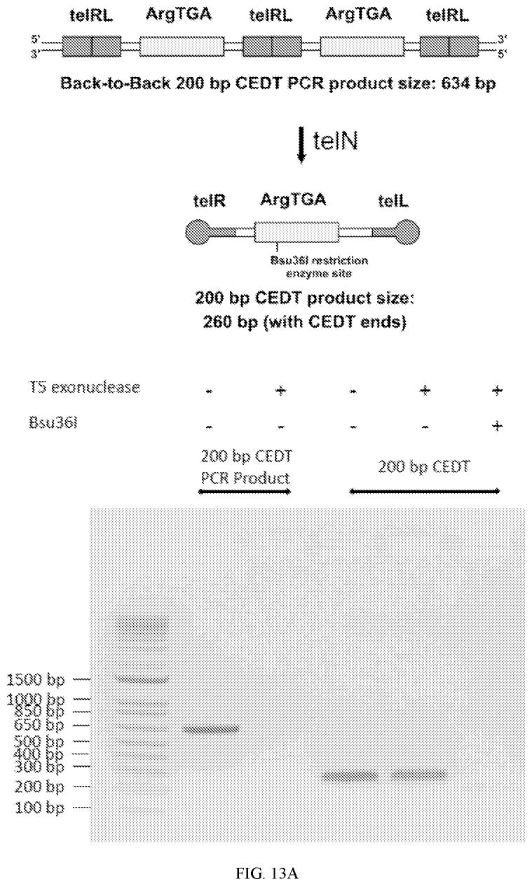

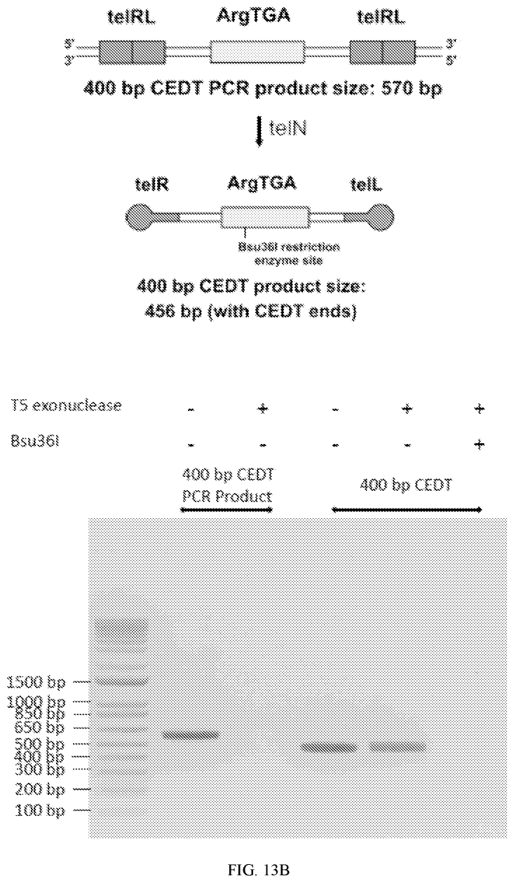

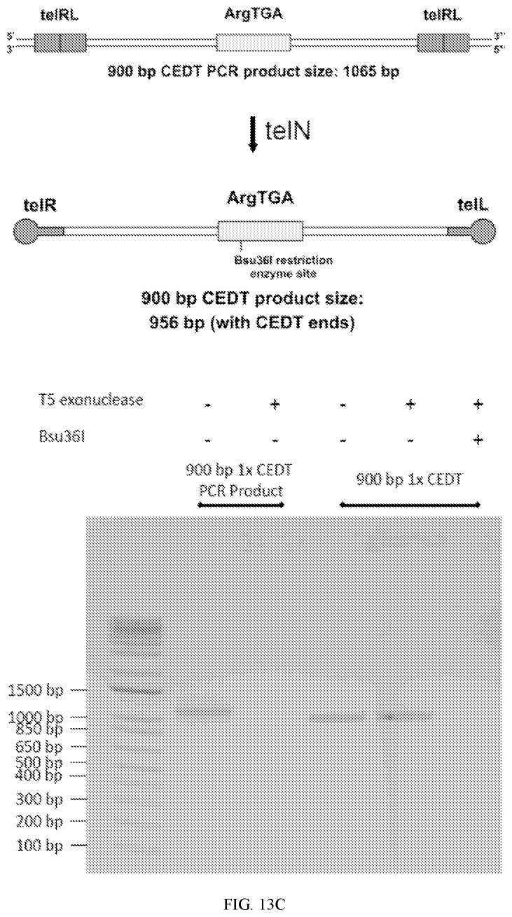

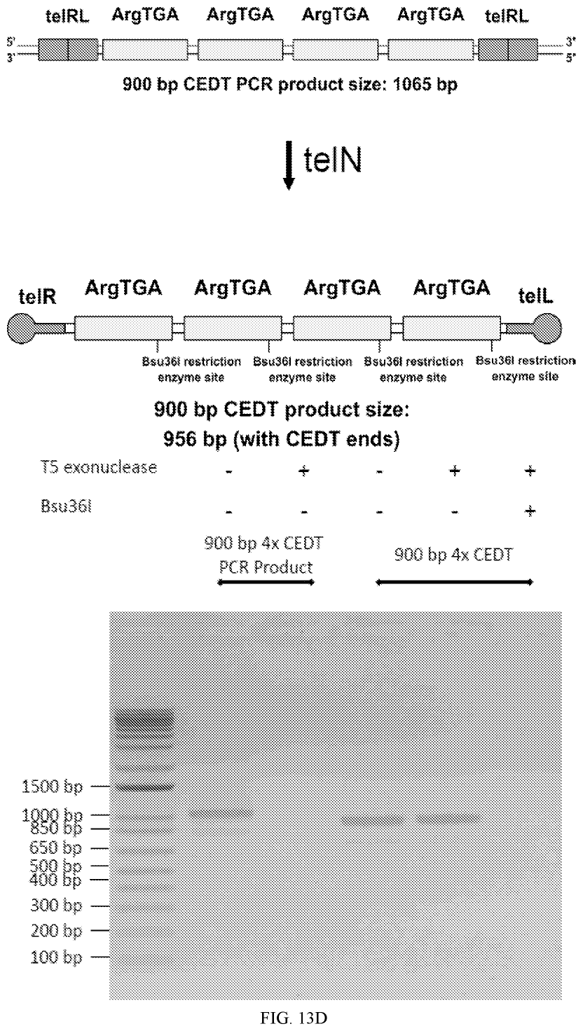

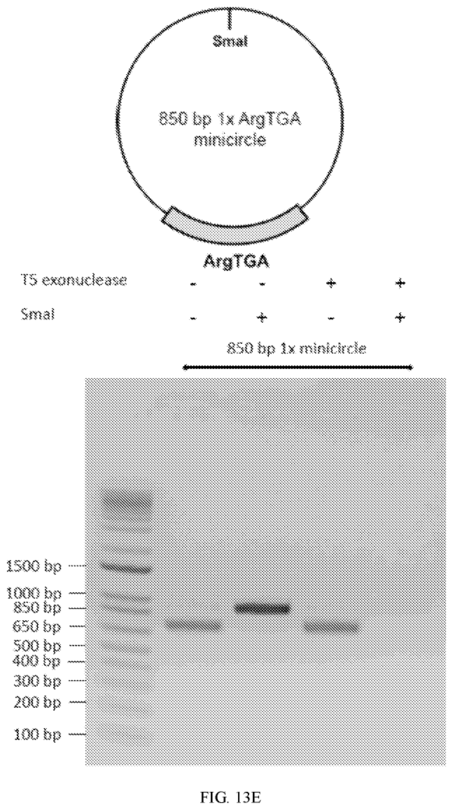

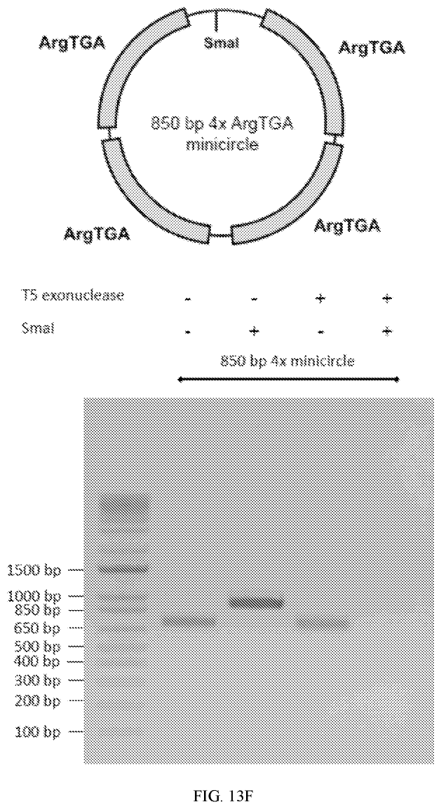

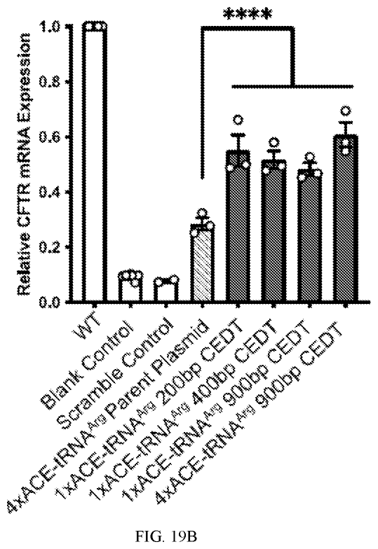

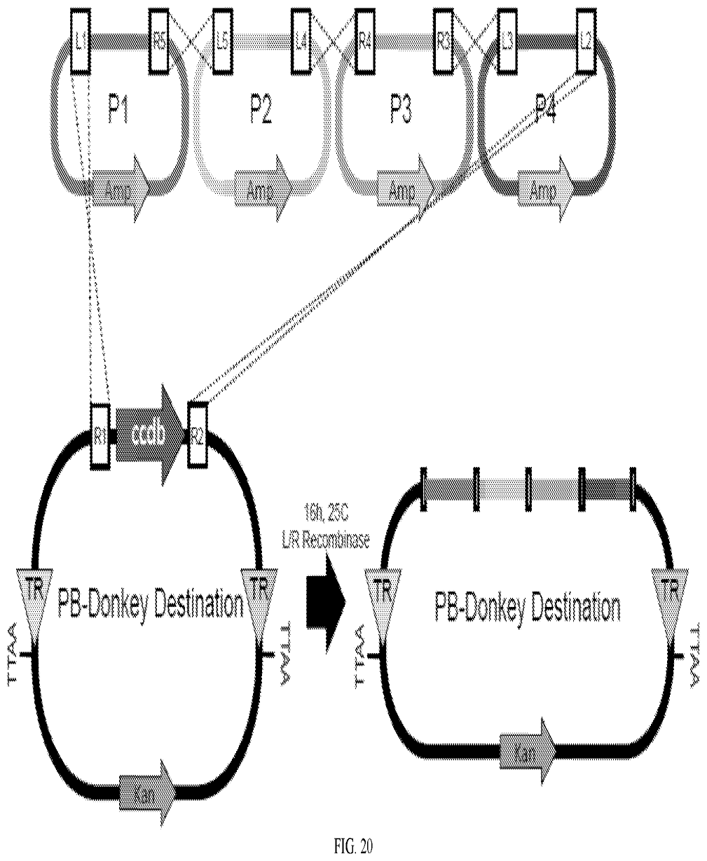

In one embodiment, the molecule is about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, or 1000 bp in size. In some examples of CEDTs, the ACE-tRNA-coding double stranded section are, e.g., about 200 bp, 400 bp, or 900 bp in size but the corresponding CEDTs are about 260 bp, 456 bp, or 956 bp in size due to added CEDT ends. In that case, the CEDTs are sometimes called 200 bp CEDT, 400 bp CEDT, or 900 bp CEDT to indicate the sizes of the ACE-tRNA-coding double stranded section. See, e.g., FIGS. 13 and 19.

In any of the above embodiments, the ACE-tRNA comprises a sequence (i) selected from the group consisting of SEQ ID NO: 1-10 or (ii) encoded by one selected from the group consisting of SEQ ID NO: 11-305.

In one embodiment, the ACE-tRNA comprises a sequence (i) selected from the group consisting of SEQ ID NO: 1, 4, 5, and 8 or (ii) encoded by one selected from the group consisting of SEQ ID NO: 79 and 94.

This disclosure also provides a pharmaceutical formulation comprising (i) the molecule of any one of the embodiments described above and (ii) a pharmaceutically acceptable carrier.

This disclosure further provides a method for expressing an ACE-tRNA in a cell, comprising (i) contacting the cell with the molecule of any the embodiments described above, and (ii) maintaining the cell under conditions permitting expression of the ACE-tRNA.

In one embodiment of the method, (i) the cell has a mutant nucleic acid comprising one or more premature termination codons (PTCs), (ii) the wild type of the mutant nucleic acid encodes a polypeptide, and (iii) the ACE-tRNA rescues the one or more PTCs and restores expression of the polypeptide.

In one embodiment, the polypeptide is cystic fibrosis transmembrane conductance regulator (CFTR) and the mutant nucleic acid encode a truncated CFTR.

In one embodiment, the mutant nucleic acid has a Trp-to-Stop PTC.

In one embodiment, the ACE-tRNA translates the Trp-to-Stop PTC into a Leu.

This disclosure further provides a host cell comprising the molecule of any one of the above described embodiments.

This disclosure further provides a method of treating a disease associated with a PTC in a subject in need thereof, the method comprising administering to the subject the molecule of any one of the above described embodiments or the pharmaceutical composition described above.

In some embodiments, the disease is selected from the group consisting of cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, McArdle's disease and Polycystic kidney disease.

In some embodiments, the disease is an ocular genetic disease selected from the group consisting of cone dystrophies, Stargardt's disease (STGD1), cone-rod dystrophy, retinitis pigmentosa (RP), increased susceptibility to age-related macular degeneration, Congenital stationary night blindness 2 (CSNB2), Congenital stationary night blindness 1 (CSNB1), Best Disease, VMD, and Leber congenital amaurosis (LCA16).

In some embodiments for the treatment method embodiments described above, the administering is carried out using nanoparticles, electroporation, polyethylenimine (PEI), receptor-targeted polyplexes, liposomes, or hydrodynamic injection.

### ACE-tRNAs

An ACE-tRNA is an engineered tRNA molecule whose sequence is engineered so that a PTC is effectively and therapeutically reverted back into the originally lost amino acid or a different amino acid. Such engineered tRNAs allow for “re-editing” of a disease-causing nonsense codon to a specific amino acid. As disclosed herein, an engineered tRNAs can target only one type of stop codon, such as TGA over TAC or TAA. The small size of these tRNA molecules makes them amenable to ready expression, as the tRNA and the promoter together can be only about 300 bp. To that end, an oligonucleotide can be synthesized to comprise the structural component of a tRNA gene functional in human cells. The sequence of this oligonucleotide can be designed based upon a known sequence with substitutions made in the anticodon region of the tRNA causing the specific tRNA to recognize a nonsense or other specific mutation. Examples of ACE-tRNAs include those described in WO2019090154, WO2019090169, and Lueck, J. D. et al. Nature communications 10, 822, 2019. The contents of each of these documents are incorporated by reference.

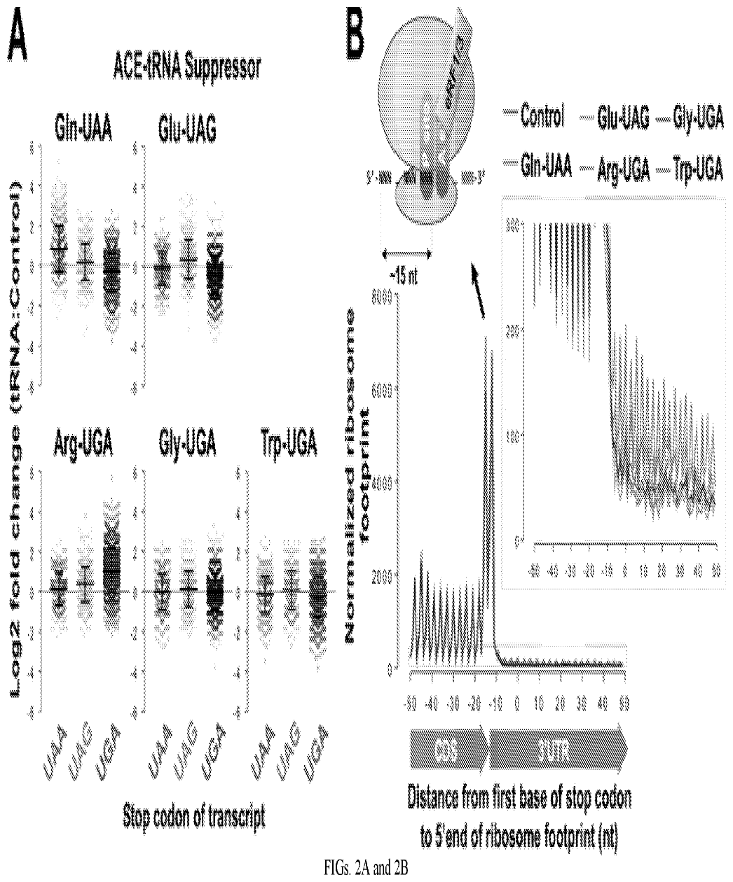

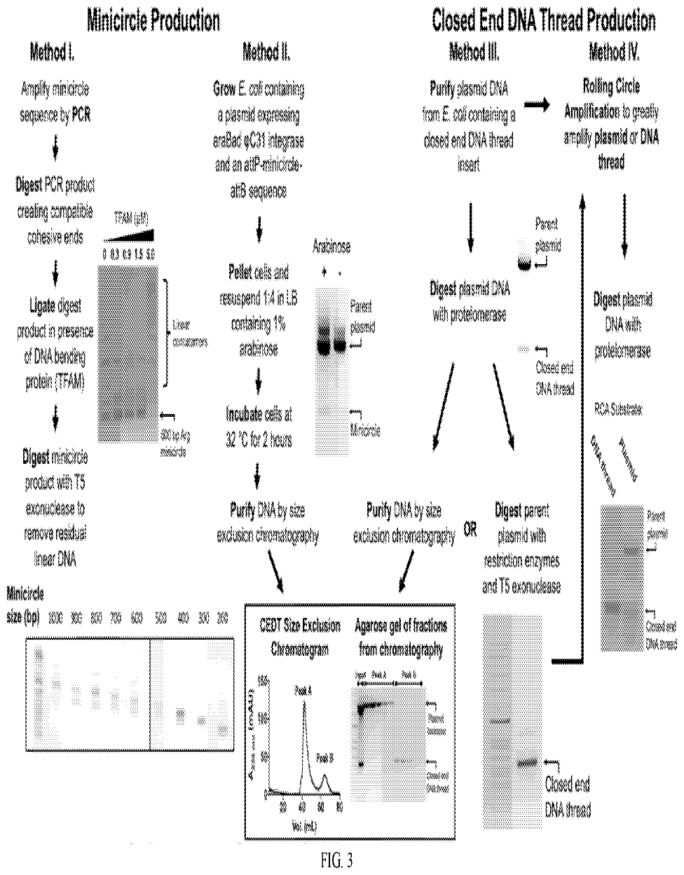

In general, an ACE-tRNA has a general four-arm structure comprising a T-arm, a D-arm, and anticodon-arm, and an acceptor arm (see FIG. 2 of WO2019090169). The T-arm is made up of a “T-stem” and a “TΨC loop.” In certain embodiments, the T-stem is modified to increase the stability of the tRNA. In certain embodiments, the ACE-tRNA has a modified T-stem that increases the biological activity to suppress stop sites relative to the endogenous T-stem sequence.

ACE-tRNAs can be used for suppression of PTCs. Yet, efficacious suppression of PTCs has potential drawbacks. For example, there was concern that a PTC suppression strategy could result in readthrough of real, native stop codons in vivo and readthrough of global native stop codons is deleterious. However, several cellular mechanisms are in place to limit both normal stop read-through and damaging effects thereof. More specifically, multiple in-frame stop codons are frequently found at normal translation termination, thus increasing the probability of translation termination in the presence of an efficient PTC suppressor. Furthermore, at least two cellular mechanisms are in place for the identification and degradation of proteins with erroneous translation termination, specialized ubiquitin ligases and ribosome associated pathway. There is evidence that natural stop codons at the end of genes have surrounding sequence landscapes that promote enhanced termination efficiency, and that termination complexes found at PTCs differ from those at “real” stops. Unexpectedly, discovery that endogenous stop codon read-through is common in animals and is not detrimental suggests that suppression of PTCs is a viable therapeutic approach. Indeed, ribosomal profiling preliminary data suggest that ACE-tRNA readthrough of “real” stops is infrequent (FIG. 2).

ACE-tRNAs useful for this invention can be made according to the strategy described in WO2019090154, WO2019090169, and Lueck, J. D. et al., Nature communications 10, 822 (2019). Using this strategy, an extensive library of ACE-tRNAs for effective rescue of PTCs in cell culture have been made. Below in Table 1 are some examples of ACE-tRNAs useful for this invention. Other engineered human tRNA sequences to suppress disease-causing PTCs include those described in WO2019090154, WO2019090169, and Lueck, J. D. et al., Nature communications 10, 822 (2019). Additional examples include those encoded by SEQ ID NOs: 11-305 in Table 2 below. In each of the sequences below, the three-letter sequence corresponding to the anti-codon is in small case and underlined.

TABLE 1

| | | | SEQ |
|---|---|---|---|
| | | | ID |
| NO. | tRNAscan-SE ID | Sequence | NO: |
| 1 | TrpTGAchr17.trna39 | GGCCUCGUGGCGCAACGGUAGCGCGUCUGACUucaG | 1 |
| | | AUCAGAAGGuUGCGUGUUCAAAUCACGUCGGGGUCA | |
| 22 | LeuTGAchr6.trna81 | ACCAGGAUGGCCGAGUGGUuAAGGCGUUGGACUuca | 2 |
| | | GAUCCAAUGGACAUAUGUCCGCGUGGGUUCGAACCC | |
| | | CACUCCUGGUA | |
| 23 | LeuTGAchr6.trna135 | ACCGGGAUGGCCGAGUGGUuAAGGCGUUGGACUuca | 3 |
| | | GAUCCAAUGGGCUGGUGCCCGCGUGGGUUCGAACCC | |
| | | CACUCUCGGUA | |
| 24 | LeuTGAchr11.trna4 | ACCAGAAUGGCCGAGUGGUuAAGGCGUUGGACUuca | 4 |
| | | GAUCCAAUGGAUUCAUAUCCGCGUGGGUUCGAACCC | |
| | | CACUUCUGGUA | |
| 17 | GlyTGAchr19.trna2 | GCGUUGGUGGUAUAGUGGUuAGCAUAGCUGCCUuca | 5 |
| | | AAGCAGUUGaCCCGGGUUCGAUUCCCGGCCAACGCA | |
| 18 | GlyTGAchr1.trna107 | GCGUUGGUGGUAUAGUGGUgAGCAUAGCUGCCUuca | 6 |
| | | AAGCAGUUGaCCCGGGUUCGAUUCCCGGCCAACGCA | |
| 19 | GlyTGAchr17.trna9 | GCGUUGGUGGUAUAGUGGUaAGCAUAGCUGCCUuca | 7 |
| | | AAGCAGUUGaCCCGGGUUCGAUUCCCGGCCAACGCA | |
| 21 | ArgTGAchr9.trna6/ | GGCUCUGUGGCGCAAUGGAuAGCGCAUUGGACUuca | 8 |
| | nointron | AAUUCAAAGGuUGUGGGUUCGAGUCCCACCAGAGUC | |
| | | G | |
| 18 | GlnTAGchr1.trna101 | GGUUCCAUGGUGUAAUGGUaAGCACUCUGGACUcua | 9 |
| | | AAUCCAGCGaUCCGAGUUCGAGUCUCGGUGGAACCU | |
| 27 | GlnTAGchr6.trna175 | GGCCCCAUGGUGUAAUGGUuAGCACUCUGGACUcua | 10 |
| | | AAUCCAGCGaUCCGAGUUCAAAUCUCGGUGGGACCU | |

TABLE 2

| | | | SEQ |
|---|---|---|---|
| | | | ID |
| NO. | tRNAscan-SE ID | Sequence | NO: |
| 1 | TrpTGAchr17.trna39 | GGCCTCGTGGCGCAACGGTAGCGCGTCTGACTtcaGA | 11 |
| | | TCAGAAGGtTGCGTGTTCAAATCACGTCGGGGTCA | |
| 2 | TrpTGAchr17.trna10 | GACCTCGTGGCGCAATGGTAGCGCGTCTGACTtcaGA | 12 |
| | | TCAGAAGGtTGCGTGTTCAAGTCACGTCGGGGTCA | |
| 3 | TrpTGAchr6.trna171 | GACCTCGTGGCGCAACGGTAGCGCGTCTGACTtcaGA | 13 |
| | | TCAGAAGGtTGCGTGTTCAAATCACGTCGGGGTCA | |
| 4 | TrpTGAchr12.trna6 | GACCTCGTGGCGCAACGGTAGCGCGTCTGACTtcaGA | 14 |
| | | TCAGAAGGCTGCGTGTTCGAATCACGTCGGGGTCA | |
| 5 | TrpTGAchr7.trna3 | GACCTCGTGGCGCAACGGCAGCGCGTCTGACTtcaGA | 15 |
| | | TCAGAAGGtTGCGTGTTCAAATCACGTCGGGGTCA | |
| 1 | TrpTAGchr17.trna39 | GGCCTCGTGGCGCAACGGTAGCGCGTCTGACTctaGA | 16 |
| | | TCAGAAGGtTGCGTGTTCAAATCACGTCGGGGTCA | |
| 2 | TrpTAGchr17.trna10 | GACCTCGTGGCGCAATGGTAGCGCGTCTGACTctaGA | 17 |
| | | TCAGAAGGtTGCGTGTTCAAGTCACGTCGGGGTCA | |
| 3 | TrpTAGchr6.trna171 | GACCTCGTGGCGCAACGGTAGCGCGTCTGACTctaGA | 18 |
| | | TCAGAAGGtTGCGTGTTCAAATCACGTCGGGGTCA | |
| 4 | TrpTAGchr12.trna6 | GACCTCGTGGCGCAACGGTAGCGCGTCTGACTctaGA | 19 |
| | | TCAGAAGGCTGCGTGTTCGAATCACGTCGGGGTCA | |
| 5 | TrpTAGchr7.trna3 | GACCTCGTGGCGCAACGGCAGCGCGTCTGACTctaGA | 20 |
| | | TCAGAAGGtTGCGTGTTCAAATCACGTCGGGGTCA | |
| 17 | GlyTGAchr19.trna2 | GCGTTGGTGGTATAGTGGTtAGCATAGCTGCCTtcaA | 21 |
| | | AGCAGTTGaCCCGGGTTCGATTCCCGGCCAACGCA | |
| 18 | GlyTGAchr1.trna107 | GCGTTGGTGGTATAGTGGTgAGCATAGCTGCCTtcaA | 22 |
| | | AGCAGTTGaCCCGGGTTCGATTCCCGGCCAACGCA | |
| 19 | GlyTGAchr17.trna9 | GCGTTGGTGGTATAGTGGTaAGCATAGCTGCCTtcaA | 23 |
| | | AGCAGTTGaCCCGGGTTCGATTCCCGGCCAACGCA | |
| 2 | ArgTGAchr3.trna8 | GGGCCAGTGGCGCAATGGAtAACGCGTCTGACTtcaG | 24 |
| | | ATCAGAAGAtTCTAGGTTCGACTCCTGGCTGGCTCG | |
| 3 | ArgTGAchr6.trna115 | GGCCGCGTGGCCTAATGGAtAAGGCGTCTGATTtcaG | 25 |
| | | ATCAGAAGAtTGAGGGTTCGAGTCCCTTCGTGGTCG | |
| 4 | ArgTGAchr17.trna21 | GACCCAGTGGCCTAATGGAtAAGGCATCAGCCTtcaG | 26 |
| | | AGCTGGGGAtTGTGGGTTCGAGTCCCATCTGGGTCG | |
| 5 | ArgTGAchr17.trna16 | GCCCCAGTGGCCTAATGGAtAAGGCACTGGCCTtcaA | 27 |
| | | AGCCAGGGAtTGTGGGTTCGAGTCCCACCTGGGGTA | |
| 6 | ArgTGAchr17.trna19 | GCCCCAGTGGCCTAATGGAtAAGGCACTGGCCTtcaA | 28 |
| | | AGCCAGGGAtTGTGGGTTCGAGTCCCACCTGGGGTG | |
| 7 | ArgTGAchr16.trna3 | GCCCCGGTGGCCTAATGGAtAAGGCATTGGCCTtcaA | 29 |
| | | AGCCAGGGAtTGTGGGTTCGAGTCCCACCCGGGGTA | |
| 8 | ArgTGAchr7.trna5 | GCCCCAGTGGCCTAATGGAtAAGGCATTGGCCTtcaA | 30 |
| | | AGCCAGGGAtTGTGGGTTCGAGTCCCATCTGGGGTG | |
| 9 | ArgTGAchr16.trna13 | GCCCCAGTGGCCTGATGGAtAAGGTACTGGCCTtcaA | 31 |
| | | AGCCAGGGAtTGTGGGTTCGAGTTCCACCTGGGGTA | |
| 10 | ArgTGAchr15.trna4 | GGCCGCGTGGCCTAATGGAtAAGGCGTCTGACTtcaG | 32 |
| | | ATCAGAAGAtTGCAGGTTCGAGTCCTGCCGCGGTCG | |
| 11 | ArgTGAchr6.trna4 | GACCACGTGGCCTAATGGAtAAGGCGTCTGACTtcaG | 33 |
| | | ATCAGAAGAtTGAGGGTTCGAATCCCTCCGTGGTTA | |
| 12 | ArgTGAchr17.trna17 | GACCGCGTGGCCTAATGGAtAAGGCGTCTGACTtcaG | 34 |
| | | ATCAGAAGAtTGAGGGTTCGAGTCCCTTCGTGGTCG | |
| 13 | ArgTGAchr6.trna3 | GACCACGTGGCCTAATGGAtAAGGCGTCTGACTtcaG | 35 |
| | | ATCAGAAGAtTGAGGGTTCGAATCCCTTCGTGGTTA | |
| 14 | ArgTGAchr6.trna125 | GACCACGTGGCCTAATGGAtAAGGCGTCTGACTtcaG | 36 |
| | | ATCAGAAGAtTGAGGGTTCGAATCCCTTCGTGGTTG | |
| 15 | ArgTGAchr9.trna5 | GGCCGTGTGGCCTAATGGAtAAGGCGTCTGACTtcaG | 37 |
| | | ATCAAAAGAtTGCAGGTTTGAGTTCTGCCACGGTCG | |
| 16 | ArgTGAchr1.trna10 | GGCTCCGTGGCGCAATGGAtAGCGCATTGGACTtcaA | 38 |
| | | gaggctgaaggcATTCAAAGGtTCCGGGTTCGAGTCC | |
| | | CGGCGGAGTCG | |
| 17 | ArgTGAchr1.trna10/ | GGCTCCGTGGCGCAATGGAtAGCGCATTGGACTtcaA | 39 |
| | nointron | ATTCAAAGGtTCCGGGTTCGAGTCCCGGCGGAGTCG | |
| 18 | ArgTGAchr17.trna3 | GGCTCTGTGGCGCAATGGAtAGCGCATTGGACTtcaA | 40 |
| | | gtgacgaatagagcaATTCAAAGGtTGTGGGTTCGAA | |
| | | TCCCACCAGAGTCG | |
| 19 | ArgTGAchr17.trna3/ | GGCTCTGTGGCGCAATGGAtAGCGCATTGGACTtcaA | 41 |
| | nointron | ATTCAAAGGtTGTGGGTTCGAATCCCACCAGAGTCG | |
| 20 | ArgTGAchr9.trna6 | GGCTCTGTGGCGCAATGGAtAGCGCATTGGACTtcaA | 42 |
| | | gctgagcctagtgtggtcATTCAAAGGtTGTGGGTTC | |
| | | GAGTCCCACCAGAGTCG | |
| 21 | ArgTGAchr9.trna6/ | GGCTCTGTGGCGCAATGGAtAGCGCATTGGACTtcaA | 43 |
| | | ATTCAAAGGtTGTGGGTTCGAGTCCCACCAGAGTCG | |
| 22 | ArgTGAchr11.trna3 | GGCTCTGTGGCGCAATGGAtAGCGCATTGGACTtcaA | 44 |
| | | gatagttagagaaATTCAAAGGtTGTGGGTTCGAGTC | |
| | | CCACCAGAGTCG | |
| 23 | ArgTGAchr1.trna79 | GTCTCTGTGGCGCAATGGAcgAGCGCGCTGGACTtca | 45 |
| | | AATCCAGAGGtTCCGGGTTCGAGTCCCGGCAGAGATG | |
| 25 | ArgTGAchr6.trna52/ | GGCTCTGTGGCGCAATGGAtAGCGCATTGGACTtcaA | 46 |
| | nointron | ATTCAAAGGtTGCGGGTTCGAGTCCCTCCAGAGTCG | |
| 14 | GlnTAGchr12.trna3 | GGTTCCATGGTGTAATGGTaAGCACCCTGGACTctaA | 47 |
| | | ATCCAGCAaCCAGAGTTCCAGTCTCAGCGtGGACCT | |
| 15 | GlnTAGchr5.trna23 | GGTAGTGTAGTCTACTGGTTAAACGCTTGGgCTctaA | 48 |
| | | CATTAAcGtCCTGGGTTCAAATCCCAGCTTTGTCA | |
| 16 | GlnTAGchr6.trna147 | GGTTCCATGGTGTAATGGTtAGCACTCTGGACTctaA | 49 |
| | | ATCCAGCGaTCCGAGTTCAAGTCTCGGTGGAACCT | |
| 17 | GlnTAGchr1.trna17 | GGTTCCATGGTGTAATGGTgAGCACTCTGGACTctaA | 50 |
| | | ATCCAGCGaTCCGAGTTCGAGTCTCGGTGGAACCT | |
| 18 | GlnTAGchr1.trna101 | GGTTCCATGGTGTAATGGTaAGCACTCTGGACTctaA | 51 |
| | | ATCCAGCGaTCCGAGTTCGAGTCTCGGTGGAACCT | |
| 19 | GlnTAGchr6.trna42 | GGTTCCATGGTGTAATGGTtAGCACTCTGGACTctaA | 52 |
| | | ATCCGGTAaTCCGAGTTCAAATCTCGGTGGAACCT | |
| 20 | GlnTAGchr6.trna132 | GGCCCCATGGTGTAATGGTcAGCACTCTGGACTctaA | 53 |
| | | ATCCAGCGaTCCGAGTTCAAATCTCGGTGGGACCC | |
| 21 | GlnTAGchr1.trna23 | GGTTCCATGGTGTAATGGTaAGCACTCTGGACTctaA | 54 |
| | | ATCCAGCCATCTGAGTTCGAGTCTCTGTGGAACCT | |
| 22 | GlnTAGchr1.trna111 | GGTTCCATGGTGTAATGGTgAGCACTTTGGACTctaA | 55 |
| | | ATACAGTGATCAGAGTTCAAGTCTCACTGGGACCT | |
| 23 | GlnTAGchr1.trna24 | GGTTCCATGgGTTAATGGTgAGCACCCTGGACTctaA | 56 |
| | | ATCAAGCGaTCCGAGTTCAAATCTCGGTGGTACCT | |
| 24 | GlnTAGchr19.trna4 | GTTTCCATGGTGTAATGGTgAGCACTCTGGACTctaA | 57 |
| | | ATCCAGAAATACATTCAAAGAATTAAGAACA | |
| 25 | GlnTAGchr17.trna14 | GGTCCCATGGTGTAATGGTtAGCACTCTGGACTctaA | 58 |
| | | ATCCAGCGaTCCGAGTTCAAATCTCGGTGGGACCT | |
| 26 | GlnTAGchr6.trna63 | GGTCCCATGGTGTAATGGTtAGCACTCTGGACTctaA | 59 |
| | | ATCCAGCAaTCCGAGTTCGAATCTCGGTGGGACCT | |
| 27 | GlnTAGchr6.trna175 | GGCCCCATGGTGTAATGGTtAGCACTCTGGACTctaA | 60 |
| | | ATCCAGCGaTCCGAGTTCAAATCTCGGTGGGACCT | |
| 28 | GlnTAGchr6.trna82 | GGTCCCATGGTGTAATGGTtAGCACTCTGGGCTctaA | 61 |
| | | ATCCAGCAaTCCGAGTTCGAATCTTGGTGGGACCT | |
| 29 | GlnTAGchr2.trna26 | GGCTGTGTACCTCAGTGGGCAAGGGTATGGACTctaA | 62 |
| | | AGCCAGACTaTTTGGGTTCAAATCCCAGCTTGGCCT | |
| 2 | GlnTAAnmt-tRNA-Gln | TAGGACATGGTGTGATAGGTAGCATGGAGAATTttaG | 63 |
| | chrx.trna1 | ATTCTCAGGGGTAGGTTCAATTCCTACAGTTCTAG | |
| 3 | GlnTAAnmt-tRNA-Glnc | TAGGACGTGGTGTGATAGGTAGCATGGGGAATTttaG | 64 |
| | hr7.trna32 | ATTCTCAGGGGTGGGTTCAATTCCTATAGTTCTAG | |
| 4 | GlnTAAnmt-tRNA-Gln | TAGGACGTGGTGTAGTAGGTAGCATGGAGAATGttaA | 65 |
| | chr7.trna7 | ATTCTCAGGGGTAGGTTCAATTCCTATAGTTCTAG | |
| 8 | GlnTAAnmt-tRNA-Gln | TCTAGGAtgTGGTGTGATAGGTAGCATGGAGAATTtt | 66 |
| | chr12.trna15 | aGATTCTCAGGGGTAGGTTCAATTCCTATaTTCTAGA | |
| | | A | |
| 9 | GlnTAAnmt-tRNA-Gln | TAGGACGTGGTGTGATAGGTAGCATGGAGAATTttaG | 67 |
| | chr2.trna21 | ATTCTCAGGGATGGGTTCAATTCCTATAGTCCTAG | |
| 10 | GlnTAAnmt-tRNA- | TAGGACGTGGTGTGATAGGTAGCACGGAGAATTttaG | 68 |
| | Glnchr2.trna9 | ATTCTCAGGGATGGGTTCAATTCCTGTAGTTCTAG | |
| 11 | GlnTAAchr6.trna1 | GGTTCCATGGTGTAATGGTtAGCACTCTGGACTttaA | 69 |
| | | ATCCAGCGaTCCGAGTTCAAATCTCGGTGGAACCT | |
| 16 | GlnTAAchr6.trna147 | GGTTCCATGGTGTAATGGTtAGCACTCTGGACTttaA | 70 |
| | | ATCCAGCGaTCCGAGTTCAAGTCTCGGTGGAACCT | |
| 17 | GlnTAAchr1.trna17 | GGTTCCATGGTGTAATGGTgAGCACTCTGGACTttaA | 71 |
| | | ATCCAGCGaTCCGAGTTCGAGTCTCGGTGGAACCT | |
| 18 | GlnTAAchr1.trna101 | GGTTCCATGGTGTAATGGTaAGCACTCTGGACTttaA | 72 |
| | | ATCCAGCGaTCCGAGTTCGAGTCTCGGTGGAACCT | |
| 19 | GlnTAAchr6.trna42 | GGTTCCATGGTGTAATGGTtAGCACTCTGGACTttaA | 73 |
| | | ATCCGGTAaTCCGAGTTCAAATCTCGGTGGAACCT | |
| 20 | GlnTAAchr6.trna132 | GGCCCCATGGTGTAATGGTcAGCACTCTGGACTttaA | 74 |
| | | ATCCAGCGaTCCGAGTTCAAATCTCGGTGGGACCC | |
| 21 | GlnTAAchr1.trna23 | GGTTCCATGGTGTAATGGTaAGCACTCTGGACTttaA | 75 |
| | | ATCCAGCCATCTGAGTTCGAGTCTCTGTGGAACCT | |
| 22 | GlnTAAchr1.trna111 | GGTTCCATGGTGTAATGGTgAGCACTTTGGACTttaA | 76 |
| | | ATACAGTGATCAGAGTTCAAGTCTCACTGGGACCT | |
| 23 | GlnTAAchr1.trna24 | GGTTCCATGgGTTAATGGTgAGCACCCTGGACTttaA | 77 |
| | | ATCAAGCGaTCCGAGTTCAAATCTCGGTGGTACCT | |
| 24 | GlnTAAchr19.trna4 | GTTTCCATGGTGTAATGGTgAGCACTCTGGACTttaA | 78 |
| | | ATCCAGAAATACATTCAAAGAATTAAGAACA | |
| 25 | GlnTAAchr17.trna14 | GGTCCCATGGTGTAATGGTtAGCACTCTGGACTttaA | 79 |
| | | ATCCAGCGaTCCGAGTTCAAATCTCGGTGGGACCT | |
| 26 | GlnTAAchr6.trna63 | GGTCCCATGGTGTAATGGTtAGCACTCTGGACTttaA | 80 |
| | | ATCCAGCAaTCCGAGTTCGAATCTCGGTGGGACCT | |
| 27 | GlnTAAchr6.trna175 | GGCCCCATGGTGTAATGGTtAGCACTCTGGACTttaA | 81 |
| | | ATCCAGCGaTCCGAGTTCAAATCTCGGTGGGACCT | |
| 28 | GlnTAAchr6.trna82 | GGTCCCATGGTGTAATGGTtAGCACTCTGGGCTttaA | 82 |
| | | ATCCAGCAaTCCGAGTTCGAATCTTGGTGGGACCT | |
| 29 | GlnTAAchr2.trna26 | GGCTGTGTACCTCAGTGGGCAAGGGTATGGACTttaA | 83 |
| | | AGCCAGACTaTTTGGGTTCAAATCCCAGCTTGGCCT | |
| 31 | GlnTAAchr8.trna10 | GGTACAGTGTTAAAGGGGagaAAAATTGCTGACTtta | 84 |
| | | AATaCAGTAGaCCTAGGTTTGAATCCTGGCTTTACCA | |
| 1 | GluTAAchr1.trna106 | TCCCTGGTGGTCTAGTGGTtAGGATTCGGCGCTttaA | 85 |
| | | CCGCCGCGGCCCGGGTTCGATTCCCGGTCAGGGAA | |
| 2 | GluTAAchr1.trna55 | TCCCTGGTGGTCTAGTGGTtAGGATTCGGCGCTttaA | 86 |
| | | CCGCCGCGGCCCGGGTTCGATTCCCGGTCAGGAAA | |
| 5 | GluTAAchr2.trna18 | TCCCATATGGTCTAGCGGTtAGGATTCCTGGTTttaA | 87 |
| | | CCCAGGTGGCCCGGGTTCGACTCCCGGTATGGGAA | |
| 8 | GluTAAchr13.trna2 | TCCCACATGGTCTAGCGGTtAGGATTCCTGGTTttaA | 88 |
| | | CCCAGGCGGCCCGGGTTCGACTCCCGGTGTGGGAA | |
| 9 | GluTAAchr1.trna5 | TCCCTGGTGGTCTAGTGGCtAGGATTCGGCGCTttaA | 89 |
| | | CCGCCGCGGCCCGGGTTCGATTCCCGGCCAGGGAA | |
| 10 | GluTAAchr1.trna123 | TCCCTGGTGGTCTAGTGGCtAGGATTCGGCGCTttaA | 90 |
| | | CCGCCGCGGCCCGGGTTCGATTCCCGGTCAGGGAA | |
| 1 | GluTAGchr1.trna106 | TCCCTGGTGGTCTAGTGGTtAGGATTCGGCGCTctaA | 91 |
| | | CCGCCGCGGCCCGGGTTCGATTCCCGGTCAGGGAA | |
| 5 | GluTAGchr2.trna18 | TCCCATATGGTCTAGCGGTtAGGATTCCTGGTTctaA | 92 |
| | | CCCAGGTGGCCCGGGTTCGACTCCCGGTATGGGAA | |
| 6 | GluTAGchr1.trna92 | TCCGTGGTGGTCTAGTGGCtAGGATTCGGCGCTctaA | 93 |
| | | CCGCCTGCAGCTCGAGTTCGATTCCTGGTCAGGGAA | |
| 8 | GluTAGchr13.trna2 | TCCCACATGGTCTAGCGGTtAGGATTCCTGGTTctaA | 94 |
| | | CCCAGGCGGCCCGGGTTCGACTCCCGGTGTGGGAA | |
| 9 | GluTAGchr1.trna5 | TCCCTGGTGGTCTAGTGGCtAGGATTCGGCGCTctaA | 95 |
| | | CCGCCGCGGCCCGGGTTCGATTCCCGGCCAGGGAA | |
| 10 | GluTAGchr1.trna123 | TCCCTGGTGGTCTAGTGGCtAGGATTCGGCGCTctaA | 96 |
| | | CCGCCGCGGCCCGGGTTCGATTCCCGGTCAGGGAA | |
| 14 | GluTAGchr1.trna86 | TCCCTGGTGGTCTAGTGGCtAGGATTCGGCGCTctaA | 97 |
| | | CCGCCTGCAGCTCGAGTTCGATTCCTGGTCAGGGAA | |
| 7 | TyrTAAchr6.trna14 | CCTTCGATAGCTCAGTTGGTAGAGCGGAGGACTttaG | 98 |
| | | ttggctgtgtccttagacATCCTTAGGtCGCTGGTTC | |
| | | GAATCCGGCTCGAAGGA | |
| 8 | TyrTAAchr6.trna14/ | CCTTCGATAGCTCAGTTGGTAGAGCGGAGGACTttaG | 99 |
| | nointron | ATCCTTAGGtCGCTGGTTCGAATCCGGCTCGAAGGA | |
| 9 | TyrTAAchr7.trna12 | GGGGGTATAGCTCAGGGCtAGAGCTtTTTGACTttaG | 100 |
| | | AGCAAGAGGtCCCTGGTTCAAATCCAGGTTCTCCCT | |
| 10 | TyrTAAchr7.trna28 | TATAGCTCAGTGGTAGAGCATTTAACTttaGATCAAG | 101 |
| | | AGGtCCCTGGATCAACTCTGGGTG | |
| 11 | TyrTAAchr15.trna6 | GTCAGTGTTGCACAACGGTtaAGTGAAGAGGCTttaA | 102 |
| | | ACCCAGACTGGATGGGTTCAATTCCCATCTCTGCCG | |
| 12 | TyrTAAchr2.trna2 | CCTTCGATAGCTCAGTTGGTAGAGCGGAGGACTttaG | 103 |
| | | tggatagggcgtggcaATCCTTAGGtCGCTGGTTCGA | |
| | | TTCCGGCTCGAAGGA | |
| 13 | TyrTAAchr2.trna2/ | CCTTCGATAGCTCAGTTGGTAGAGCGGAGGACTttaG | 104 |
| | nointron | ATCCTTAGGtCGCTGGTTCGATTCCGGCTCGAAGGA | |
| 14 | TyrTAAchr6.trna16 | CCTTCGATAGCTCAGTTGGTAGAGCGGAGGACTttaG | 105 |
| | | gctcattaagcaaggtATCCTTAGGtCGCTGGTTCGA | |
| | | ATCCGGCTCGGAGGA | |
| 15 | TyrTAAchr6.trna16/ | CCTTCGATAGCTCAGTTGGTAGAGCGGAGGACTttaG | 106 |
| | nointron | ATCCTTAGGtCGCTGGTTCGAATCCGGCTCGGAGGA | |
| 16 | TyrTAAchr14.trna19 | CCTTCGATAGCTCAGCTGGTAGAGCGGAGGACTttaG | 107 |
| | | attgtatagacatttgcggacATCCTTAGGtCGCTGG | |
| | | TTCGATTCCAGCTCGAAGGA | |
| 17 | TyrTAAchr14.trna19/ | CCTTCGATAGCTCAGCTGGTAGAGCGGAGGACTttaG | 108 |
| | nointron | ATCCTTAGGtCGCTGGTTCGATTCCAGCTCGAAGGA | |
| 18 | TyrTAAchr8.trna2 | CCTTCGATAGCTCAGCTGGTAGAGCGGAGGACTttaG | 109 |
| | | ctacttcctcagcaggagacATCCTTAGGtCGCTGGT | |
| | | TCGATTCCGGCTCGAAGGA | |
| 19 | TyrTAAchr8.trna2/ | CCTTCGATAGCTCAGCTGGTAGAGCGGAGGACTttaG | 110 |
| | nointron | ATCCTTAGGtCGCTGGTTCGATTCCGGCTCGAAGGA | |
| 20 | TyrTAAchr8.trna3 | CCTTCGATAGCTCAGCTGGTAGAGCGGAGGACTttaG | 111 |
| | | gcgcgcgcccgtggccATCCTTAGGtCGCTGGTTCGA | |
| | | TTCCGGCTCGAAGGA | |
| 21 | TyrTAAchr8.trna3/ | CCTTCGATAGCTCAGCTGGTAGAGCGGAGGACTttaG | 112 |
| | nointron | ATCCTTAGGtCGCTGGTTCGATTCCGGCTCGAAGGA | |
| 22 | TyrTAAchr14.trna20 | CCTTCGATAGCTCAGCTGGTAGAGCGGAGGACTttaG | 113 |
| | | cctgtagaaacatttgtggacATCCTTAGGtCGCTGG | |
| | | TTCGATTCCGGCTCGAAGGA | |
| 23 | TyrTAAchr14.trna20/ | CCTTCGATAGCTCAGCTGGTAGAGCGGAGGACTttaG | 114 |
| | nointron | ATCCTTAGGtCGCTGGTTCGATTCCGGCTCGAAGGA | |
| 24 | TyrTAAchr14.trna17 | CCTTCGATAGCTCAGCTGGTAGAGCGGAGGACTttaG | 115 |
| | | attgtacagacatttgcggacATCCTTAGGtCGCTGG | |
| | | TTCGATTCCGGCTCGAAGGA | |
| 25 | TyrTAAchr14.trna17/ | CCTTCGATAGCTCAGCTGGTAGAGCGGAGGACTttaG | 116 |
| | nointron | ATCCTTAGGtCGCTGGTTCGATTCCGGCTCGAAGGA | |
| 26 | TyrTAAchr14.trna5 | CCTTCGATAGCTCAGCTGGTAGAGCGGAGGACTttaG | 117 |
| | | tacttaatgtgtggtcATCCTTAGGtCGCTGGTTCGA | |
| | | TTCCGGCTCGAAGGA | |
| 27 | TyrTAAchr14.trna5/ | CCTTCGATAGCTCAGCTGGTAGAGCGGAGGACTttaG | 118 |
| | nointron | ATCCTTAGGtCGCTGGTTCGATTCCGGCTCGAAGGA | |
| 28 | TyrTAAchr6.trna17 | CCTTCGATAGCTCAGCTGGTAGAGCGGAGGACTttaG | 119 |
| | | gggtttgaatgtggtcATCCTTAGGtCGCTGGTTCGA | |
| | | ATCCGGCTCGGAGGA | |
| 29 | TyrTAAchr6.trna17/ | CCTTCGATAGCTCAGCTGGTAGAGCGGAGGACTttaG | 120 |
| | nointron | ATCCTTAGGtCGCTGGTTCGAATCCGGCTCGGAGGA | |
| 30 | TyrTAAchr14.trna18 | CCTTCGATAGCTCAGCTGGTAGAGCGGAGGACTttaG | 121 |
| | | actgcggaaacgtttgtggacATCCTTAGGtCGCTGG | |
| | | TTCAATTCCGGCTCGAAGGA | |
| 31 | TyrTAAchr14.trna18/ | CCTTCGATAGCTCAGCTGGTAGAGCGGAGGACTttaG | 122 |
| | nointron | ATCCTTAGGtCGCTGGTTCAATTCCGGCTCGAAGGA | |
| 32 | TyrTAAchr6.trna15 | CTTTCGATAGCTCAGTTGGTAGAGCGGAGGACTttaG | 123 |
| | | gttcattaaactaaggcATCCTTAGGtCGCTGGTTCG | |
| | | AATCCGGCTCGAAGGA | |
| 33 | TyrTAAchr6.trna15/ | CTTTCGATAGCTCAGTTGGTAGAGCGGAGGACTttaG | 124 |
| | nointron | ATCCTTAGGtCGCTGGTTCGAATCCGGCTCGAAGGA | |
| 34 | TyrTAAchr8.trna11 | TCTTCAATAGCTCAGCTGGTAGAGCGGAGGACTttaG | 125 |
| | | gtgcacgcccgtggccATTCTTAGGTGCTGGTTTGAT | |
| | | TCCGACTTGGAGAG | |
| 40 | TyrTAAchr7.trna1 | GGTAAAATGGCTGAGTAAGCATTAGACTttaAATCTA | 126 |
| | | AAGaCAGAGGTCAAGGCCTCTTTTTTCCT | |
| 4 | TyrTAGchr1.trna52 | GGTAAAATGACTGAGTAAGCATTAGACTctaAATCTA | 127 |
| | | AAGaCAGAGGTCAAGACCTCTTTTTACCA | |
| 5 | TyrTAGchr11.trna9 | GGTAAAATGGCTGAGTAAGCATTAGACTctaAATCTA | 128 |
| | | AAGaCAGAGGTCAAGGCCTCTTTTTACCA | |
| 7 | TyrTAGchr6.trna14 | CCTTCGATAGCTCAGTTGGTAGAGCGGAGGACTctaG | 129 |
| | | ttggctgtgtccttagacATCCTTAGGtCGCTGGTTC | |
| | | GAATCCGGCTCGAAGGA | |
| 8 | TyrTAGchr6.trna14/ | CCTTCGATAGCTCAGTTGGTAGAGCGGAGGACTctaG | 130 |
| | nointron | ATCCTTAGGtCGCTGGTTCGAATCCGGCTCGAAGGA | |
| 9 | TyrTAGchr7.trna12 | GGGGGTATAGCTCAGGGCtAGAGCTtTTTGACTctaG | 131 |
| | | AGCAAGAGGtCCCTGGTTCAAATCCAGGTTCTCCCT | |
| 10 | TyrTAGchr7.trna28 | TATAGCTCAGTGGTAGAGCATTTAACTctaGATCAAG | 132 |
| | | AGGtCCCTGGATCAACTCTGGGTG | |
| 11 | TyrTAGchr15.trna6 | GTCAGTGTTGCACAACGGTtaAGTGAAGAGGCTctaA | 133 |
| | | ACCCAGACTGGATGGGTTCAATTCCCATCTCTGCCG | |
| 12 | TyrTAGchr2.trna2 | CCTTCGATAGCTCAGTTGGTAGAGCGGAGGACTctaG | 134 |
| | | tggatagggcgtggcaATCCTTAGGtCGCTGGTTCGA | |
| | | TTCCGGCTCGAAGGA | |
| 13 | TyrTAGchr2.trna2/ | CCTTCGATAGCTCAGTTGGTAGAGCGGAGGACTctaG | 135 |
| | nointron | ATCCTTAGGtCGCTGGTTCGATTCCGGCTCGAAGGA | |
| 14 | TyrTAGchr6.trna16 | CCTTCGATAGCTCAGTTGGTAGAGCGGAGGACTctaG | 136 |
| | | gctcattaagcaaggtATCCTTAGGtCGCTGGTTCGA | |
| | | ATCCGGCTCGGAGGA | |
| 15 | TyrTAGchr6.trna16/ | CCTTCGATAGCTCAGTTGGTAGAGCGGAGGACTctaG | 137 |
| | nointron | ATCCTTAGGtCGCTGGTTCGAATCCGGCTCGGAGGA | |
| 16 | TyrTAGchr14.trna19 | CCTTCGATAGCTCAGCTGGTAGAGCGGAGGACTctaG | 138 |
| | | attgtatagacatttgcggacATCCTTAGGtCGCTGG | |
| | | TTCGATTCCAGCTCGAAGGA | |
| 17 | TyrTAG | CCTTCGATAGCTCAGCTGGTAGAGCGGAGGACTctaG | 139 |
| | chr14.trna19/nointron | ATCCTTAGGtCGCTGGTTCGATTCCAGCTCGAAGGA | |
| 18 | TyrTAGchr8.trna2 | CCTTCGATAGCTCAGCTGGTAGAGCGGAGGACTctaG | 140 |
| | | ctacttcctcagcaggagacATCCTTAGGtCGCTGGT | |
| | | TCGATTCCGGCTCGAAGGA | |
| 19 | TyrTAGchr8.trna2/ | CCTTCGATAGCTCAGCTGGTAGAGCGGAGGACTctaG | 141 |
| | nointron | ATCCTTAGGtCGCTGGTTCGATTCCGGCTCGAAGGA | |
| 20 | TyrTAGchr8.trna3 | CCTTCGATAGCTCAGCTGGTAGAGCGGAGGACTctaG | 142 |
| | | gcgcgcgcccgtggccATCCTTAGGtCGCTGGTTCGA | |
| | | TTCCGGCTCGAAGGA | |
| 21 | TyrTAGchr8.trna3/ | CCTTCGATAGCTCAGCTGGTAGAGCGGAGGACTctaG | 143 |
| | nointron | ATCCTTAGGtCGCTGGTTCGATTCCGGCTCGAAGGA | |
| 22 | TyrTAGchr14.trna20 | CCTTCGATAGCTCAGCTGGTAGAGCGGAGGACTctaG | 144 |
| | | cctgtagaaacatttgtggacATCCTTAGGtCGCTGG | |
| | | TTCGATTCCGGCTCGAAGGA | |
| 23 | TyrTAGchr14.trna20/ | CCTTCGATAGCTCAGCTGGTAGAGCGGAGGACTctaG | 145 |
| | nointron | ATCCTTAGGtCGCTGGTTCGATTCCGGCTCGAAGGA | |
| 24 | TyrTAGchr14.trna17 | CCTTCGATAGCTCAGCTGGTAGAGCGGAGGACTctaG | 146 |
| | | attgtacagacatttgcggacATCCTTAGGtCGCTGG | |
| | | TTCGATTCCGGCTCGAAGGA | |
| 25 | TyrTAGchr14.trna17/ | CCTTCGATAGCTCAGCTGGTAGAGCGGAGGACTctaG | 147 |
| | nointron | ATCCTTAGGtCGCTGGTTCGATTCCGGCTCGAAGGA | |
| 26 | TyrTAGchr14.trna5 | CCTTCGATAGCTCAGCTGGTAGAGCGGAGGACTctaG | 148 |
| | | tacttaatgtgtggtcATCCTTAGGtCGCTGGTTCGA | |
| | | TTCCGGCTCGAAGGA | |
| 27 | TyrTAGchr14.trna5/ | CCTTCGATAGCTCAGCTGGTAGAGCGGAGGACTctaG | 149 |
| | nointron | ATCCTTAGGtCGCTGGTTCGATTCCGGCTCGAAGGA | |
| 28 | TyrTAGchr6.trna17 | CCTTCGATAGCTCAGCTGGTAGAGCGGAGGACTctaG | 150 |
| | | gggtttgaatgtggtcATCCTTAGGtCGCTGGTTCGA | |
| | | ATCCGGCTCGGAGGA | |
| 29 | TyrTAGchr6.trna17/ | CCTTCGATAGCTCAGCTGGTAGAGCGGAGGACTctaG | 151 |
| | nointron | ATCCTTAGGtCGCTGGTTCGAATCCGGCTCGGAGGA | |
| 30 | TyrTAGchr14.trna18 | CCTTCGATAGCTCAGCTGGTAGAGCGGAGGACTctaG | 152 |
| | | actgcggaaacgtttgtggacATCCTTAGGtCGCTGG | |
| | | TTCAATTCCGGCTCGAAGGA | |
| 31 | TyrTAGchr14.trna18/ | CCTTCGATAGCTCAGCTGGTAGAGCGGAGGACTctaG | 153 |
| | nointron | ATCCTTAGGtCGCTGGTTCAATTCCGGCTCGAAGGA | |
| 32 | TyrTAGchr6.trna15 | CTTTCGATAGCTCAGTTGGTAGAGCGGAGGACTctaG | 154 |
| | | gttcattaaactaaggcATCCTTAGGtCGCTGGTTCG | |
| | | AATCCGGCTCGAAGGA | |
| 33 | TyrTAGchr6.trna15/ | CTTTCGATAGCTCAGTTGGTAGAGCGGAGGACTctaG | 155 |
| | nointron | ATCCTTAGGtCGCTGGTTCGAATCCGGCTCGAAGGA | |
| 34 | TyrTAGchr8.trna11 | TCTTCAATAGCTCAGCTGGTAGAGCGGAGGACTctaG | 156 |
| | | gtgcacgcccgtggccATTCTTAGGTGCTGGTTTGAT | |
| | | TCCGACTTGGAGAG | |
| 3 | LeuTAAchr6.trna77 | GGTAGCGTGGCCGAGCGGTctAAGGCGCTGGATTtta | 157 |
| | | GCTCCAGTCTCTTCGGGGGCGTGGGTTCAAATCCCAC | |
| | | CGCTGCCA | |
| 9 | LeuTAAchr6.trna100/ | GTCAGGATGGCCGAGTGGTctAAGGCGCCAGACTtta | 158 |
| | nointron | GTTCTGGTCTCCAATGGAGGCGTGGGTTCGAATCCCA | |
| | | CTTCTGACA | |
| 10 | LeuTAAchr6.trna73 | GTCAGGATGGCCGAGTGGTctAAGGCGCCAGACTtta | 159 |
| | | GcttggcttcctcgtgttgaggaTTCTGGTCTCCAAT | |
| | | GGAGGCGTGGGTTCGAATCCCACTTCTGACA | |
| 11 | LeuTAAchr6.trna73/ | GTCAGGATGGCCGAGTGGTctAAGGCGCCAGACTtta | 160 |
| | nointron | GTTCTGGTCTCCAATGGAGGCGTGGGTTCGAATCCCA | |
| | | CTTCTGACA | |
| 12 | LeuTAAchr6.trna141 | GTCAGGATGGCCGAGTGGTctAAGGCGCCAGACTtta | 161 |
| | | GcttactgcttcctgtgttcgggtcTTCTGGTCTCCG | |
| | | TATGGAGGCGTGGGTTCGAATCCCACTTCTGACA | |
| 13 | LeuTAAchr6.trna141/ | GTCAGGATGGCCGAGTGGTctAAGGCGCCAGACTtta | 162 |
| | nointron | GTTCTGGTCTCCGTATGGAGGCGTGGGTTCGAATCCC | |
| | | ACTTCTGACA | |
| 14 | LeuTAAchr6.trna142 | GTCAGGATGGCCGAGTGGTctAAGGCGCCAGACTtta | 163 |
| | | GttgctacttcccaggtttggggcTTCTGGTCTCCGC | |
| | | ATGGAGGCGTGGGTTCGAATCCCACTTCTGACA | |
| 15 | LeuTAAchr6.trna142/ | GTCAGGATGGCCGAGTGGTctAAGGCGCCAGACTtta | 164 |
| | nointron | GTTCTGGTCTCCGCATGGAGGCGTGGGTTCGAATCCC | |
| | | ACTTCTGACA | |
| 16 | LeuTAAchr1.trna54 | GTCAGGATGGCCGAGTGGTctAAGGCGCCAGACTtta | 165 |
| | | GgtaagcaccttgcctgcgggctTTCTGGTCTCCGGA | |
| | | TGGAGGCGTGGGTTCGAATCCCACTTCTGACA | |
| 18 | LeuTAAchr11.trna1 | GCCTCCTTAGTGCAGTAGGTAGCGCATCAGTCTttaA | 166 |
| | | ATCTGAATGgtCCTGAGTTCAAGCCTCAGAGGGGGCA | |
| 22 | LeuTAAchr6.trna81 | ACCAGGATGGCCGAGTGGTtAAGGCGTTGGACTttaG | 167 |
| | | ATCCAATGGACATATGTCCGCGTGGGTTCGAACCCCA | |
| | | CTCCTGGTA | |
| 23 | LeuTAAchr6.trna135 | ACCGGGATGGCCGAGTGGTtAAGGCGTTGGACTttaG | 168 |
| | | ATCCAATGGGCTGGTGCCCGCGTGGGTTCGAACCCCA | |
| | | CTCTCGGTA | |
| 24 | LeuTAAchr11.trna4 | ACCAGAATGGCCGAGTGGTtAAGGCGTTGGACTttaG | 169 |
| | | ATCCAATGGATTCATATCCGCGTGGGTTCGAACCCCA | |
| | | CTTCTGGTA | |
| 25 | LeuTAAchr6.trna156 | ACCGGGATGGCTGAGTGGTtAAGGCGTTGGACTttaG | 170 |
| | | ATCCAATGGACAGGTGTCCGCGTGGGTTCGAGCCCCA | |
| | | CTCCCGGTA | |
| 26 | LeuTAAchr6.trna79 | ACTCATTTGGCTGAGTGGTtAAGGCATTGGACTttaG | 171 |
| | | ATCCAATGGAGTAGTGGCTGTGTGGGTTTAAACCCCA | |
| | | CTACTGGTA | |
| 27 | LeuTAAchr1.trna9 | GAGAAAGTCATCGTAGTTACGAAGTTGGCTttaACCC | 172 |
| | | AGTTTtGGGAGGTTCAATTCCTTCCTTTCTCT | |
| 28 | LeuTAAchr11.trna12 | ACCAGGATGGCCAAGTAGTTaAAGGCACTGGACTtta | 173 |
| | | GAGCCAATGGACATATGTCTGTGTGGGTTTGAACCCC | |
| | | ACTCCTGGTG | |
| 29 | LeuTAAchr17.trna42 | GGTAGCGTGGCCGAGCGGTctAAGGCGCTGGATTtta | 174 |
| | | GCTCCAGTCTCTTCGGAGGCGTGGGTTCGAATCCCAC | |
| | | CGCTGCCA | |
| 30 | LeuTAAchr14.trna2 | GGTAGTGTGGCCGAGCGGTctAAGGCGCTGGATTtta | 175 |
| | | GCTCCAGTCTCTTCGGGGGCGTGGGTTCGAATCCCAC | |
| | | CACTGCCA | |
| 31 | LeuTAAchr16.trna27 | GGTAGCGTGGCCGAGTGGTctAAGGCGCTGGATTtta | 176 |
| | | GCTCCAGTCATTTCGATGgCGTGGGTTCGAATCCCAC | |
| | | CGCTGCCA | |
| 32 | LeuTAAchr14.trna16 | GGTAGTGTGGTTGAATGGTctAAGGCACTGAATTtta | 177 |
| | | GCTCCAGTCTCTTTGGGGaCGTGGGTTTAAATCCCAC | |
| | | TGCTGCAA | |
| 1 | LeuTAGchr4.trna2 | GTTAAGATGGCAGAGCCtGGTaATTGCActaAACTTA | 178 |
| | | AAATTTTATAAtCAGAGGTTCAACTCCTCTTCTTAAC | |
| | | A | |
| 2 | LeuTAGnmtchrX.trna2 | GTTAAGATGGCAGAGCCCGGCaATTGCActaGACTTA | 179 |
| | | AAACTTTATAAtCAGAGGTTCAACTCCTCTCATTAAC | |
| | | A | |
| 3 | LeuTAGchr6.trna77 | GGTAGCGTGGCCGAGCGGTctAAGGCGCTGGATTcta | 180 |
| | | GCTCCAGTCTCTTCGGGGGCGTGGGTTCAAATCCCAC | |
| | | CGCTGCCA | |
| 4 | LeuTAGchr6.trna127 | GGTAGCGTGGCCGAGTGGTctAAGACGCTGGATTcta | 181 |
| | | GCTCCAGTCTCTTCGGGGGCGTGGGTTTGAATCCCAC | |
| | | CGCTGCCA | |
| 7 | LeuTAGchr5.trna20 | GCCGAGCGGTctAAGGCTCCGGATTctaGCGCCGGTG | 182 |
| | | TCTTCGGAGgCATGGGTTCGAATTCCAC | |
| 8 | LeuTAGchr6.trna100 | GTCAGGATGGCCGAGTGGTctAAGGCGCCAGACTcta | 183 |
| | | GctaagcttcctccgcggtggggaTTCTGGTCTCCAA | |
| | | TGGAGGCGTGGGTTCGAATCCCACTTCTGACA | |
| 9 | LeuTAGchr6.trna100/ | GTCAGGATGGCCGAGTGGTctAAGGCGCCAGACTcta | 184 |
| | nointron | GTTCTGGTCTCCAATGGAGGCGTGGGTTCGAATCCCA | |
| | | CTTCTGACA | |
| 10 | LeuTAGchr6.trna73 | GTCAGGATGGCCGAGTGGTctAAGGCGCCAGACTcta | 185 |
| | | GcttggcttcctcgtgttgaggaTTCTGGTCTCCAAT | |
| | | GGAGGCGTGGGTTCGAATCCCACTTCTGACA | |
| 11 | LeuTAGchr6.trna73/ | GTCAGGATGGCCGAGTGGTctAAGGCGCCAGACTcta | 186 |
| | nointron | GTTCTGGTCTCCAATGGAGGCGTGGGTTCGAATCCCA | |
| | | CTTCTGACA | |
| 12 | LeuTAGchr6.trna141 | GTCAGGATGGCCGAGTGGTctAAGGCGCCAGACTcta | 187 |
| | | GcttactgcttcctgtgttcgggtcTTCTGGTCTCCG | |
| | | TATGGAGGCGTGGGTTCGAATCCCACTTCTGACA | |
| 13 | LeuTAGchr6.trna141/ | GTCAGGATGGCCGAGTGGTctAAGGCGCCAGACTcta | 188 |
| | nointron | GTTCTGGTCTCCGTATGGAGGCGTGGGTTCGAATCCC | |
| | | ACTTCTGACA | |
| 14 | LeuTAGchr6.trna142 | GTCAGGATGGCCGAGTGGTctAAGGCGCCAGACTcta | 189 |
| | | GttgctacttcccaggtttggggcTTCTGGTCTCCGC | |
| | | ATGGAGGCGTGGGTTCGAATCCCACTTCTGACA | |
| 15 | LeuTAGchr6.trna142/ | GTCAGGATGGCCGAGTGGTctAAGGCGCCAGACTcta | 190 |
| | nointron | GTTCTGGTCTCCGCATGGAGGCGTGGGTTCGAATCCC | |
| | | ACTTCTGACA | |
| 16 | LeuTAGchr1.trna54 | GTCAGGATGGCCGAGTGGTctAAGGCGCCAGACTcta | 191 |
| | | GgtaagcaccttgcctgcgggctTTCTGGTCTCCGGA | |
| | | TGGAGGCGTGGGTTCGAATCCCACTTCTGACA | |
| 18 | LeuTAGchr11.trna1 | GCCTCCTTAGTGCAGTAGGTAGCGCATCAGTCTctaA | 192 |
| | | ATCTGAATGgtCCTGAGTTCAAGCCTCAGAGGGGGCA | |
| 22 | LeuTAGchr6.trna81 | ACCAGGATGGCCGAGTGGTtAAGGCGTTGGACTctaG | 193 |
| | | ATCCAATGGACATATGTCCGCGTGGGTTCGAACCCCA | |
| | | CTCCTGGTA | |
| 23 | LeuTAGchr6.trna135 | ACCGGGATGGCCGAGTGGTtAAGGCGTTGGACTctaG | 194 |
| | | ATCCAATGGGCTGGTGCCCGCGTGGGTTCGAACCCCA | |
| | | CTCTCGGTA | |
| 24 | LeuTAGchr11.trna4 | ACCAGAATGGCCGAGTGGTtAAGGCGTTGGACTctaG | 195 |
| | | ATCCAATGGATTCATATCCGCGTGGGTTCGAACCCCA | |
| | | CTTCTGGTA | |
| 25 | LeuTAGchr6.trna156 | ACCGGGATGGCTGAGTGGTtAAGGCGTTGGACTctaG | 196 |
| | | ATCCAATGGACAGGTGTCCGCGTGGGTTCGAGCCCCA | |
| | | CTCCCGGTA | |
| 26 | LeuTAGchr6.trna79 | ACTCATTTGGCTGAGTGGTtAAGGCATTGGACTctaG | 197 |
| | | ATCCAATGGAGTAGTGGCTGTGTGGGTTTAAACCCCA | |
| | | CTACTGGTA | |
| 29 | LeuTAGchr17.trna42 | GGTAGCGTGGCCGAGCGGTctAAGGCGCTGGATTcta | 198 |
| | | GCTCCAGTCTCTTCGGAGGCGTGGGTTCGAATCCCAC | |
| | | CGCTGCCA | |
| 30 | LeuTAGchr14.trna2 | GGTAGTGTGGCCGAGCGGTctAAGGCGCTGGATTcta | 199 |
| | | GCTCCAGTCTCTTCGGGGGCGTGGGTTCGAATCCCAC | |
| | | CACTGCCA | |
| 31 | LeuTAGchr16.trna27 | GGTAGCGTGGCCGAGTGGTctAAGGCGCTGGATTcta | 200 |
| | | GCTCCAGTCATTTCGATGgCGTGGGTTCGAATCCCAC | |
| | | CGCTGCCA | |
| 32 | LeuTAGchr14.trna16 | GGTAGTGTGGTTGAATGGTctAAGGCACTGAATTcta | 201 |
| | | GCTCCAGTCTCTTTGGGGaCGTGGGTTTAAATCCCAC | |
| | | TGCTGCAA | |
| 3 | LeuTGAchr6.trna77 | GGTAGCGTGGCCGAGCGGTctAAGGCGCTGGATTtca | 202 |
| | | GCTCCAGTCTCTTCGGGGGCGTGGGTTCAAATCCCAC | |
| | | CGCTGCCA | |
| 8 | LeuTGAchr6.trna100 | GTCAGGATGGCCGAGTGGTctAAGGCGCCAGACTtca | 203 |
| | | GctaagcttcctccgcggtggggaTTCTGGTCTCCAA | |
| | | TGGAGGCGTGGGTTCGAATCCCACTTCTGACA | |
| 9 | LeuTGAchr6.trna100/ | GTCAGGATGGCCGAGTGGTctAAGGCGCCAGACTtca | 204 |
| | nointron | GTTCTGGTCTCCAATGGAGGCGTGGGTTCGAATCCCA | |
| | | CTTCTGACA | |
| 10 | LeuTGAchr6.trna73 | GTCAGGATGGCCGAGTGGTctAAGGCGCCAGACTtca | 205 |
| | | GcttggcttcctcgtgttgaggaTTCTGGTCTCCAAT | |
| | | GGAGGCGTGGGTTCGAATCCCACTTCTGACA | |
| 11 | LeuTGAchr6.trna73/ | GTCAGGATGGCCGAGTGGTctAAGGCGCCAGACTtca | 206 |
| | nointron | GTTCTGGTCTCCAATGGAGGCGTGGGTTCGAATCCCA | |
| | | CTTCTGACA | |
| 12 | LeuTGAchr6.trna141 | GTCAGGATGGCCGAGTGGTctAAGGCGCCAGACTtca | 207 |
| | | GcttactgcttcctgtgttcgggtcTTCTGGTCTCCG | |
| | | TATGGAGGCGTGGGTTCGAATCCCACTTCTGACA | |
| 13 | LeuTGAchr6.trna141/ | GTCAGGATGGCCGAGTGGTctAAGGCGCCAGACTtca | 208 |
| | nointron | GTTCTGGTCTCCGTATGGAGGCGTGGGTTCGAATCCC | |
| | | ACTTCTGACA | |
| 14 | LeuTGAchr6.trna142 | GTCAGGATGGCCGAGTGGTctAAGGCGCCAGACTtca | 209 |
| | | GttgctacttcccaggtttggggcTTCTGGTCTCCGC | |
| | | ATGGAGGCGTGGGTTCGAATCCCACTTCTGACA | |
| 15 | LeuTGAchr6.trna142/ | GTCAGGATGGCCGAGTGGTctAAGGCGCCAGACTtca | 210 |
| | nointron | GTTCTGGTCTCCGCATGGAGGCGTGGGTTCGAATCCC | |
| | | ACTTCTGACA | |
| 16 | LeuTGAchr1.trna54 | GTCAGGATGGCCGAGTGGTctAAGGCGCCAGACTtca | 211 |
| | | GgtaagcaccttgcctgcgggctTTCTGGTCTCCGGA | |
| | | TGGAGGCGTGGGTTCGAATCCCACTTCTGACA | |
| 22 | LeuTGAchr6.trna81 | ACCAGGATGGCCGAGTGGTtAAGGCGTTGGACTtcaG | 212 |
| | | ATCCAATGGACATATGTCCGCGTGGGTTCGAACCCCA | |
| | | CTCCTGGTA | |
| 23 | LeuTGAchr6.trna135 | ACCGGGATGGCCGAGTGGTtAAGGCGTTGGACTtcaG | 213 |
| | | ATCCAATGGGCTGGTGCCCGCGTGGGTTCGAACCCCA | |
| | | CTCTCGGTA | |
| 24 | LeuTGAchr11.trna4 | ACCAGAATGGCCGAGTGGTtAAGGCGTTGGACTtcaG | 214 |
| | | ATCCAATGGATTCATATCCGCGTGGGTTCGAACCCCA | |
| | | CTTCTGGTA | |
| 25 | LeuTGAchr6.trna156 | ACCGGGATGGCTGAGTGGTtAAGGCGTTGGACTtcaG | 215 |
| | | ATCCAATGGACAGGTGTCCGCGTGGGTTCGAGCCCCA | |
| | | CTCCCGGTA | |
| 26 | LeuTGAchr6.trna79 | ACTCATTTGGCTGAGTGGTtAAGGCATTGGACTtcaG | 216 |
| | | ATCCAATGGAGTAGTGGCTGTGTGGGTTTAAACCCCA | |
| | | CTACTGGTA | |
| 29 | LeuTGAchr17.trna42 | GGTAGCGTGGCCGAGCGGTctAAGGCGCTGGATTtca | 217 |
| | | GCTCCAGTCTCTTCGGAGGCGTGGGTTCGAATCCCAC | |
| | | CGCTGCCA | |
| 30 | LeuTGAchr14.trna2 | GGTAGTGTGGCCGAGCGGTctAAGGCGCTGGATTtca | 218 |
| | | GCTCCAGTCTCTTCGGGGGCGTGGGTTCGAATCCCAC | |
| | | CACTGCCA | |
| 31 | LeuTGAchr16.trna27 | GGTAGCGTGGCCGAGTGGTctAAGGCGCTGGATTtca | 219 |
| | | GCTCCAGTCATTTCGATGgCGTGGGTTCGAATCCCAC | |
| | | CGCTGCCA | |
| 2 | SerTAGnmtchr2.trna7 | GAGAAGGTCATAGAGGTtATGGGATTGGCTctaAACC | 220 |
| | | AGTCTCTGGGGGGTTCGATTCCCTCCTTTTTCA | |
| 5 | SerTAGchr6.trna148 | GTAGTCGTGGCCGAGTGGTtAAGGCGATGGACTctaA | 221 |
| | | ATCCATTGGGGTTTCCCCGCGCAGGTTCGAATCCTGC | |
| | | CGACTACG | |
| 6 | SerTAGchr6.trna50 | GTAGTCGTGGCCGAGTGGTtAAGGCGATGGACTctaA | 222 |
| | | ATCCATTGGGGTTTCCCCACGCAGGTTCGAATCCTGC | |
| | | CGACTACG | |
| 7 | SerTAGchr6.trna146 | GTAGTCGTGGCCGAGTGGTtAAGGTGATGGACTctaA | 223 |
| | | ACCCATTGGGGTCTCCCCGCGCAGGTTCGAATCCTGC | |
| | | CGACTACG | |
| 8 | SerTAGchr7.trna15 | GGGTGTATGGCTCAGGGGTAGAGAATTTGACTctaGA | 224 |
| | | TCAAGAGGtCCCTGGTTCAAATCCAGGTGCCCCCT | |
| 9 | SerTAGchr11.trna10 | AGTTGTAGCTGAGTGGTtAAGGCAACGAGCTctaAAT | 225 |
| | | TCGTTGGTTTCTCTCTgTGCAGGTTTGAATCCTGCTA | |
| | | ATTA | |
| 10 | SerTAGchr11.trna8 | CAAGAAATTCATAGAGGTTATGGGATTGGCTctaAAC | 226 |
| | | CAGTTTCAGGAGGTTCGATTCCTTCCTTTTTGG | |
| 12 | SerTAGchr6.trna34 | GCTGTGATGGCCGAGTGGTtAAGGCGTTGGACTctaA | 227 |
| | | ATCCAATGGGGTCTCCCCGCGCAGGTTCAAATCCTGC | |
| | | TCACAGCG | |
| 13 | SerTAGchr6.trna138 | GCTGTGATGGCCGAGTGGTtAAGGTGTTGGACTctaA | 228 |
| | | ATCCAATGGGGGTTCCCCGCGCAGGTTCAAATCCTGC | |
| | | TCACAGCG | |
| 14 | SerTAGchr12.trna2 | GTCACGGTGGCCGAGTGGTtAAGGCGTTGGACTctaA | 229 |
| | | ATCCAATGGGGTTTCCCCGCACAGGTTCGAATCCTGT | |
| | | TCGTGACG | |
| 15 | SerTAGchr6.trna30 | GACGAGGTGGCCGAGTGGTtAAGGCGATGGACTctaA | 230 |
| | | ATCCATTGTGCTCTGCACGCGTGGGTTCGAATCCCAC | |
| | | CCTCGTCG | |
| 16 | SerTAGchr6.trna43 | GACGAGGTGGCCGAGTGGTtAAGGCGATGGACTctaA | 231 |
| | | ATCCATTGTGCTCTGCACGCGTGGGTTCGAATCCCAC | |
| | | CTTCGTCG | |
| 17 | SerTAGchr11.trna6 | GGCCGGTTAGCTCAGTTGGTtAGAGCGTGCTctaACT | 232 |
| | | AATGCCAGGGtCGAGGTTTCGATCCCCGTACGGGCCT | |
| 18 | SerTAGchr6.trna61 | GACGAGGTGGCCGAGTGGTtAAGGCGATGGACTctaA | 233 |
| | | ATCCATTGTGCTCTGCACACGTGGGTTCGAATCCCAT | |
| | | CCTCGTCG | |
| 19 | SerTAGchr6.trna176 | GAGGCCTGGCCGAGTGGTtAAGGCGATGGACTctaAA | 234 |
| | | TCCATTGTGCTCTGCACGCGTGGGTTCGAATCCCATC | |
| | | CTCG | |
| 20 | SerTAGchr10.trna2 | GCAGCGATGGCCGAGTGGTtAAGGCGTTGGACTctaA | 235 |
| | | ATCCAATGGGGTCTCCCCGCGCAGGTTCGAACCCTGC | |
| | | TCGCTGCG | |
| 21 | SerTAGchr6.trna51 | GTAGTCGTGGCCGAGTGGTtAAGGCGATGGACTctaA | 236 |
| | | ATCCATTGGGGTTTCCCCGCGCAGGTTCGAATCCTGC | |
| | | CGACTACG | |
| 22 | SerTAGchr6.trna173 | GTAGTCGTGGCCGAGTGGTtAAGGCGATGGACTctaA | 237 |
| | | ATCCATTGGGGTCTCCCCGCGCAGGTTCGAATCCTGC | |
| | | CGACTACG | |
| 23 | SerTAGchr6.trna149 | GTAGTCGTGGCCGAGTGGTtAAGGCGATGGACTctaA | 238 |
| | | ATCCATTGGGGTTTCCCCGCGCAGGTTCGAATCCTGT | |
| | | CGGCTACG | |
| 5 | SerTGAchr6.trna148 | GTAGTCGTGGCCGAGTGGTtAAGGCGATGGACTtcaA | 239 |
| | | ATCCATTGGGGTTTCCCCGCGCAGGTTCGAATCCTGC | |
| | | CGACTACG | |
| 6 | SerTGAchr6.trna50 | GTAGTCGTGGCCGAGTGGTtAAGGCGATGGACTtcaA | 240 |
| | | ATCCATTGGGGTTTCCCCACGCAGGTTCGAATCCTGC | |
| | | CGACTACG | |
| 7 | SerTGAchr6.trna146 | GTAGTCGTGGCCGAGTGGTtAAGGTGATGGACTtcaA | 241 |
| | | ACCCATTGGGGTCTCCCCGCGCAGGTTCGAATCCTGC | |
| | | CGACTACG | |
| 9 | SerTGAchr11.trna10 | AGTTGTAGCTGAGTGGTtAAGGCAACGAGCTtcaAAT | 242 |
| | | TCGTTGGTTTCTCTCTgTGCAGGTTTGAATCCTGCTA | |
| | | ATTA | |
| 11 | SerTGAchr17.trna41 | GCTGTGATGGCCGAGTGGTtAAGGCGTTGGACTtcaA | 243 |
| | | ATCCAATGGGGTCTCCCCGCGCAGGTTCGAATCCTGC | |
| | | TCACAGCG | |
| 12 | SerTGAchr6.trna34 | GCTGTGATGGCCGAGTGGTtAAGGCGTTGGACTtcaA | 244 |
| | | ATCCAATGGGGTCTCCCCGCGCAGGTTCAAATCCTGC | |
| | | TCACAGCG | |
| 13 | SerTGAchr6.trna138 | GCTGTGATGGCCGAGTGGTtAAGGTGTTGGACTtcaA | 245 |
| | | ATCCAATGGGGGTTCCCCGCGCAGGTTCAAATCCTGC | |
| | | TCACAGCG | |
| 14 | SerTGAchr12.trna2 | GTCACGGTGGCCGAGTGGTtAAGGCGTTGGACTtcaA | 246 |
| | | ATCCAATGGGGTTTCCCCGCACAGGTTCGAATCCTGT | |
| | | TCGTGACG | |
| 15 | SerTGAchr6.trna30 | GACGAGGTGGCCGAGTGGTtAAGGCGATGGACTtcaA | 247 |
| | | ATCCATTGTGCTCTGCACGCGTGGGTTCGAATCCCAC | |
| | | CCTCGTCG | |
| 16 | SerTGAchr6.trna43 | GACGAGGTGGCCGAGTGGTtAAGGCGATGGACTtcaA | 248 |
| | | ATCCATTGTGCTCTGCACGCGTGGGTTCGAATCCCAC | |
| | | CTTCGTCG | |
| 17 | SerTGAchr11.trna6 | GGCCGGTTAGCTCAGTTGGTtAGAGCGTGCTtcaACT | 249 |
| | | AATGCCAGGGtCGAGGTTTCGATCCCCGTACGGGCCT | |
| 18 | SerTGAchr6.trna61 | GACGAGGTGGCCGAGTGGTtAAGGCGATGGACTtcaA | 250 |
| | | ATCCATTGTGCTCTGCACACGTGGGTTCGAATCCCAT | |
| | | CCTCGTCG | |
| 20 | SerTGAchr10.trna2 | GCAGCGATGGCCGAGTGGTtAAGGCGTTGGACTtcaA | 251 |
| | | ATCCAATGGGGTCTCCCCGCGCAGGTTCGAACCCTGC | |
| | | TCGCTGCG | |
| 21 | SerTGAchr6.trna51 | GTAGTCGTGGCCGAGTGGTtAAGGCGATGGACTtcaA | 252 |
| | | ATCCATTGGGGTTTCCCCGCGCAGGTTCGAATCCTGC | |
| | | CGACTACG | |
| 22 | SerTGAchr6.trna173 | GTAGTCGTGGCCGAGTGGTtAAGGCGATGGACTtcaA | 253 |
| | | ATCCATTGGGGTCTCCCCGCGCAGGTTCGAATCCTGC | |
| | | CGACTACG | |
| 23 | SerTGAchr6.trna149 | GTAGTCGTGGCCGAGTGGTtAAGGCGATGGACTtcaA | 254 |
| | | ATCCATTGGGGTTTCCCCGCGCAGGTTCGAATCCTGT | |
| | | CGGCTACG | |
| 5 | SerTAAchr6.trna148 | GTAGTCGTGGCCGAGTGGTtAAGGCGATGGACTttaA | 255 |
| | | ATCCATTGGGGTTTCCCCGCGCAGGTTCGAATCCTGC | |
| | | CGACTACG | |
| 6 | SerTAAchr6.trna50 | GTAGTCGTGGCCGAGTGGTtAAGGCGATGGACTttaA | 256 |
| | | ATCCATTGGGGTTTCCCCACGCAGGTTCGAATCCTGC | |
| | | CGACTACG | |
| 7 | SerTAAchr6.trna146 | GTAGTCGTGGCCGAGTGGTtAAGGTGATGGACTttaA | 257 |
| | | ACCCATTGGGGTCTCCCCGCGCAGGTTCGAATCCTGC | |
| | | CGACTACG | |
| 11 | SerTAAchr17.trna41 | GCTGTGATGGCCGAGTGGTtAAGGCGTTGGACTttaA | 258 |
| | | ATCCAATGGGGTCTCCCCGCGCAGGTTCGAATCCTGC | |
| | | TCACAGCG | |
| 12 | SerTAAchr6.trna34 | GCTGTGATGGCCGAGTGGTtAAGGCGTTGGACTttaA | 259 |
| | | ATCCAATGGGGTCTCCCCGCGCAGGTTCAAATCCTGC | |
| | | TCACAGCG | |
| 13 | SerTAAchr6.trna138 | GCTGTGATGGCCGAGTGGTtAAGGTGTTGGACTttaA | 260 |
| | | ATCCAATGGGGGTTCCCCGCGCAGGTTCAAATCCTGC | |
| | | TCACAGCG | |
| 14 | SerTAAchr12.trna2 | GTCACGGTGGCCGAGTGGTtAAGGCGTTGGACTttaA | 261 |
| | | ATCCAATGGGGTTTCCCCGCACAGGTTCGAATCCTGT | |
| | | TCGTGACG | |
| 15 | SerTAAchr6.trna30 | GACGAGGTGGCCGAGTGGTtAAGGCGATGGACTttaA | 262 |
| | | ATCCATTGTGCTCTGCACGCGTGGGTTCGAATCCCAC | |
| | | CCTCGTCG | |
| 16 | SerTAAchr6.trna43 | GACGAGGTGGCCGAGTGGTtAAGGCGATGGACTttaA | 263 |
| | | ATCCATTGTGCTCTGCACGCGTGGGTTCGAATCCCAC | |
| | | CTTCGTCG | |
| 17 | SerTAAchr11.trna6 | GGCCGGTTAGCTCAGTTGGTtAGAGCGTGCTttaACT | 264 |
| | | AATGCCAGGGtCGAGGTTTCGATCCCCGTACGGGCCT | |
| 18 | SerTAAchr6.trna61 | GACGAGGTGGCCGAGTGGTtAAGGCGATGGACTttaA | 265 |
| | | ATCCATTGTGCTCTGCACACGTGGGTTCGAATCCCAT | |
| | | CCTCGTCG | |
| 19 | SerTAAchr6.trna176 | GAGGCCTGGCCGAGTGGTtAAGGCGATGGACTttaAA | 266 |
| | | TCCATTGTGCTCTGCACGCGTGGGTTCGAATCCCATC | |
| | | CTCG | |
| 20 | SerTAAchr10.trna2 | GCAGCGATGGCCGAGTGGTtAAGGCGTTGGACTttaA | 267 |
| | | ATCCAATGGGGTCTCCCCGCGCAGGTTCGAACCCTGC | |
| | | TCGCTGCG | |
| 21 | SerTAAchr6.trna51 | GTAGTCGTGGCCGAGTGGTtAAGGCGATGGACTttaA | 268 |
| | | ATCCATTGGGGTTTCCCCGCGCAGGTTCGAATCCTGC | |
| | | CGACTACG | |
| 22 | SerTAAchr6.trna173 | GTAGTCGTGGCCGAGTGGTLAAGGCGATGGACTttaA | 269 |
| | | ATCCATTGGGGTCTCCCCGCGCAGGTTCGAATCCTGC | |
| | | CGACTACG | |
| 23 | SerTAAchr6.trna149 | GTAGTCGTGGCCGAGTGGTtAAGGCGATGGACTttaA | 270 |
| | | ATCCATTGGGGTTTCCCCGCGCAGGTTCGAATCCTGT | |
| | | CGGCTACG | |
| 1 | LysTAAchr19.trna6 | GCCCAGCTAGCTCAGTCGGTAGAGCATAAGACTttaA | 271 |
| | | ATCTCAGGGtTGTGGATTCGTGCCCCATGCTGGGTG | |
| 8 | LysTAAchr16.trna31 | GCCCGGCTAGCTCAGTCGGTAGAGCATGAGACCttaA | 272 |
| | | ATCTCAGGGtCGTGGGTTCGAGCCCCACGTTGGGCG | |
| 9 | LysTAAchr16.trna11 | GCCCGGCTAGCTCAGTCGGTAGAGCATGGGACTttaA | 273 |
| | | ATCTCAGGGtCGTGGGTTCGAGCCCCACGTTGGGCG | |
| 10 | LysTAAchr16.trna30 | GCCCGGCTAGCTCAGTCGATAGAGCATGAGACTttaA | 274 |
| | | ATCTCAGGGtCGTGGGTTCGAGCCGCACGTTGGGCG | |
| 12 | LysTAAchr16.trna6 | GCCTGGCTAGCTCAGTCGGCAAAGCATGAGACTttaA | 275 |
| | | ATCTCAGGGtCGTGGGCTCGAGCTCCATGTTGGGCG | |
| 16 | LysTAAchr16.trna23 | GCCTGGATAGCTCAGTTGGTAGAGCATCAGACTttaA | 276 |
| | | ATCTGAGGGtCCAGGGTTCAAGTCCCTGTTCAGGCA | |
| 19 | LysTAAchr19.trna8 | ACCTGGGTAGCTTAGTTGGTAGAGCATTGGACTttaA | 277 |
| | | ATTTGAGGGCCCAGGTTTCAAGTCCCTGTTTGGGTG | |
| 22 | LysTAAchr2.trna15 | GTTGGGGTAACTCAGTTGGTAGAGTAGCAGACTttaC | 278 |
| | | ATCTGAGGGtCCAGGGTTTAAGTCCATGTCCAGGCA | |
| 23 | LysTAAchr11.trna11 | GCCTGGATAGCTCAGTTGGTAGAGCATCAGACTttaA | 279 |
| | | ATCTGAGGGtCCAGGGTTCAAGTCCCTGTTCAGGCG | |
| 24 | LysTAAchr6.trna144 | GCCTGGATAGCTCAGTCGGTAGAGCATCAGACTttaA | 280 |
| | | ATCTGAGGGtCCAGGGTTCAAGTCCCTGTTCAGGCG | |
| 25 | LysTAAchr11.trna5 | GCCCGGATAGCTCAGTCGGTAGAGCATCAGACTttaA | 281 |
| | | ATCTGAGGGtCCGGGGTTCAAGTCCCTGTTCGGGCG | |
| 26 | LysTAAchr6.trna150 | GCCTGGGTAGCTCAGTCGGTAGAGCATCAGACTttaA | 282 |
| | | ATCTGAGGGtCCAGGGTTCAAGTCCCTGTCCAGGCG | |
| 27 | LysTAAchr6.trna70 | GCCTGGATAGCTCAGTTGGTAGAACATCAGACTttaA | 283 |
| | | ATCTGACGGtGCAGGGTTCAAGTCCCTGTTCAGGCG | |
| 29 | LysTAAchr6.trna53 | ACCTGGGTAGCTCAGTAGGTAGAACATCAGACTtta | 284 |
| | | ATCTGAGGGtCTAGGGTTCAAGTCCCTGTCCAGGCG | |
| 1 | LysTAGchr19.trna6 | GCCCAGCTAGCTCAGTCGGTAGAGCATAAGACTctaA | 285 |
| | | ATCTCAGGGtTGTGGATTCGTGCCCCATGCTGGGTG | |
| 8 | LysTAGchr16.trna31 | GCCCGGCTAGCTCAGTCGGTAGAGCATGAGACCctaA | 286 |
| | | ATCTCAGGGtCGTGGGTTCGAGCCCCACGTTGGGCG | |
| 9 | LysTAGchr16.trna11 | GCCCGGCTAGCTCAGTCGGTAGAGCATGGGACTctaA | 287 |
| | | ATCTCAGGGtCGTGGGTTCGAGCCCCACGTTGGGCG | |
| 16 | LysTAGchr16.trna23 | GCCTGGATAGCTCAGTTGGTAGAGCATCAGACTctaA | 288 |
| | | ATCTGAGGGtCCAGGGTTCAAGTCCCTGTTCAGGCA | |
| 19 | LysTAGchr19.trna8 | ACCTGGGTAGCTTAGTTGGTAGAGCATTGGACTctaA | 289 |
| | | ATTTGAGGGCCCAGGTTTCAAGTCCCTGTTTGGGTG | |
| 23 | LysTAGchr11.trna11 | GCCTGGATAGCTCAGTTGGTAGAGCATCAGACTctaA | 290 |
| | | ATCTGAGGGtCCAGGGTTCAAGTCCCTGTTCAGGCG | |
| 24 | LysTAGchr6.trna144 | GCCTGGATAGCTCAGTCGGTAGAGCATCAGACTctaA | 291 |
| | | ATCTGAGGGtCCAGGGTTCAAGTCCCTGTTCAGGCG | |
| 25 | LysTAGchr11.trna5 | GCCCGGATAGCTCAGTCGGTAGAGCATCAGACTctaA | 292 |
| | | ATCTGAGGGtCCGGGGTTCAAGTCCCTGTTCGGGCG | |
| 26 | LysTAGchr6.trna150 | GCCTGGGTAGCTCAGTCGGTAGAGCATCAGACTctaA | 293 |
| | | ATCTGAGGGtCCAGGGTTCAAGTCCCTGTCCAGGCG | |
| 27 | LysTAGchr6.trna70 | GCCTGGATAGCTCAGTTGGTAGAACATCAGACTctaA | 294 |
| | | ATCTGACGGtGCAGGGTTCAAGTCCCTGTTCAGGCG | |
| 4 | CysTGAchr7.trna8 | GGGGGCATAGCTCAGTGGTAGAGCATTTGACTtcaGA | 295 |
| | | TCAAGAGGtCCCTGGTTCAAATCCAGGTGCCCCCT | |
| 6 | CysTGAchr7.trna24 | GGGGGTATAGCTTAGCGGTAGAGCATTTGACTctaGA | 296 |
| | | TCAAGAGGtCCCCGGTTCAAATCCGGGTGCCCCCT | |
| 7 | CysTGAchr7.trna20 | GGGGGTATAGCTTAGGGGTAGAGCATTTGACTctaGA | 297 |
| | | TCAAAAGGtCCCTGGTTCAAATCCAGGTGCCCCTT | |
| 8 | CysTGAchr7.trna29 | GGGGGTATAGCTCAGGGGTAGAGCATTTGACTctaGA | 298 |
| | | TCAAGAGGtCCCCAGTTCAAATCTGGGTGCCCCCT | |
| 10 | CysTGAchr7.trna13 | GGGGGTATAGCTCAGGGGTAGAGCATTTGACTctaGA | 299 |
| | | TCAAGAGGtCTCTGGTTCAAATCCAGGTGCCCCCT | |
| 21 | CysTGAchr7.trna23 | GGGGGTATAGCTCAGGGGTAGAGCACTTGACTctaGA | 300 |
| | | TCAAGAGGtCCCTGGTTCAAATCCAGGTGCCCCCT | |
| 22 | CysTGAchr17.trna27 | GGGGGTATAGCTCAGTGGTAGAGCATTTGACTctaGA | 301 |
| | | TCAAGAGGtCCCTGGTTCAAATCCGGGTGCCCCCT | |
| 23 | CysTGAchr15.trna3 | GGGGGTATAGCTCAGTGGGTAGAGCATTTGACTctaG | 302 |
| | | ATCAAGAGGtCCCCGGTTCAAATCCGGGTGCCCCCT | |
| 24 | CysTGAchr3.trna6 | GGGGGTGTAGCTCAGTGGTAGAGCATTTGACTctaGA | 303 |
| | | TCAAGAGGtCCCTGGTTCAAATCCAGGTGCCCCCT | |
| 25 | CysTGAchr14.trna9 | GGGGGTATAGCTCAGGGGTAGAGCATTTGACTctaGA | 304 |
| | | TCAAGAGGtCCCCGGTTCAAATCCGGGTGCCCCCT | |
| 26 | CysTGAchr3.trna5 | GGGGGTATAGCTCAGGGGTAGAGCATTTGACTctaGA | 305 |
| | | TCAAGAGGtCCCTGGTTCAAATCCAGGTGCCCCCT | |

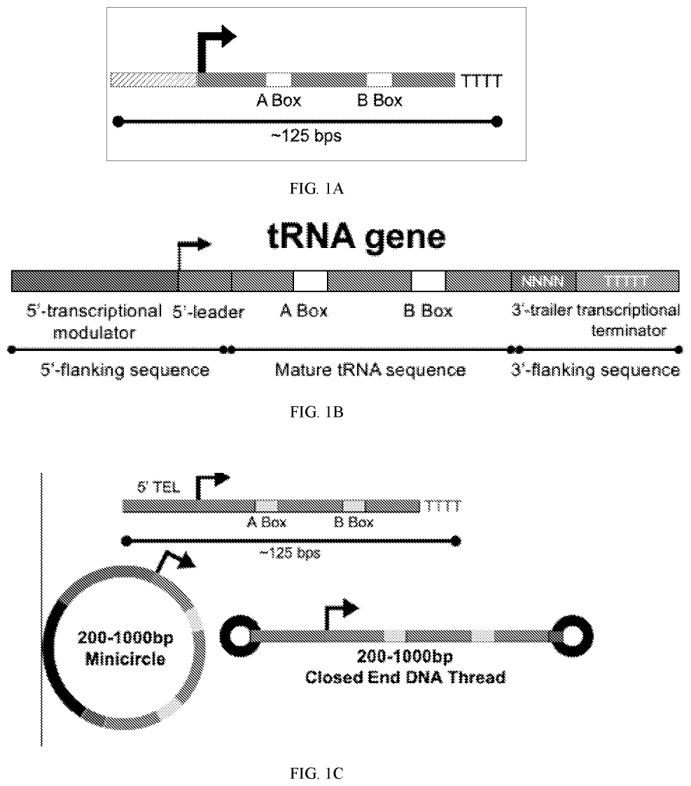

As disclosed herein, ACE-tRNAs gene structure is well-suited for PTC therapeutics. tRNA genes are transcribed into tRNAs by type 2 RNA polymerase (Pol) III recognition of internal promoter elements (A and B boxes, FIG. 1), wherein the tRNAs are flanked by a short (<50 bp) 5′ flanking region and a 3′ transcription termination elements consisting of a short run of thymidine nucleotides (˜4 Thymidines, Ts). Most tRNA genes are 72-76 bps in length, and therefore an entire tRNA expression cassette can consist of only about 125 bps (FIG. 1).

There are a variety of sequence elements involved with transcription and translational function of the ACE-tRNA that can be optimized. RNA polymerase III drives expression of tRNA genes in eukaryotes utilizing type 2 intragenic promoter elements (A and B boxes, FIG. 1A or 1B). While the A and B boxes are sufficient for type 2 promoter function, the expression of tRNAs may be modulated and enhanced by the sequence about 50 bp immediately 5′ to the gene (5′-flanking sequence). Transcription of tRNA is terminated by a short stretch of thymidine nucle-otides (≤4 thymidines, Ts).

As disclosed herein, the inventors have taken advantage of tRNAs' unique gene features in generating small DNA vectors, such as minicircle (MC) and Closed-End DNA Threads (CEDTs), for development of a therapeutic that exhibits efficient and persistent suppression of disease-causing PTCs, including those that reside within the CFTR gene that result in cystic fibrosis (“CF”).

This ACE-tRNA approach offers several significant benefits over other readthrough strategies, including (1) codon specificity; (2) ACE-tRNAs suppression of PTCs resulting in seamless rescue, thus negating spurious effects on protein stability, folding, trafficking and function; and (3) in vitro delivery of these of ACE-tRNA resulting in significant functional rescue of affected protein, such as CFTR channels with p.G542X or p.W1282X CF mutations. Preliminary results exhibited minimal suppression of endogenous translation termination codons, suggesting insignificant side-effects on the translatome. The ACE-tRNAs have shown to be efficient at PTC suppression in several cDNA genes with varied PTC positions in multiple cell-types. Because ACE-tRNAs exhibit high efficiency in PTC suppression with no known detrimental effects, they can be used as therapeutics.

### Minivectors

One aspect of this invention relates to generation and in vivo delivery of small DNA minivectors encoding suppression tRNAs for the purpose of PTC therapeutics. When considering optimal therapeutic attributes, the gold-standard is a one-time cure. However, outside of vaccines, these cures have rarely been realized. Recently, CRISPR/Cas9 has gained traction as a promising therapeutic because of its ability to make permanent changes to the genome. Importantly, genomic manipulations, including those made by CRISPR/Cas9, only last as long as the life span of modified cells, unless stem cell populations are targeted. With turnover of cells, redelivery of the therapeutic is necessary and immune responses to the therapeutic must be considered. Therefore, inventors set out to design a PTC therapeutic based on the ACE-tRNA platform with limited immunogenicity to allow repetitive delivery, a half-life that matches target cells (e.g., airway epithelium cells), reduced pathogenicity and low capacity for insertional mutagenesis.

In some embodiments, two small cDNA vector formats, Minicircles (MCs) and Closed-End DNA Threads (CEDTs), also known as DNA ministrings, Doggybone DNA™ or linear covalently closed (LCC) DNA, are generated and used. For example, small DNA vectors (minivectors) that express ACE-tRNAs are used to obtain in vivo data sets in cells, mice and pig to develop treatments of nonsense-associated disorders, including CF. In embodiments directed to DNA molecules having therapeutic utility, the DNA template will typically comprise an expression cassette comprising one or more promoter or enhancer elements and a gene or other coding sequence which encodes an RNA or protein of interest.

A vector of this invention typically comprises an expression cassette as described above, i.e., comprising, consisting or consisting essentially of a eukaryotic promoter operably linked to a sequence encoding a gene or protein of interest, and optionally a eukaryotic transcription termination sequence. Optionally the expression cassette may be a minimal expression cassette as defined below, i.e., lacking one or more bacterial or vector sequences, typically selected from the group consisting of: (i) bacterial origins of replication; (ii) bacterial selection markers (typically antibiotic resistance genes) and (iii) unmethylated CpG motifs.

Preferably, a vector of this invention does not contain any of such unnecessary sequences. That is, the vector is free of such unnecessary sequences. Such unnecessary or extraneous sequences (also described as bacterial or vector sequences) may include bacterial origins of replication, bacterial selection markers (e.g., antibiotic resistance genes), and unmethylated CpG dinucleotides. Deletion of such sequences creates a “minimal” expression cassette which does not contain extraneous genetic material. Also, bacterial sequences of the type described above can be problematic in some therapeutic approaches. For example, within a mammalian cell, bacterial/plasmid DNA can cause the cloned gene to switch off such that sustained expression of the gene or protein of interest cannot be achieved. Also, antibiotic resistance genes used in bacterial propagation can cause a risk to human health. Furthermore, bacterial plasmid/vector DNA may trigger an unwanted non-specific immune response. A specific characteristic of bacterial DNA sequences, the presence of unmethylated cytosine-guanine dinucleotides, typically known as CpG motifs, may also lead to undesired immune responses.

### Minicircle (MC)

MCs are small circular vectors (<5 kilobases (kb)) that are constructed to contain only the minimal sequences for gene expression, generally a promoter, gene of interest (GOI) and termination sequence. The small size increases cell-entry and intracellular trafficking to the nucleus, resulting in increased delivery and transcription bioavailability. Furthermore, they are devoid of bacterial sequences commonly found in plasmids which reduces pathogenicity and episomal silencing for prolonged expression of the encoded GOI. In liver it has been demonstrated that MCs express GOI for >115 days with little to no reduction in expression before study termination.

Airway epithelia have half-lives of 6 months in the trachea and 17 months in the bronchioles in mice and 50 days in humans. Small DNA vectors, mostly MCs >3 kb in size, can be delivered broadly in vivo in several ways including Polyethylenimine (PEI), Receptor-Targeted Polyplexes, liposomes, hydrodynamic injection and by electroporation. MCs encoding CpG-less CFTR (˜7 kb) have been delivered to mouse lung by PEI condensation and aerosolization which resulted in sustained expression 56 days following delivery.

### Closed-End DNA Thread (CEDT)

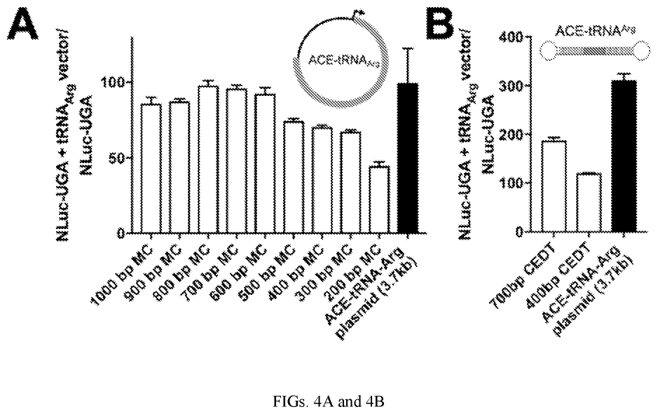

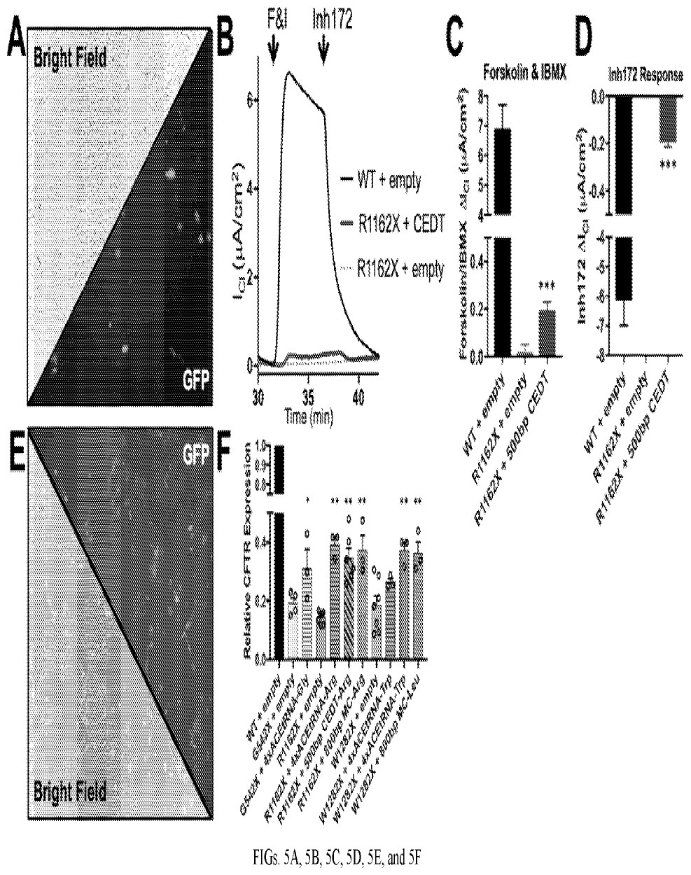

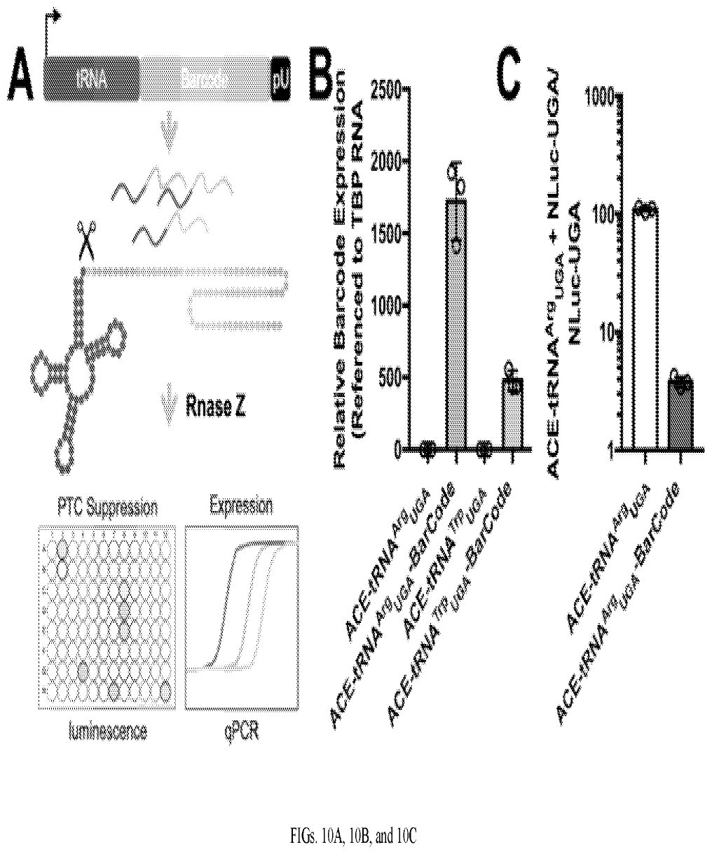

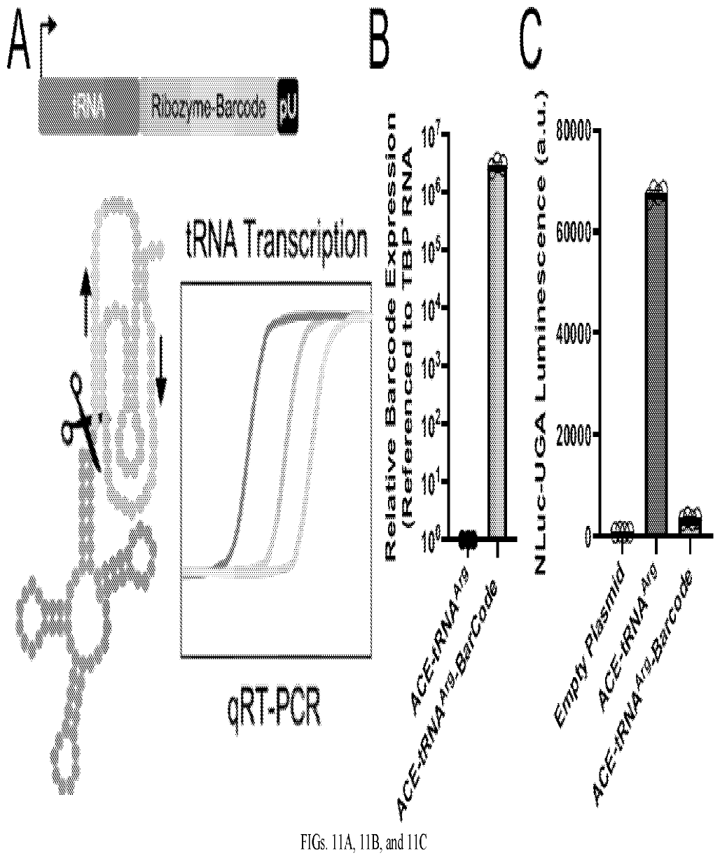

As used herein, a “Closed-End DNA Thread” or “CEDT” refers to a closed linear DNA molecule. Such a closed-end linear DNA molecule may be viewed as a single stranded circular molecule as depicted in FIG. 4B and FIG. 8D. A CEDT molecule typically comprises covalently closed ends also described as hairpin loops, where base-pairing between complementary DNA strands is not present. The hairpin loops join the ends of complementary DNA strands. Structures of this type typically form at the telomeric ends of chromosomes in order to protect against loss or damage of chromosomal DNA by sequestering the terminal nucleotides in a closed structure. In certain examples of CEDT molecules described herein, hairpin loops flank complementary base-paired DNA strands, forming a “doggy-bone” shaped structure (as shown in FIGS. 4B and 8D).

A CEDT molecule typically comprises a linear double stranded section of DNA with covalently closed ends, i.e., hairpin ends. The hairpins join the ends of the linear double DNA strands, such that if the molecule was completely denatured, a single stranded circular DNA molecule would be produced. Usually, a CEDT as described herein is essentially fully complementary in sequence, although some minor variations or “wobbles” may be tolerated by the structure. Thus, the closed linear DNA or CEDT may be at least 75%, 80%, 85%, 90%, or 95% complementary, or at least 96, 97, 98, 99 or 100% complementary in sequence. When denatured, it is effectively a circular molecule comprising both forward (sense or plus) and reverse (antisense or minus) strands adjacent to each other. This is in contrast to plasmid DNA or MC DNA where the complementary sequences (minus and plus) lie on separate circular strands (FIG. 4A compared to FIG. 4B).

The bases within the apex (end or turn) of the hairpin may not be able to form base pairs, due to the conformational stress put onto the DNA strand at this point. For example, at least 2 base pairs at the apex of the portion of the apex may not form base-pairs, but the exact conformation is likely to be subject to fluctuations depending on the conditions in which the DNA is maintained, and the exact sequences around the hairpin. Thus, 2 or more bases may not be able to form pairs given the structural distortion involved, despite their complementary nature. Some “wobbles” of non-complementary bases within the length of a hairpin may not affect the structure. A wobble may be a break in the palindrome, but the sequences may remain complementary. It is, however, preferred that the sequence of the hairpin is entirely self-complementary.

Complementarity describes how the bases of each polynucleotide in a sequence (5′ to 3′) are in a hydrogen-bonded pair with a complementary base, A to T (or U) and C to G on the anti-parallel (3′ to 5′) strand, which may be the same strand (internal complementary sequences) or on a different strand. This definition applies to any aspect or embodiment of the invention. It is preferred that the sequences in the hairpin are 90% complementary, preferably 91%, 92%, 93%, 94%, 95%, 96%, 98%, 99% or 100% complementary.

A CEDT may comprise any sequence within the double stranded sequence, either naturally derived or artificial. It may comprise at least one processing enzyme target sequence, such as one, two, three, four or more processing enzyme target sites. Such a target sequence is to allow for the DNA to be optionally processed further following synthesis. A processing enzyme is an enzyme that recognizes its target site and processes the DNA. The processing enzyme target sequence may be a target sequence for a restriction enzyme. A restriction enzyme, i.e., a restriction endonuclease, binds to a target sequence and cleaves at a specific point. The processing enzyme target sequence may be a target for a recombinase. A recombinase directionally catalyzes a DNA exchange reactions between short (30-40 nucleotides) target site sequences that are specific to each recombinase. Examples of recombinases include the Cre recombinase (with loxP as a target sequence) and FLP recombinase (with short flippase recognition target (FRT) sites). The processing enzyme target sequence may be a target for a site-specific integrase, such as the phiC31 integrase.

The processing enzyme target sequence may be a target sequence for a RNA polymerase too, such that the CEDT becomes a template for RNA synthesis. In this instance, the processing enzyme targeting site is a promoter, preferably a eukaryotic promoter. To that end, a CEDT may comprise an expression cassette comprising, consisting or consisting essentially of a eukaryotic promoter operably linked to a sequence enclosing a RNA (e.g., a tRNA) or protein of interest, and optionally a eukaryotic transcription termination sequence. A “promoter” is a nucleotide sequence which initiates and regulates transcription of a polynucleotide. “Operably linked” refers to an arrangement of elements wherein the components so described are configured so as to perform their usual function. Thus, a given promoter operably linked to a nucleic acid sequence is capable of effecting the expression of that sequence when the proper enzymes are present. The term “operably linked” is intended to encompass any spacing or orientation of the promoter element and the DNA sequence of interest which allows for initiation of transcription of the DNA sequence of interest upon recognition of the promoter element by a transcription complex.

The CEDT or MC may be of any suitable length. Particularly, the CEDT or MC may be up to 4 kb. Preferably the DNA template may be 100 bp to 2 kb, 200 bp to 1 kb, most preferably 200 bp to 800 bp. MCs and CEDTs that are 200 bp or longer can accommodate multiple ACE-tRNA cassette copies. This can allow for higher ACE-tRNA expression from each MC and CEDT unit. Having multiple copies of ACE-tRNAs from each minivector allows one to include one or more sequences into each unit. For instance, a Leucine ACE-tRNA and a Tryptophan ACE-tRNA can be include in one MC or CEDT minivector. Both of these ACE-tRNAs can be effective in cystic fibrosis for rescuing or suppressing the mutation W1282X-CFTR and significantly enhance suppression activity because they utilize different tRNA aminoacylsynthetases.

Closed DNA molecules have utility as therapeutic agents i.e., DNA medicines which can be used to express a gene product in vivo. This is because their covalently closed structure prevents attack by enzymes such as exonucleases, leading to enhanced stability and longevity of gene expression as compared to “open” DNA molecules with exposed DNA ends. Linear double stranded open-ended cassettes have been demonstrated to be inefficient with respect to gene expression when introduced into host tissue. This has been attributed to cassette instability due to the action of exonucleases in the extracellular space.

Sequestering DNA ends inside covalently closed structures also has other advantages. The DNA ends are prevented from integrating with genomic DNA and so closed linear DNA molecules are of improved safety. Also, the closed linear structure prevents concatamerisation of DNA molecules inside host cells and thus expression levels of the gene product can be regulated in a more sensitive manner.

A CEDT has all of the same benefits as MCs but exhibit a linear DNA cassette topology with covalently closed ends. The CEDT has been used in vivo for expression of antigens for generation of vaccines because of their ease of production, delivery efficiency, low pathogenicity and sustained expression. CEDTs have some advantages over MCs as they can be made completely synthetically with ease in high abundance at GMP grade, thus allowing rapid design and manufacturing.

CEDTs can be made using methods known in the art. A cell-free production of CEDTs has been described in U.S. Pat. No. 9,499,847, US20190185924, WO2010/086626, and WO2012/017210, which are hereby incorporated by reference. The method relates to the production of linear double stranded DNA covalently closed at each end (closed linear DNA) using a DNA template, wherein the DNA template comprises at least one protelomerase recognition sequence, and where the template is amplified using at least one DNA polymerase and processed using a protelomerase enzyme to yield closed linear DNA. The closed ends of the closed linear DNA each include a portion of a protelomerase recognition sequence. The use of a closed linear DNA as a template is described in the listed applications, and the use of such a template is advantageous, since it means that the minimum amount of reagents are wasted during production. A CEDT molecule produced by these methods is linear, double stranded, and covalently closed at each end by a portion of a protelomerase recognition sequence. This linear, double stranded DNA molecule can include one or more stem loop motifs.

As disclosed herein, in one example, this disclosure evaluates the delivery efficiency of ACE-tRNA expressing minivectors, such as MC and CEDT minivectors, to lung using electric fields and effectiveness and persistence of ACE-tRNAs suppression of PTCs in airway epithelial cells in vitro and in vivo. Taking advantage of the tiny size of ACE-tRNA genes (about 80 nt), inventors have successfully generated the smallest ever therapeutic expression vector to their knowledge.

With a library of effective ACE-tRNAs in hand, one can determine the most effective delivery method in vivo. As disclosed herein, one can encode ACE-tRNA^{Trp}_{UGA}, ACE-tRNA^{Leu}_{UGA}, ACE-tRNA^{Gly}_{UGA} and ACE-tRNA^{Arg}_{UGA} in MCs and CEDTs to target the three most common CF nonsense mutations (p.G542X, p.W1282X and p.R553X). The MC and CEDT technologies, as deliverable platforms, can be paired with a multitude of delivery methods including nanoparticles, protein complexes, and electric fields, demonstrated here. MC and CEDT minivectors have several features that make them attractive for gene therapy including: (1) dramatically reduced size allowing them to overcome obstacles during intracellular trafficking, therefore improving bioavailability; (2) higher cell-entry efficiencies; (3) possession of transcriptionally active structure; and (4) sustained transgene expression without genomic integration.

### Design Structure

The design of minivector sequence has important implications on expression efficiency and persistence. As disclosed herein, the minivector, either MC or CEDT of this invention, in addition to the tRNA expression cassette as described herein, optionally includes a number of advantageous elements and/or fractures. Examples include ACE-tRNA-Barcoding Sequences (ABS), Transcription Enhancing 5′ Leader Sequences (TELS), and DNA nuclear Targeting Sequences (DTS).

One important feature is the CpG sequence content and the methylation thereof. DNA methylation and its impact on transcription has been extensively studied. Lack of methylation is a prerequisite for active transcription, where methylated CpG islands are found on the inactive X chromosome and on silenced alleles of parentally imprinted genes. Furthermore, it has been shown that in vitro methylation of DNA inhibits gene expression. Because the ACE-tRNA gene is exceedingly small and contains only four CpG sequences on average, inventors have been able to design their minivectors to be almost completely void of CpG sequences to enhance persistence of ACE-tRNA expression. Furthermore, even at 500 bps, the ACE-tRNA gene consumes only a fraction of the total payload leaving options for including 3′ ABS, TELS, and DTS. The implementation of ABS, TELS and DTSs are described in Examples 6 and 7 below.

The ability to suppress PTCs efficiently is governed significantly by the genetic landscape in which the PTC lies. Therefore, it is imperative to determine the efficiency of ACE-tRNAs expressed from minivectors of to suppress PTCs in target cells, such as those in human airway epithelia in culture. To that end, one can use TELS and DTS to enhance ACE-tRNA expression in vivo by increasing transcription activity and targeting minivectors to the nucleus.

TELS

Increasing expression of ACE-tRNAs from minivectors can decrease the burden of delivery efficiency on the therapeutic efficacy of ACE-tRNAs. Although it is well established that the transcription of eukaryotic tRNA genes is directed by intragenic promoter elements (A and B boxes), the 5′ flanking sequence region also significantly regulates tRNA transcription, presumably through interactions with the polIII transcription complex. Such region can be used as transcription enhancing 5′ leader sequence (TELS). In several instances, tRNA genes with an identical coding sequence, have different 5′-flanking sequences that increase or decrease transcription. Furthermore, 5′ flanking sequences are assumed to modulate tissue expression specificity. To that end, one can use a human tRNA Tyr 5′ leader sequence (5′-AGCGCTCCGGTTTTTCTGTGCTGAACCTCAGGGGACGCCGACACACGTACACGTC-3′, SEQ ID NO: 306) as TELS.

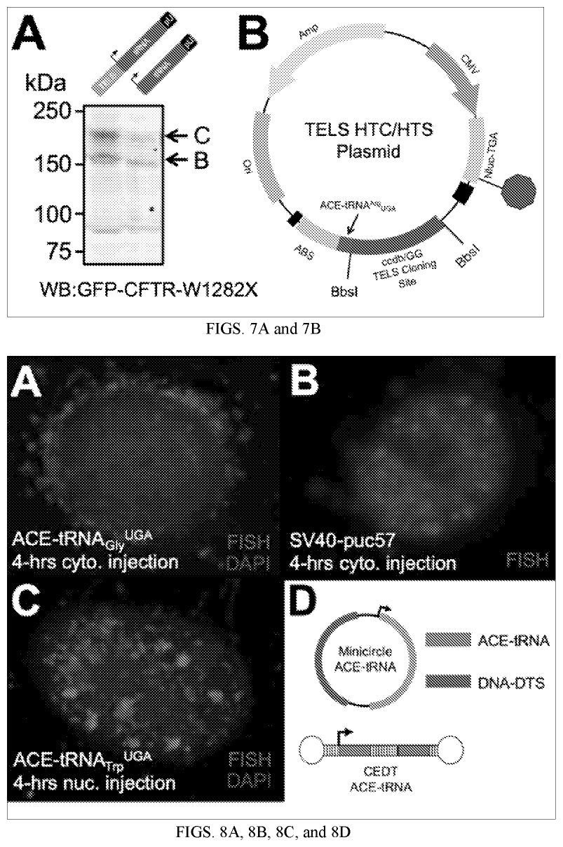

FIG. 7A shows that inclusion of the 5′ leader sequence increases the suppression activity of ACE-tRNA^{Trp}_{UGA} on W1282X-CFTR by more than 5 folds. About 416 tRNA genes have been annotated in the human genome (tRNAscan-SE database, Lowe et al., Nucleic Acids Res 25, 955-964, (1997)). The tRNA 5′ flanking sequences (1 kb) of all 416 tRNA genes can be used as TELS for this invention.

DTS

A DNA nuclear targeting sequence or DTS is a DNA sequence or repeats of a DNA sequence needed to support nuclear import of an otherwise cytoplasmically localized DNA. Naturally occurring DNA sequences in the promoters of viruses or in the promoters of mammalian genes provide nuclear entry of DNA containing a transgene by incorporating them into expression vectors that can be expressed in a non-dividing cell. A non-limiting example pf DTS is the DNA sequence from the SV40 genome, which contain the enhancer repeats (5′-atgctttgca tacttctgcc tgctggggag cctggggact ttccacaccc taactgacac acattccaca gctggttggt acctgca-3′, SEQ ID NO: 307). This SV40 DTS has been shown to support sequence-specific DNA nuclear import of plasmid DNA (e.g., Dean et al., Exp. Cell Res. 253:713-722, 1999). As disclosed herein, additional DTSs can be obtained in the manner described in the examples below.

Bar Codes

The minivector described in this invention can include one or more additional elements that allow direct measurement of ACE-tRNA transcription activity. One example is a barcode sequence.

Standard RNA sequencing (RNA-seq) methods have been used to analyze, mRNA, non-coding RNA, microRNA, small nuclear/nucleolar RNA and rRNA¹⁶⁵. However, tRNA is the only class of small cellular RNA where these standard RNA-seq methods cannot be implemented. Significant obstacles that encumber straightforward methods include the presence of post-transcriptional modifications that interfere with polymerase amplification and stable and extensive secondary structure that block adaptor ligation. Recently several methodologies have been developed to circumvent these issues, however these approaches are relatively involved and cost prohibitive. Furthermore, as the ACE-tRNA sequences differ by only one nucleotide from one or more endogenous tRNAs, norther blot, microarray and fragmented RNA-seq cannot discern expression level of exogenous therapeutic ACE-tRNAs and endogenous tRNAs. Several recent studies have utilized tRNAs as promoters to drive high expression levels of tRNA:sgRNA (Cas9 single guide RNA) fusion transcripts that are then efficiently and precisely cleaved by endogenous tRNase Z.

The minivector of the invention can include one or more ACE-tRNA-Barcoding Sequences or ABSs. An ABS can be at the 3′ or 5′ of the sequence encoding a ACE-tRNA so that the vector encodes a fusion transcript of the ACE-tRNA and the barcode. In one example, the barcode sequence is at the 3′ end and the minivector encode ACE-tRNA:barcode fusion transcripts, where the barcode sequence can be cleaved by endogenous tRNase Z, allowing for qPCR quantification to determine relative ACE-tRNA expression, while giving a fully functional ACE-tRNA (FIG. 10A). As shown in Example 11 below, the inventors designed a random 200 bp barcode sequence with no homology to mouse genomic sequence. When transfected into 16HBE14o- cells that stably express NLuc-UGA, ACE-tRNA^{Arg}_{UGA}- and ACE-tRNA^{Trp}_{UGA}-Barcode exhibit robust expression as monitored by qPCR, while the non-barcoded ACE-tRNA gave no significant signal (FIG. 10B). Although in one case, the suppression activity of the ACE-tRNA^{Arg}_{UGA} may be impeded by the presence of a 3′ barcode sequence, the deficit in ACE-tRNA activity most likely due to poor 3′ processing, which can be determined by northern blot probed towards the barcode sequence. Such deficit can be corrected by using a 3′ hepatitis delta virus (HDV) self-cleaving ribozymes whose sequence can also act as a barcode or modified barcode linker sequence to improve 3′ processing. Incorporation of 3′ ribozymes can result in improved ACE-tRNA 3′ processing and increase their PTC suppression activity. Furthermore, the ABS technology allows measurements of ACE-tRNA expression to compliment the NLuc PTC reporter (FIG. 9).

In any embodiment of the present application, the nucleic acid molecule or vector described herein can optionally comprise one or more reporter molecule. A reporter is a molecule whose expression in a cell confers a detectable trait to the cell. In various embodiments, reporters include, but are not limited to, chloramphenicol-acetyl transferase (CAT), β-galactosyltransferase, horseradish peroxidase, luciferase, NanoLuc®, alkaline phosphatase, and fluorescent proteins including, but not limited to, green fluorescent proteins (e.g., GFP, TagGFP, T-Sapphire, Azami Green, Emerald, mWasabi, mClover3), red fluorescent proteins (e.g., tdTomato, mRFP1, JRed, HcRed1, AsRed2, AQ143, mCherry, mRuby3, mPlum), yellow fluorescent proteins (e.g., EYFP, mBanana, mCitrine, PhiYFP, TagYFP, Topaz, Venus), orange fluorescent proteins (e.g., DsRed, Tomato, Kusabria Orange, mOrange, mTangerine, TagRFP), cyan fluorescent proteins (e.g., CFP, mTFP1, Cerulean, CyPet, AmCyanl), blue fluorescent proteins (e.g., Azurite, mtagBFP2, EBFP, EBFP2, Y66H), near-infrared fluorescent proteins (e.g., iRFP670, iRFP682, iRFP702, iRFP713 and iRFP720), infrared fluorescent proteins (e.g., IFP1.4) and photoactivatable fluorescent proteins (e.g., Kaede, Eos, IrisFP, PS-CFP).

### Introduction of Nucleic Acid Encoding ACE-tRNAs to Cells

Exogenous genetic material (e.g., a nucleic acid or a minivector encoding one or more therapeutic ACE-tRNAs) can be introduced into a target cells of interest in vivo by genetic transfer methods, such as transfection or transduction, to provide a genetically modified cell. Various expression vectors (i.e., vehicles for facilitating delivery of exogenous genetic material into a target cell) are known to one of ordinary skill in the art. As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into a tRNA.

As used herein, “transfection of cells” refers to the acquisition by a cell of new genetic material by incorporation of added nucleic acid (DNA, RNA, or a hybrid thereof). Thus, transfection refers to the introducing of nucleic acid into a cell using physical or chemical methods. Several transfection techniques are known to those of ordinary skill in the art including: calcium phosphate nucleic acid co-precipitation, strontium phosphate nucleic acid co-precipitation, DEAE-dextran, electroporation, cationic liposome-mediated transfection, and tungsten particle-facilitated microparticle bombardment. In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes a heterologous gene (coding for a therapeutic RNA or protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion, an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, ubiquitin, elongation factor-1 and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene, and/or signal sequence is deemed to be within the scope of one of ordinary skill in the art without undue experimentation.

An ACE-tRNA construct of the present invention can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Addition of DNA binding proteins such as Transcription Factor A Mitochondria (TFAM) can be used to condense DNA and shield charge. Due to the small size and compact shape, the DNA:Protein (DNP) complexes can then be delivered to cells by cell penetrating peptides, PEG derivative, liposomes or electroporation. In some instances, DNA binding proteins can encode nuclear localization signals to actively transport of DNPs from the cytoplasm to the nucleus where the DNA minivectors are transcribed.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The nucleic acid molecule of the invention can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the composition described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the composition into tissue without the use of a needle. The MID may inject the composition as a small stream or jet with such force that the composition pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference. The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired composition in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the composition into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver compositions to all types of tissues, particularly to skin and mucosa. In some embodiments, a needle-free injector may be used to propel a liquid that contains the composition to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle composition injectors that deliver the composition and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the ELGEN 1000 system, described in U.S. Pat. No. 7,328,064, the contents of which are herein incorporated by reference.

The MID may be a CELLECTRA (INOVIO Pharmaceuticals) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference. The MID may be an ELGEN 1000 system (INOVIO Pharmaceuticals). The ELGEN 1000 system may comprise device that provides a hollow needle; and fluid delivery means, wherein the apparatus is adapted to actuate the fluid delivery means in use so as to concurrently (for example automatically) inject fluid, the described composition herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue, such as tumor tissue, skin, tissue, liver tissue, and muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays known to those of skill in the art.

In one embodiment, this invention describes the effectiveness of ACE-tRNA encoding MCs and CEDTs to rescue CFTR mRNA expression and channel function in human nonsense CF cell culture models. For example, following delivery of ACE-tRNA encoding MCs and CEDTs to p.G542X, p.R1162X, p.W1282X 16HBE14oe- cells, rescue of CFTR mRNA expression from NMD is determined by qPCR and CFTR function assessed by Ussing chamber recordings. Also disclosed is to enhance the minivector therapeutic capabilities via a number of ways, including (1) using a library of Transcription Enhancing 5′ Leader Sequences (TELS) to increase ACE-tRNA expression and (2) using a library of DNA Targeting Sequences (DTSs) to increase nuclear targeting and transcriptional bioavailability.

Accordingly, the DNA minivector (either MC or CEDT) of the present invention comprises a promoter and a nucleic acid sequence encoding an anti-codon edited-tRNA. The DNA minivector further comprises one or two or three or four DNA sequences selected from the group consisting of a TELS, a DTS, a ABS and a reporter nucleic acid sequence.

In one embodiment, the DNA minivector comprises a promoter, a sequence encoding an anti-codon edited-tRNA, and a TELS.

In one embodiment, the DNA minivector comprises a promoter, a sequence encoding an anti-codon edited-tRNA, a TELS and a DTS.

In one embodiment, the DNA minivector comprises a promoter, a sequence encoding an anti-codon edited-tRNA, a TELS, a DTS and an ABS.

In one embodiment, the DNA minivector comprises a promoter, a sequence encoding an anti-codon edited-tRNA, a TELS, a DTS, an ABS and a reporter sequence.

In any of the DNA minivectors described herein, the DNA minivector is MC.

In any of the DNA minivectors described herein, the DNA minivector is CEDT.

In another embodiment, this invention describes the effectiveness and persistence of nonsense suppression by ACE-tRNA encoding plasmids, MCs and CEDTS in airway epithelia of p.G542X-CFTR and p.W1282X-CFTR mice. ACE-tRNA expressing cDNA plasmids, MCs and CEDTs can be delivered to wild type, p.G542X-CFTR and p.W1282X-CFTR mouse lungs using electric fields. Steady state CFTR mRNA expression can be measured by qPCR and CFTR protein by immunofluorescence (IF) and Western blot (WB). Cell-specific delivery of vectors can be determined by fluorescence in situ hybridization (FISH) in dissected lung segments. Endpoints can be assessed out to 42 days following a single delivery. Persistence of ACE-tRNA expression from vectors can be quantified in wild type mouse lung by subsequent delivery of a PTC reporter vector at 7 days, 14 days, and 1, 2, 6, and 12 months and qPCR of an ACE-tRNA Barcode Sequence.

In yet another embodiment, this invention describes the efficiency and persistence of ACE-tRNAs encoded in MCs and CEDTs following delivery into lungs of other larger lab animals. Wild-type animals can receive MC and CEDTs by electric field pulse. Persistence of ACE-tRNA expression and lung mapping of delivery can be determined out to 6 months by subsequent delivery of a PTC reporter vector and qPCR of an ACE-tRNA Barcode Sequence paired with Fluorescence In Situ Hybridization (FISH) and Immunofluorescence (IF).

### Disease Conditions and Methods of Treatment

Certain embodiments of the present disclosure provide a method of treating a disease or disorder associated with PTCs in a mammal (such as a human) comprising administering a vector encoding a therapeutic agent (e.g., an ACE-tRNA) as described herein to the mammal. Certain embodiments of the present disclosure provide a use of a therapeutic agent or vector encoding a therapeutic agent as described herein to prepare a medicament useful for treating the disease in a mammal.

Diseases or disorders associated with PTCs include, but are not limited to, variants of Duchenne muscular dystrophies and Becker muscular dystrophies due to a PTC in dystrophin, retinoblastoma due to a PTC in RBI, neurofibromatosis due to a PTC in NF1 or NF2, ataxia-telangiectasia due to a PTC in ATM, Tay-Sachs disease due to a PTC in HEXA, cystic fibrosis due to a PTC in CFTR, Wilm's tumor due to a PTC in WT1, hemophilia A due to a PTC in factor VIII, hemophilia B due to a PTC in factor IX, p53-associated cancers due to a PTC in p53, Menkes disease, Ullrich's disease, β-Thalassemia due to a PTC in betaglobin, type 2A and type 3 von Willebrand disease due to a PTC in Willebrand factor, Robinow syndrome, brachydactyly type B (shortening of digits and metacarpals), inherited susceptibility to mycobacterial infection due to a PTC in IFNGR1, inherited retinal disease due to a PTC in CRX, inherited bleeding tendency due to a PTC in Coagulation factor X, inherited blindness due to a PTC in Rhodopsin, congenital neurosensory deafness and colonic agangliosis due to a PTC in SOX10 and inherited neural develop-mental defect including neurosensory deafness, colonic agangliosis, peripheral neuropathy and central dysmyelinating leukodystrophy due to a PTC in SOX 10, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and ricketsand many others. The present invention in one embodiment includes compositions and methods for treating cystic fibrosis by reversing the effects of mutations present that are associated with nonsense mutations through introduction of the ACE-tRNAs of the invention. Additional disorders include Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, and Polycystic kidney disease.

Diseases or disorders associated with PTCs that can be treated by the DNA molecules and method described herein also include a number of eye diseases. Examples of the diseases and genes with the specific mutations include:

Cone dystrophies (Stargardt's disease (STGD1), cone-rod dystrophy, retinitis pigmentosa (RP), and increased susceptibility to age-related macular degeneration): KCNV2 Glu143X; KCNV2 Glu306X; KCNV2 Gln76X; KCNV2 Glu148X; CACNA2D4, Tyr802X; CACNA2D4, Arg628X; RP2, Arg120X; Rho, Ser334X; Rpe65, Arg44X; PDE6A, Lys455X;

Congenital stationary night blindness 2 (CSNB2): CACNA1F, Arg958X; CACNA1F, Arg830X

Congenital stationary night blindness 1 (CSNB1): TRPM1, Gln11X; TRPM1, Lys294X; TRPM1, Arg977X; TRPM1, Ser882X; NYX, W350X

Best Disease or BVMD, BEST1, Tyr29X; BEST1, Arg200X; BEST1, Ser517X

Leber congenital amaurosis (LCA): KCNJ13, Trp53X; KCNJ13, Arg166X; CEP290, Arg151X; CEP290, Gly1890X; CEP290, Lys1575X; CEP290, Arg1271X; CEP290, Arg1782X; CRB1, Cys1332X; GUCY2D, Ser448X; GUCY2D, Arg41091X; LCA5, G1n279X; RDH12, Tyr194X; RDH12, Glu275X; SPATA7, Arg108X; TULP1, Gln301X;

Usher syndrome 1: USH1C, Arg31X; PCDH15, Arg3X; PCDH15, Arg245X; PCDH15, Arg643X; PCDH15, Arg929X; IQCB1, Arg461X; IQCB1, Arg489X; PDE6A, Gln69X; ALMS1, Ser999X; ALMS1, Arg3804X;

Aniridia: Pax6, Gly194X

Ocular coloboma: Pax2, Arg139X; Lamb1, Arg524X

Choroideremia: REP1, Gln32X

According to one aspect, a cell expression system for expressing a therapeutic agent in a mammalian recipient is provided. The expression system (also referred to herein as a “genetically modified cell”) comprises a cell and an expression vector for expressing the therapeutic agent. Expression vectors include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a CEDT or MC minivector. Other examples of the expression vector include a recombinant adenoviral, adeno-associated virus, or lentivirus or retrovirus vector.

The expression vector further includes a promoter for controlling transcription of the heterologous gene. The promoter may be an inducible promoter. The expression system is suitable for administration to the mammalian recipient. The expression system may comprise a plurality of non-immortalized genetically modified cells, each cell containing at least one recombinant gene encoding at least one therapeutic agent.

The cell expression system can be formed in vivo. According to yet another aspect, a method for treating a mammalian recipient in vivo is provided. The method includes introducing an expression vector for expressing a heterologous gene product into a cell of the patient in situ, such as via intravenous administration. To form the expression system in vivo, an expression vector for expressing the therapeutic agent is introduced in vivo into the mammalian recipient i.v.

According to yet another aspect, a method for treating a mammalian recipient in vivo is provided. The method includes introducing the target therapeutic agent into the patient in vivo. The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

The present disclosure provides methods of treating a disease in a subject (e.g., a mammal) by administering an expression vector encoding ACE-tRNA to a cell or patient. For the gene therapy methods, a person having ordinary skill in the art of molecular biology and gene therapy would be able to determine, without undue experimentation, the appropriate dosages and routes of administration of the expression vector used in the novel methods of the present disclosure.

In certain embodiments, the agents and methods described herein can be used for the treatment/management of diseases that are caused by PTCs. Examples include, but are not limited to, Duchenne and Becker muscular dystrophies, retinoblastoma, neurofibromatosis, ataxia-telangiectasia, Tay-Sachs disease, cystic fibrosis, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. This therapy is advantageous in that it provides improved stop codon suppression specificity. The therapeutic ACE-tRNAs of the present invention target a specific stop-codon, TGA for instance, thus reducing off-target effects at stop-codons unrelated to disease. The present therapy is also advantageous in that it provides amino-acid specificity. The expressed tRNA is engineered to specifically replace the amino acid that was lost via insertion of the disease stop codon, thus negating any spurious effects on protein stability, folding and trafficking.

In certain embodiments, the present system is modular, and thus can be “personalized” to every possible disease PTC. For instance, there are nine individual tryptophan tRNAs in the human genome that are recognized by the Trp synthetase, all of which suppress the mRNA UGG codon. Thus, each of these nine Trp tRNA provides an opportunity for codon re-editing tolerance (UGG→UGA). Additionally, given their proximity to stop codons in the genetic code, the mutation of arginine codons to PTC nonsense codons are common in disease. There are over thirty Arg tRNAs that could be tested for codon editing tolerance and suppression efficacy. An ACE-tRNA that encodes and Arginine is a viable therapeutic for all Arg→PTC mutations regardless of gene. Indeed, 35% of LCA is caused by nonsense mutations and the majority of those are Arginine to stops. A further advantage of the present invention is that it provides facile expression and cell specific delivery, because the entire system (tRNA+promoter sequence) is compact.

### Formulations

Once the vector or another form of DNA produced in accordance with the invention has been generated and purified in a sufficient quantity, a process of the invention may further comprise its formulation as a DNA composition, for example a therapeutic DNA composition. A therapeutic DNA composition comprises a therapeutic DNA molecule of the type referred to above. Such a composition can comprise a therapeutically effective amount of the DNA in a form suitable for administration by a desired route e.g., an aerosol, an injectable composition or a formulation suitable for oral, mucosal or topical administration.

Formulation of DNA as a conventional pharmaceutical preparation may be done using standard pharmaceutical formulation chemistries and methodologies, which are available to those skilled in the art. Any pharmaceutically acceptable carrier or excipient may be used. Auxiliary substances, such as wetting or emulsifying agents, pH buffering substances and the like, may be present in the excipient or vehicle. These excipients, vehicles and auxiliary substances are generally pharmaceutical agents which may be administered without undue toxicity and which, in the case of vaccine compositions will not induce an immune response in the individual receiving the composition. A suitable carrier may be a liposome.

Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

The agents (e.g., a minivector) of the invention can be administered so as to result in a reduction in at least one symptom associated with a genetic disease (e.g., cystic fibrosis). The amount administered varies depending on various factors including, but not limited to, the composition chosen, the particular disease, the weight, the physical condition, and the age of the subject, and whether prevention or treatment is to be achieved. Such factors can be readily determined by the clinician employing animal models or other test systems that are well known to the art.

The present invention envisions treating a disease or disorder associated with a PTC by the administration of an agent, e.g., ACE-tRNA or an expression vector disclosed in this invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation), can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A pharmaceutically acceptable carrier can be a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well-known and readily available ingredients. The therapeutic agents of the invention can also be formulated as solutions appropriate for parenteral administration, for instance by intramuscular, subcutaneous or intravenous routes. The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water

### Nanoparticle Compositions

In some embodiments, the pharmaceutical compositions disclosed herein are formulated as lipid nanoparticles (LNP), such as those described in WO2020263883, WO2013123523, WO2012170930, WO2011127255 and WO2008103276; and US20130171646, each of which is herein incorporated by reference in its entirety. Accordingly, the present disclosure provides nanoparticle compositions comprising (i) a lipid composition comprising a delivery agent, and (ii) at least one nucleic acid, such as an ACE-tRNA or a DNA encoding the ACE-tRNA, e.g., a MC or CEDT. In such a nanoparticle composition, the lipid composition disclosed herein can encapsulate the nucleic acid.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles, liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and nucleic acid of interest. In some embodiments, the LNP comprises an ionizable lipid, a PEG-modified lipid, a sterol and a structural lipid. In some embodiments, the LNP has a molar ratio of about 20-60% ionizable lipid:about 5-25% structural lipid:about 25-55% sterol; and about 0.5-15% PEG- modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid.” In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively charged moieties include amine groups (e.g., primary, secondary, and/or tertiary amines), ammonium groups, pyridinium group, guanidine groups, and imidizolium groups. In a particular embodiment, the charged moieties can comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

IN some embodiments, the ionizable lipid is an ionizable amino lipid, sometimes referred to in the art as an “ionizable cationic lipid,” In one embodiment, the ionizable amino lipid may have a positively charged hydrophilic head and a hydrophobic tail that are connected via a linker structure. In addition to these, an ionizable lipid may also be a lipid including a cyclic amine group. In one embodiment, the ionizable lipid may be selected from, but not limited to, an ionizable lipid described in WO2013086354 and WO2013116126; the contents of each of which are herein incorporated by reference in their entirety. In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in WO2012170889, which is incorporated by reference in its entirety. In one embodiment, the lipid may be synthesized by methods known in the art and/or as described in WO2013086354, the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential. The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle.

In one embodiment, the nucleic acid described herein can formulated in lipid nanoparticles having a diameter from about 10 to about 100 nm. In one embodiment, the nanoparticles have a diameter from about 10 to 500 nm. In one embodiment, the nanoparticle has a diameter greater than 100 nm. In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The term “encapsulation efficiency” of a nucleic acid/polynucleotide describes the amount of the nucleic acid/polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement. Encapsulation efficiency is desirably high (e.g., close to 100%). The encapsulation efficiency can be measured, for example, by comparing the amount of the nucleic acid/polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a nucleic acid/polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a nucleic acid/polynucleotide present in a pharmaceutical composition disclosed herein can depend on multiple factors such as the size of the nucleic acid/polynucleotide, desired target and/or application, or other properties of the nanoparticle composition as well as on the properties of the nucleic acid/polynucleotide. For example, the amount of a nucleic acid/polynucleotide useful in a nanoparticle composition can depend on the size (expressed as length, or molecular mass), sequence, and other characteristics of the nucleic acid/polynucleotide. The relative amounts of a nucleic acid/polynucleotide in a nanoparticle composition can also vary. The relative amounts of the lipid composition and the nucleic acid/polynucleotide present in a lipid nanoparticle composition of the present disclosure can be optimized according to considerations of efficacy and tolerability.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015)“Delivery of oligonucleotides with lipid nanoparticles” Adv. Drug Deliv. Rev. 87:68-80; Silva et al. (2015)“Delivery Systems for Biopharmaceuticals. Part I: Nanoparticles and Microparticles” Curr. Pharm. Technol. 16: 940-954; Naseri et al. (2015)“Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC₃-DMA (MC₃), DLin-DMA, DLenDMA, DLin-D-DMA, DLin-K-DMA, DLin-M-C2-DMA, DLin-K-DMA, DLin-KC2-DMA, DLin-KC₃-DMA, DLin-KC4-DMA, DLin-C2K-DMA, DLin-MP-DMA, DODMA, 98N12-5, C₁2-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)-N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC₁4 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 Daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., WO2013033438 or US20130196948. The LNP formulation can also contain a polymer conjugate (e.g., a water-soluble conjugate) as described in, e.g., US20130059360, US20130196948, and US20130072709. Each of the references is herein incorporated by reference in its entirety.

The LNP formulations can comprise a conjugate to enhance the delivery of nanoparticles in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013339, 971-975, herein incorporated by reference in its entirety). As shown by Rodriguez et al. the self-peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., WO2012109121, which is incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in US20130183244, which is incorporated by reference in its entirety.

The LNP formulations can be engineered to alter the surface properties of particles so that the lipid nanoparticles can penetrate the mucosal barrier as described in U.S. Pat. No. 8,241,670 or WO2013110028, each of which is herein incorporated by reference in its entirety. The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin b4 dornase alfa, neltenexine, erdosteine) and various DNases including rhDNase. In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., WO2013110028, which is incorporated by reference in its entirety.

In some embodiments, the nucleic acids described herein can be formulated for controlled release and/or targeted delivery. As used herein, “controlled release” refers to a pharmaceutical composition or compound release profile that conforms to a particular pattern of release to effect a therapeutic outcome. In one embodiment, the nucleic acids can be encapsulated into a delivery agent described herein and/or known in the art for controlled release and/or targeted delivery. As used herein, the term “encapsulate” means to enclose, surround or encase. As it relates to the formulation of the nucleic acids of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 4050 or less of the pharmaceutical composition or compound of the invention can be enclosed, surrounded or encased within the delivery agent.

In some embodiments, the nucleic acid composition can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle composition described herein can be formulated as disclosed in WO2010075072, US20100216804, US20110217377, US20120201859 and US20130150295, each of which is herein incorporated by reference in their entirety. In some embodiments, the nanoparticle composition can be formulated to be target specific, such as those described in WO2008121949, WO2010005726, WO2010005725, WO2011084521 WO2011084518, US20100069426, US20120004293 and US20100104655, each of which is herein incorporated by reference in its entirety.

### Administration

The above-described therapeutic agents and compositions can be used for treating, protecting against, and/or preventing a PTC associated disease in a subject in need thereof by administering one or more composition described herein to the subject.

Such agents and compositions can be administered in dosages and by techniques well known to those skilled in the medical arts taking into consideration such factors as the age, sex, weight, and condition of the particular subject, and the route of administration. The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The agent or composition can be administered prophylactically or therapeutically. In therapeutic applications, the agents or compositions are administered to a subject in need thereof in an amount sufficient to elicit a therapeutic effect. An amount adequate to accomplish this is defined as “therapeutically effective dose.” Amounts effective for this use will depend on, e.g., the particular composition of the composition regimen administered, the manner of administration, the stage and severity of the disease, the general state of health of the subject, and the judgment of the prescribing physician.

The agent or composition can be administered by methods well known in the art as described in Donnelly et al. (Ann. Rev. Immunol. 15:617-648 (1997)), U.S. Pat. Nos. 5,580,859, 5,703,055, and 5,679,647, the contents of all of which are incorporated herein by reference in their entirety. The DNA of the composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector. The composition can be delivered via a variety of routes. Typical delivery routes include parenteral administration, e.g., intradermal, intramuscular or subcutaneous delivery. Other routes include oral administration, intranasal, and intravaginal routes. For the DNA of the composition in particular, the composition can be delivered to the interstitial spaces of tissues of an individual (U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (U.S. Pat. No. 5,679,647).

In one embodiment, the composition can be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the composition.

The composition can be a liquid preparation such as a suspension, syrup or elixir. The composition can also be a preparation for parenteral, subcutaneous, intradermal, intramuscular or intravenous administration (e.g., injectable administration), such as a sterile suspension or emulsion.

The composition can be incorporated into liposomes, microspheres or other polymer matrices (U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The ACE-tRNA or nucleic acid molecule encoding the ACE-tRNA may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

The ACE-tRNA or nucleic acid molecule encoding the ACE-tRNA may be delivered to the mammal by several well-known technologies including DNA injection with and without in vivo electroporation, liposome mediated, nanoparticle facilitated, recombinant vectors such as recombinant adenovirus, recombinant adenovirus associated virus and recombinant vaccinia. The ACE-tRNA or nucleic acid molecule encoding the ACE-tRNA may be delivered via DNA injection and along with in vivo electroporation.

### Electroporation

Administration of the composition via electroporation may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system or ELGEN electroporator to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50μβ, 20μβ, 10 or 1μβ, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the compositions of the present invention, include those described in U.S. Pat. No. 7,245,963 and US2005/0052630, the contents of which are hereby incorporated by reference in their entirety. Other electroporation devices and electroporation methods known in the art can also be used for facilitating delivery of the compositions. See, e.g., U.S. Pat. No. 9,452,285. U.S. Pat. Nos. 7,245,963, 5,273,525, 6,110,161, 6,958,060, 6,939,862, 6,697,669, 7,328,064 and US 2005/0052630

### Definitions

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the tRNA of this invention. For this purpose, one can operatively linked the nucleic acid to suitable regulatory sequences to generate an expression vector.

A vector refers to a nucleic acid molecule capable of transporting another nucleic acid to which it has been linked. The vector may or may not be capable of autonomous replication or integrate into a host DNA. Examples of the vector include a plasmid, cosmid, or viral vector. The vector includes a nucleic acid in a form suitable for expression of a nucleic acid of interest in a host cell. Preferably the vector includes one or more regulatory sequences operatively linked to the nucleic acid sequence to be expressed.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce an RNA or a polypeptide of interest. A promoter is defined as a DNA sequence that directs RNA polymerase to bind to DNA and initiate RNA synthesis. A strong promoter is one which causes RNAs to be initiated at high frequency.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for an RNA or protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development. In certain embodiments, the promoter is a PGK, CMV, RSV, HI or U6 promoter (Pol II and Pol III promoters).

A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%), or even at least 95%, 96%, 97%, 98%, or 99% sequence identity, compared to a reference sequence using one of the alignment programs described using standard parameters.

As used herein “minivector” refers to a mini-sized and circular DNA vector system, e.g., a double stranded circular DNA (e.g., a minicircle) or a closed linear DNA molecule (e.g., CEDT), lacking a bacterial origin of replication and an antibiotic selection gene, and having a size of about 100 bp up to about 5 kbp. It can be obtained, for example, by site-specific recombination of a parent plasmid to eliminate plasmid sequences outside of the recombination sites. It contains, for example, a nucleic acid molecule with merely the transgene expression cassette, including promoter and a nucleic acid sequence of interest, wherein the nucleic acid sequence may be, for example, an ACE-tRNA for e.g., suppressing PTCs, and, importantly, no bacterial-originated sequences.

The term “subject” includes human and non-human animals. The preferred subject for treatment is a human. As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). In one embodiment, the subject is a human. In another embodiment, the subject is an experimental, non-human animal or animal suitable as a disease model.

A disease or disorder associated with a PTC or nonsense mutation, PTC-associated disease, or PTC-associated disease refers to any conditions caused or characterized by one or more nonsense mutations change an amino acid codon to PTC through a single-nucleotide substitution, resulting in a defective truncated protein.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

The terms “prevent,” “preventing” and “prevention” generally refer to a decrease in the occurrence of disease or disorder in a subject. The prevention may be complete, e.g., the total absence of the disease or disorder in the subject. The prevention may also be partial, such that the occurrence of the disease or disorder in the subject is less than that which would have occurred without embodiments of the present invention. “Preventing” a disease generally refers to inhibiting the full development of a disease.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Recitation of ranges of values herein is merely intended to serve as a shorthand method of referring individually to each separate value falling within the range. Unless otherwise indicated herein, each individual value is incorporated into the specification as if it were individually recited herein.

### EXAMPLES

### Example 1 ACE-tRNA Platform

Using a novel high throughput cloning (HTC) and screening (HTS) method, a library of more than 500 suppressor tRNAs (ACE-tRNAs) was recently developed. In the library, the anticodon of human tRNA genes were engineered to recognize disease causing PTC codons (UAG, UAA and UGA). The ACE-tRNAs platform targeted all possible PTCs that are resultant of one nucleotide change from translated codons (tRNA^{Arg}_{UGA}, tRNA^{Gln}_{UAA}, tRNA^{Gln}_{UAG}, tRNA^{Trp}_{UGA}, tRNA^{Trp}_{UAG}, tRNA^{Glu}_{UAA}, tRNA^{Glu}_{UAG}, tRNA^{Cys}_{UGA}, tRNA^{Tyr}_{UAG}, tRNA^{Tyr}_{UAA}, tRNA^{Leu}_{UGA}, tRNA^{Leu}_{UAG}, tRNA^{Leu}_{UAA}, tRNA^{Lys}_{UAG}, tRNA^{Lys}_{UGA}, tRNA^{Ser}_{UGA}, tRNA^{Ser}_{UAG}, and tRNA^{Ser}_{UAA}). The ACE-tRNA^{Gly}_{UGA} (e.g., SEQ ID NO: 5) was specific for the UGA codon, which supports the premise that ACE-tRNAs have less off-target effects in vivo than molecules that generically target all three stop codons (e.g., small molecules). Furthermore, it was found that ACE-tRNA^{Gly}_{UGA} (e.g., SEQ ID NO: 5) significantly outperformed AMG G418 and gentamicin following 48 hr incubation in HEK293 cells.

Assays were carried out to ascertain whether ACE-tRNAs identified in the screen were functionalized at the expense of recognition by the aminoacyl-tRNA synthetase. Specifically, in order to be most effective, the ACE-tRNAs must suppress a PTC with the correct amino acid (cognate amino acid). It was confirmed that using high resolution mass-spectrometry, the cognate amino acid for both ACE-tRNA^{Gly}_{UGA} and -tRNA^{Trp}_{UGA} were encoded with high fidelity, resulting in seamless PTC suppression.

Assays were also carried out to determine the efficiency of ACE-tRNA dependent suppression of “real stops.” To that end, cDNA plasmids encoding ACE-tRNA^{Gln}_{UAA}, ACE-tRNA^{Glu}_{UAG}, ACE-tRNA^{Arg}_{UGA}, ACE-tRNA^{Gly}_{UGA} and ACE-tRNA^{Trp}_{UGA} (e.g., those encoded by SEQ ID NOs: 79 and 94, and those having SEQ ID NOs: 8, 5, and 1) were transfected into HEK293 cells and following 48 hrs, the cellular RNA was subjected to Ribo-Seq to determine if ribosome occupancy on the 3′ untranslated region (UTR) was higher in the presence of ACE-tRNAs compared to control (FIG. 2). FIG. 2A displays fold-change of mRNA 3′UTR ribosome occupancy of individual transcripts (dots) that have UAA (red), UAG (green) and UGA (blue). The amount of 3′UTR occupancy of ribosomes was nominal, indicating that ACE-tRNAs did not significantly suppress “real” stops. Ribo-Seq results shown in FIG. 2B allowed for visualization of average positional and magnitude of 3′UTR ribosome occupancy on all translated RNA transcripts in relationship to the stop/termination codon as a result of ACE-tRNA “real” stop suppression activity. The “sawtooth” pattern represented individual codon ribosome occupancies. The continued sawtooth pattern observed after the termination codon with ACE-tRNA^{Arg}_{UGA}, while minor, indicated in-frame readthrough. It is encouraging that the average suppression of translation termination by ACE-tRNAs is minimal following transfection of ACE-tRNAs in HEK293 cells. Off-target effects of ACE-tRNAs are being interrogated following sustained expression in transgenic 16HBE14o- cells and mice.

### Example 2 Generation of Mini DNA Vectors

Assays were carried out to examine if ACE-tRNAs can be efficiently encoded in small DNA vectors as a therapeutic deliverable.

Inventors first set out to determine how small of a MC would efficiently express ACE-tRNAs. As ACE-tRNA expression cassette was 125 bps, it was theoretically the smallest expression vector that could be generated. However, in unprecedented circumstances, the possibility that steric constraints of transcription factors and high degree of bending of DNA could inhibit ACE-tRNA expression were considered. Furthermore, generation of such small MCs less than 300 bps is hampered by the intrinsic rigidity or stiffness of the DNA double helix. Ligation-dependent circularization of linear DNA results in products predominantly containing linear concatemers, unless the reaction DNA concentrations were low (nanomolar concentrations), which impedes generation of therapeutic quantities. Maneuvers to circumvent these issues for in vitro production precludes the generation of MCs with the DNA sequence of interest and results is both labor-intensive processes and unacceptable inefficiencies. Generation of MCs in E. coli smaller than 250 bps has also been reported to be problematic due to DNA rigidity imparting low recombination efficiency. Because of technical difficulties listed above, and the fact that most expression cassettes were typically larger than 3 kb, studies using MCs smaller than a couple kilobases were few and far between, with 383 and 400 bps being the smallest published expression minivector to inventors' knowledge.

Using a recombinant mitochondrial DNA bending protein, ARS binding factor 2 protein (Abf2p), also known as Transcription Factor A, Mitochondria (TFAM, Thibault, T. et al., Nucleic Acids Res 45, e26-e26, doi:10.1093/nar/gkw1034 (2017)), inventors streamlined the production of MCs as small as 200 bps at milligram quantities in vitro (FIG. 3, method I). Here, production volumes could be easily be scaled to generate about 1.5 mgs, where production costs were significantly minimized by generation and purification of recombinant DNA ligase, DNA polymerase (PHUSION), T5 exonuclease and TFAM proteins in E. coli in inventors' laboratory. Using this method, inventors generated MCs 200-1000 bp in size (FIG. 3, Method I, bottom gel). MCs 500 bp and larger were generated in E. coli using in-house generated plasmids which utilize ϕC31 dependent recombination (FIG. 3, method II), similar to previously published methods (Kay, M. A., et al., Nat Biotechnol 28, 1287-1289, doi:10.1038/nbt.1708 (2010)).

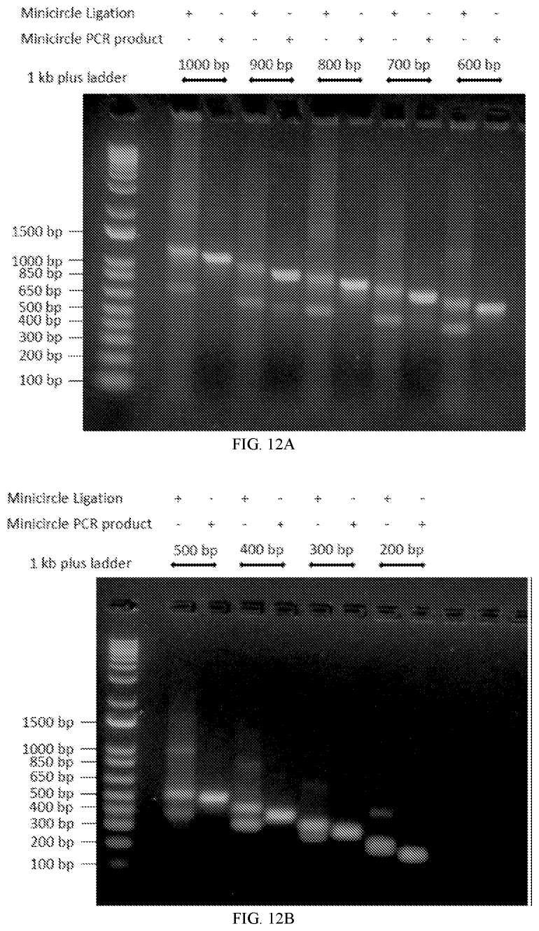

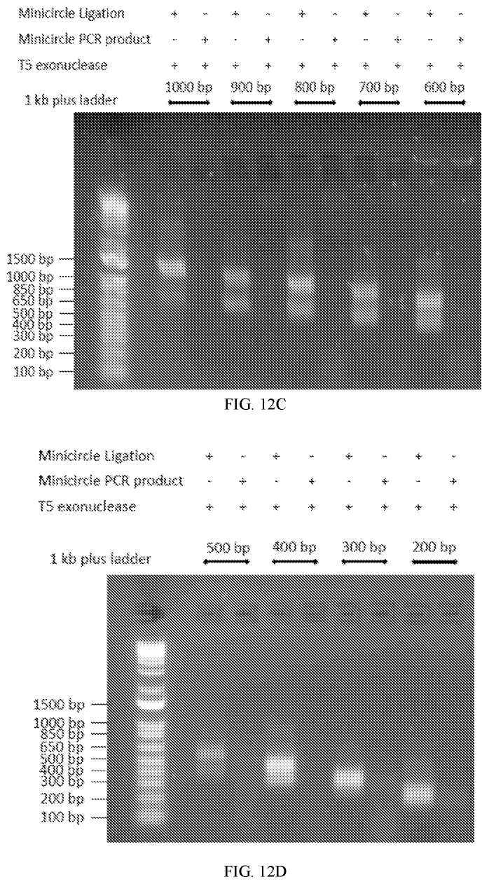

Using the methods described above, exemplary ArgTGA (e.g., encoding SEQ ID NO: 8) minicircle ligation products of different sizes were made. The sizes included about 1000 bp, 900 bp, 850 bp, 800 bp, 700 bp, 600 bp, 50 bp, 400 bp, 300 bp, and 200 bp. As shown in FIGS. 12A, 12B, 13E, and 13F, these ArgTGA minicircle ligation products and PCR products could be resolved and visualized on a 1.5% agarose gel containing ethidium bromide. The ArgTGA minicircle ligation products and corresponding PCR products were incubated with T5 exonuclease and resolved on the agarose gel containing ethidium bromide. The presence of exonuclease resistant products in the minicircle ligations indicated the production of covalently closed minicircle products (FIGS. 12C, 12D, 13E, and 13F).

MC and CEDT products can be efficiently purified away from the parent plasmid backbone by use of T5 exonuclease digestion or size exclusion chromatography (FIG. 3 Methods I, II, III & IV, bottom gels). Using these two methods inventors could be nimble with what MC ACE-tRNA sequences they want to generate, at costs reasonable for this project (˜$400 for ˜1.5 mgs of <500 bp MC and ˜$580 for 100 mg of ≥500 bp MC).

When the production of CEDTs could be achieved in several ways (Wong, S. et al. J Vis Exp, 53177-53177, doi:10.3791/53177 (2016), Schakowski, F. et al. In vivo (Athens, Greece) 21, 17-23 (2007), and Heinrich, J. et al., Journal of molecular medicine (Berlin, Germany) 80, 648-654, doi:10.1007/s00109-002-0362-2 (2002)), inventors pursued an in vitro approach using a recombinant prokaryotic telomerase, protelomerase TelN of bacteriophage N15 to reduce E. coli derived endotoxin contamination. Acting on the telomere recognition site telRL (56 bp), the protelomerase converted circular plasmid DNA into linear covalently closed dumbbell-shaped molecules in an efficient single step enzyme reaction. When two TelRL sites were inserted into an expression plasmid flanking a gene of interest, it was cleaved and joined by means of TelN enzyme (5′ end is ligated to the 3′ end), yielding linear closed end mini DNA threads encoding the gene of interest. This straightforward method was outlined in FIG. 3, Method III. Importantly, using in vitro Methods I and IV it is possible to purify large quantities of endotoxin free MC and CEDTs, respectively (Butash, K. A., et al., BioTechniques 29, 610-614, 616, 618-619, doi:10.2144/00293rr04 (2000)). Using Method III, inventors could quickly generate CEDT of varied ACE-tRNA sequences at costs reasonable for this project (˜$400 for ˜1.5 mgs of CEDT and ˜$1000 for 100 mg of CEDT).

Shown in FIGS. 13A, 13B, 13C, and 13D are the productions of four exemplary CEDTs, including a 200 bp CEDT (using a 634 bp PCR product containing two 200 bp back-to-back ArgTGA CEDT segments), a 400 bp CEDT (using a 570 bp PCR product containing a 400 bp CEDT segment), a 900 bp CEDT (using a 1065 bp PCR product encoding a 1× ArgTGA), and a 900 bp CEDT (using a 1065 bp PCR product encoding 4×ArgTGA). Each of the corresponding PCR products was purified by anion exchange chromatography (Machery Nagel kit) before being digested with TelN as described above. Once the two flanking TelRL sites were joined by TelN enzyme, the four CEDTs are 260 bp, 456 bp, 956 bp, and 956 bp, respectively. As shown in the figures, the CEDT products displayed resistance to T5 exonuclease digest indicating the production of covalently closed ends by TelN. Yet, following endonuclease cleavage by the restriction enzyme Bsu36I, each CEDT product was susceptible to degradation by T5 exonuclease.

### Example 3 Efficacy of PTC Suppression by MCs and CEDTs Encoding ACE-tRNAs

The MC and CEDT DNA vectors described in the example above were examined to validate their effectiveness at suppression of PTCs in cell culture and in vivo. Briefly, transfection of equal amounts of 200-1000 bp ACE-tRNA^{Arg} MCs (SEQ ID NO: 8, FIG. 4A, white bars) into HEK293 cells that stably expressed the PTC reporter cmv-NLuc-TGA resulted in robust PTC suppression, comparable to that of plasmid-based expression of ACE-tRNA^{Arg} (FIG. 4A, grey bar). Here, it was shown for the first time that MCs supported robust expression of ACE-tRNAs and subsequent PTC suppression. To the inventors' knowledge, these were results for the smallest functional expression MCs (<383 bps) reported.

Next, 700 and 400 bp CEDT vectors encoding ACE-tRNA^{Arg} (SEQ ID NO: 8) were transfected into 16HBE14o- cells that stably expressed the PTC reporter cmv-NLuc-UGA (FIG. 4B). Unlike MCs, while the 400 and 700 bp ACE-tRNA^{Arg} CEDTs supported strong PTC suppression (FIG. 4B, white bars), they were less efficacious than plasmid-based ACE-tRNA^{Arg}_{UGA} (FIG. 4B, grey bar) and exhibited a significant size-effect, with the 400 bp CEDT being about half as effective in PTC suppression as the 700 bp CEDT. It is unclear if this apparent effect of vector size was due to the efficiency of ACE-tRNA transcription or if this disparity was due to differences in cell-entry efficiencies with transfection-based delivery. It had been reported that DNA of different topology and size significantly influenced efficiency of delivery by different carriers.

As outlined herein, additional assays are carried out to address the influence of DNA size on cell entry by performing electroporation-based delivery of 200-1000 bp MCs and CEDTs into PTC reporter 16HBE14o- cells. With electroporation it is expected that a reduced size of DNA vector enhances cell entry, and therefore any observed decrease in suppression activity by 200-500 bp CEDTs would likely be explained by inefficient transcription imparted by DNA topology.

### Example 4 Rescue of Endogenous CFTR PTCs with ACE-tRNAs

The majority of prior studies had used suppressor tRNAs to rescue PTCs encoded from cDNAs. While informative, these studies did not present all of the hurdles encountered in vivo, such as low endogenous transcription of CFTR in lung epithelium, post-transcriptional processing/regulation of mRNA and nonsense mediated decay (NMD). In this example, 16HBE14o- cells, human bronchiole epithelial cell line, were used to determine ACE-tRNAs PTC suppression activity within the endogenous genomic landscape. Also used were mutant 16HBE14o- cells, which were CRISPR/Cas9 modified to harbor CF causing PTCs at positions p.G542X-, p.R1162X- and p.W1282X-CFTR. The cells were used to study CFTR and airway epithelia biology (Cozens, A. L. et al. American journal of respiratory cell and molecular biology 10, 38-47 (1994)).

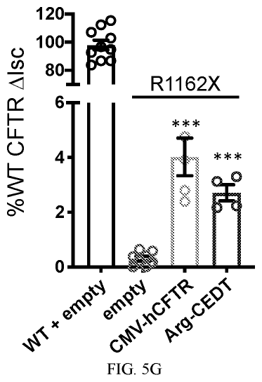

Briefly, WT 16HBE14o- cells were transfected on TRANSWELL inserts with a GFP expression plasmid to determine transfection efficiency. After 36 hrs, the cells were imaged and it was found that the transfection efficiency was lower than 10% (FIG. 5A). Despite low transfection efficiencies, Ussing chamber measurements were carried out.

First, R1162X-CFTR HBE14o- cells were transfected on Transwell inserts with a CMV-hCFTR plasmid to determine the “best-case scenario” of hCFTR functional rescue. Despite expression being driven by a strong pol II promoter, hCFTR cDNA transfection resulted in only ˜4% CFTR channel functional rescue, six days following transfection (FIG. 5G). Next the ACE-tRNA^{Arg} (SEQ ID NO: 8) 500 bp CEDT was delivered into R1162X-CFTR HBE14o- cells using the same methodology. Remarkably, ACE-tRNA^{Arg} 500 bp CEDT resulted in ˜3% rescue of CFTR function (FIG. 5G), a value similar to that of total gene replacement by a CMV-hCFTR cDNA.

As also shown in FIGS. 5B, C, and D, six days following transfection of the ACE-tRNA^{Arg}_{UGA} 500 bp CEDT into p.R1162X-CFTR 16HBE14o- cells, the inventors were surprised to measure a modest rescue of CFTR function (FIG. 5B, blue line) following addition of forskolin and IBMX (FIGS. 5B & C, ˜3% of WT) and inhibition by Inh172 (FIGS. 8B & D, 3% of WT) with no measurable CFTR function following transfection of empty vectors (FIGS. 5B, C & D, “R1162X+empty”). It should be noted that it has been estimated that as little as 10-15% rescue of CFTR function is enough to reverse CF symptoms in lung (Amaral, M. D. Pediatric Pulmonology 39, 479-491, doi:10.1002/ppul.20168 (2005)).

The inventors next looked to see if ACE-tRNAs inhibited NMD following transfection of ACE-tRNA transcripts. Interestingly, by performing transfections on plastic, it was found that the efficiencies were much higher (˜30%; FIG. 5E). qPCR was performed on mRNA isolated from p.G542X- (FIG. 5F, the second bar from the left), p.R1162X- (FIG. 5F, the fourth, fifth, and sixth bars from the left) p.R1162X- and p.W1282X-CFTR (FIG. 5F, the four bars from the right) 16HBE14o- cells two days after transfection of empty or ACE-tRNA encoding DNA vectors. It was found that CFTR mRNA expression was significantly reduced (<25% of WT). Transfection of 4×ACE-tRNA^{Gly}_{UGA} (SEQ ID NO: 5, FIG. 5F, the third bar from the left) and 4×ACE-tRNA^{Arg}_{UGA} (FIG. 5F, the fifth bar from the left with horizontal lines) plasmids resulted in a significant increase in steady state CFTR mRNA levels, about 10% and about 15% respectively. In addition, it was found that delivery of ACE-tRNA^{Arg}_{UGA} 500 bp CEDTs and 800 bp MC (FIG. 5F, the six bar and seventh bar from the left) to p.R1162X-CFTR 16HBE14o- cells resulted in a similar increase in steady-state CFTR mRNA (about 15%) as the ACE-tRNA^{Arg}_{UGA} plasmids.

Transfection of a 4×ACE-tRNA^{Trp}_{UGA} (SEQ ID NO: 1) plasmid in p.W1282X-CFTR 16HBE14o- cells did not influence CFTR mRNA steady-state expression (FIG. 5F, the third bar from the right with lines), as the ACE-tRNA^{Trp}_{UGA} was the least functioning family of the library. It had been previously shown that leucine at CFTR p.W1282 (p.W1282L) gives ˜80% WT CFTR function, as measured with Ussing chamber recordings (Xue, X. et al., Human molecular genetics 26, 3116-3129, doi:10.1093/hmg/ddx196 (2017)). Because inventors' ACE-tRNA library is a la carte in nature, they were able to pick the best 1×ACE-tRNA^{Leu}_{UGA} (SEQ ID NO: 4) plasmid and transfect them into p.W1282X-CFTR 16HBE24o- cells, which resulted in a significant increase in CFTR mRNA steady-state expression by about 17% (FIG. 5F, the second bar from the right with vertical lines).

The results presented in FIG. 5 demonstrated for the first time that ACE-tRNAs potently inhibited NMD of endogenous CFTR mRNA with CF causing PTCs, most likely through promoting pioneer round translation. Furthermore, even with rather low transfection efficiencies in Transwells, ACE-tRNAs expressed from CEDTs promoted functional rescue of endogenous CFTR in p.R1162X-CFTR 16HBE14o- cells.

### Example 5 ACE-tRNA Dependent Readthrough of PTCs In Vivo

In this example, assays were carried out to examine ACE-tRNA dependent readthrough of PTCs in vivo.

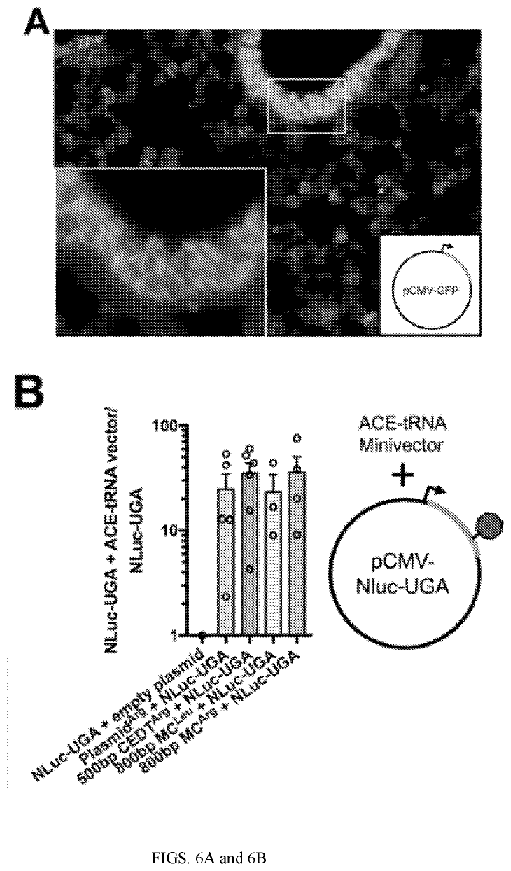

First, a cDNA plasmid encoding CMV-GFP was delivered to mouse lungs using electric fields following aspiration and assessed 3 days later by fluorescent microscopy (FIG. 6A). Quantitation of GFP expression showed that the electroporation method of gene transfer was very efficient with expression observed in 33.2%±3.5% of cells in the lung (18 to 52% in multiple sections from multiple mice) in all cell types. Importantly, GFP distribution appeared to be predominant in the airways compared to parenchyma (FIG. 6A, left inset).

Then, assays were carried out to determine if efficient delivery could be achieved with co-delivery of the PTC suppression reporter plasmid pNanoLuc-UGA and vectors encoding ACE-tRNAs. Control mice received the PTC reporter pNLuc-UGA plasmid to determine spurious PTC readthrough. Two days following the electroporation, the lungs were dissected, perfused with saline, and homogenized with lysis buffer (PROMEGA) in a bead-beater apparatus. Lysates were spun at high speed and the supernatant was analyzed for NLuc activity using a plate-reader. In the absence of ACE-tRNA vectors, no significant luminescence was measured (FIG. 6B, the first bar from the left). It was found that ACE-tRNAArg (SEQ ID NO: 8) plasmid (FIG. 6B, the second bar from the left), 500 bp ACE-tRNAArg CEDT (FIG. 6B, the middle bar), 800 bp ACE-tRNALeu (SEQ ID NO: 4) MC (FIG. 6B, the second bar from the right) and 800 bp ACE-tRNAArg MC (FIG. 6B, the first bar from the right) supported NLuc-PTC rescue in mouse lung in vivo. These results provide the proof-of-principle for (i) the effectiveness and persistence of nonsense suppression by ACE-tRNA encoding plasmids, MCs and CEDTS in airway epithelia of p.G542X-CFTR and p.W1282X-CFTR mice and (ii) the efficiency and persistence of ACE-tRNAs encoded in MCs and CEDTs following delivery into lungs.

### Example 6 Identifying TELS

This example describes assays for identifying TELS to enhance ACE-tRNA expression from tRNA 5′ flanking sequences. The tRNA 5′ flanking sequences (about 1 kb) from tRNA genes, such as one or more of the 416 tRNA genes descried in Lowe et al., Nucleic Acids Res 25, 955-964, doi:10.1093/nar/25.5.955 (1997), can be used.

Each sequence is cloned into an all-in-one cDNA plasmid that supports both high-throughput cloning (HTC) and quantitative high-throughput screening (HTS) of PTC suppression using luminescence following delivery to mammalian cells (FIG. 7B). The 1 kb 5′ flanking sequences are cloned as gBlocks (Integrated DNA Technologies) in 96 well format into the HTC site using GOLDEN GATE cloning, paired with ccdB negative selection to give about 100% cloning efficiency. All clones are confirmed by Sanger sequencing. The TEL sequences are cloned immediately 5′ of ACE-tRNA^{Arg}_{UGA} (SEQ ID NO: 8, FIG. 7B), and NLuc-UGA suppression efficiency is read out using a plate-reader in 96-well fashion, where increased PTC suppression indicates increased ACE-tRNA^{Arg}_{UGA} expression. After the first screen is completed, the top ten 1 kb sequences are split into 250 bp sequences to identify the origin of transcription enhancement, again by cloning gBlock sequences. 1 kb leader sequences that enhance or inhibit ACE-tRNA transcription are analyzed for sequence motifs using numerous developed software tools (Sharov, A. A. & Ko, M. S. H. DNA Research 16, 261-273, doi:10.1093/dnares/dsp014 (2009))¹⁰⁶. It is expected that one or more TELS can enhance the expression of ACE-tRNAs by several fold, to allow less efficient delivery and maintain potent PTC suppression. Results from this study also provide insight into regulation of tRNA transcription.

### Example 7 Identifying DTSs

This example describes assays for identifying DTSs, which drive minivector nuclear localization.

Several DNA sequences that target plasmids into the nuclei of non-dividing cells have been identified. See, e.g., Dean, D. A. Exp. Cell Res. 230, 293-302 (1997), Dean, D. A., et al., Exp. Cell Res. 253, 713-722 (1999), Vacik, J., et al., Gene Therapy 6, 1006-1014 (1999), Young, J. L., et al., Mol. Biol. Cell 10S, 443a (1999), Langle-Rouault, F., J Virol 72, 6181-6185 (1998), Mesika, A., et al., Mol Ther 3, 653-657. (2001), Degiulio, J. V., et al., Gene Ther, doi:gt2009166 [pii] 10.1038/gt.2009.166 (2010), Sacramento, C. B., et al., Brazilian journal of medical and biological research 43, 722-727 (2010), and Cramer, F. et al., Cancer Gene Ther 19, 675-683, doi:10.1038/cgt.2012.54 (2012). The common feature to these sequences is that they contain binding sites for transcription factors. Interestingly, the SV40 enhancer acts as a DTS in all cell types due to its binding of >10 ubiquitously expressed transcription factors that contain nuclear localization sequences (NLSs). A typical transcription factor would be transported into the nucleus, bind to its regulatory DNA target sequence and activate or repress transcription. However, if DNA containing the transcription factor binding site is present in the cytoplasm, the cytoplasmic transcription factor may bind to this site before nuclear import (FIG. 8) and translocate the DNA-protein complex into the nucleus.

Inventors have screened over 60 strong general and cell-specific promoters and found seven DTSs. Of these, two perform ubiquitously, while five bind specific transcription factors expressed in a subset of cells. It was found that incorporation of DTSs into expression plasmids increases gene expression in microinjected and transfected non-dividing cells, and important for this invention act to increase nuclear targeting of plasmids and subsequent gene expression in vivo. Here the inventors have shown that the minivector parent pUC57 plasmid encoding ACE-tRNAs is not transported into the nuclei of nondividing cells following cytoplasmic microinjection. To improve plasmid nuclear localization and subsequent expression of the ACE-tRNAs, one can include the SV40 DTS into the plasmid.

One can also carry out a screen to identify new DTSs that are more effective than the SV40 DTS at nuclear targeting to improve ACE-tRNA minivector delivery, expression, and ultimately, nonsense suppression. Since it is preferred to identify DTSs that act in all cell types, one can screen promoters that are ubiquitously expressed. A number of databases of housekeeping genes identified by microarray, RNAseq, and single cell sequencing have been published in e.g., Curina, A. et al., Genes Dev 31, 399-412, doi:10.1101/gad.293134.116 (2017), Eisenberg, E. & Levanon, E. Y. Trends Genet 19, 362-365, doi:10.1016/50168-9525(03)00140-9 (2003), and Eisenberg, E. & Levanon, E. Y. Trends Genet 29, 569-574, doi:10.1016/j.tig.2013.05.010 (2013).

Promoter sequences for the top 20 house-keeping genes can be identified based on published sequence or bioinformatic analysis and identification of the transcriptional start sites for each promoter from the DBTSS data set (dbtss.hgc.jp). These promoters typically range in size from 500-2000 bp and can be cloned by PCR from human genomic DNA into a reporter plasmid that expresses GFP from the CMV promoter (which does not function as a DTS). This plasmid also can carry a binding site for a triplex-forming peptide nucleic acid (PNA) to enable fluorescent labeling of the DNA. By hybridizing Cy3-labeled PNAs to this site on the plasmid, one can generate high quantum yield, fluorescently labeled plasmids to follow nuclear import in real time (Gasiorowski, J. Z. & Dean, D. Mol Ther 12, 460-467 (2005)).

As an alternative approach to directly labeling the plasmid with Cy3-PNAs, one can also carry out fluorescent in situ hybridization (FISH) to visualize injected DNA. DTS candidate plasmids can be tested for nuclear import activity over a 30 min to 8 hr period in microinjected A549 lung epithelial cells (Dean, D. A. Exp. Cell Res. 230, 293-302 (1997), Dean, D. A., et al., Exp. Cell Res. 253, 713-722 (1999), and Vacik, J., et al., Gene Therapy 6, 1006-1014 (1999)). While the inventors have found that the SV40 sequence mediates plasmid nuclear import within 30 min, the SMGA promoter needs 4 hrs for plasmids to localize to the nucleus in smooth muscle cells. This approach can allow one to rank sequences relative to the SV40 DTS based on their speed of nuclear import. Negative controls of the pUC57 plasmid with no DTS and a positive control of the pUC57-SV40 DTS can also be injected to ensure that the cells remain viable after injection and have the ability to transport DNA into the nucleus.

To test cell-specificity, one can microinject candidate DTS containing plasmids into multiple cell types, including HEK293, primary smooth muscle and endothelial cells, and human fibroblasts. Based on inventors' experience, it can be predicted that one can identify two to four general DTS from this screen. Once having identified promoter sequences that show import activity, one can create a limited number of truncated promoters (e.g., three to four) to identify the minimal sequence length that supports nuclear targeting in the manner described in Miller, A. M. & Dean, D. A. Gene Ther 15, 1107-1115 (2008), Degiulio, J. V., et al., Gene Ther 17, 541-549, doi:10.1038/gt.2009.166 (2010), and Gottfried, L., et al., Gene Ther 23, 734-742, doi:10.1038/gt.2016.52 (2016). One can next determine whether these minimal DTSs also support plasmid nuclear targeting in vivo following delivery to the lungs of mice by electroporation and quantifying nuclear delivery of the plasmids by IF for eGFP expression in thin sections of the lungs in the manner described in Dean, D. A., et al., Gene Ther 10, 1608-1615 (2003), Machado-Aranda, D. et al. Am J Respir Crit Care Med 171, 204-211 (2005), Mutlu, G. M. et al., Am J Respir Crit Care Med 176, 582-590 (2007), Zhou, R., et al., Gene therapy 14, 775-780, doi:10.1038/sj.gt.3302936 (2007), Young, J. L., et al., Methods Mol Biol 1121, 189-204, doi:10.1007/978-1-4614-9632-8_17 (2014), and Young, J. L. et al., Adv Genet 89, 49-88, doi:10.1016/bs.adgen.2014.10.003 (2015). As controls, reporter plasmids carrying no DTS or the SV40 DTS are also transferred to mouse lungs.

It is anticipated that two to four promoters show general DTS activity in cultured cells. Thus, one can test four potential DTSs and two controls in mice (n=6). One can also test this separately in male and female C57B6 mice and repeat the experiment once. Two days after gene transfer, animals are euthanized, and lungs are perfused, fixed in 4% paraformaldehyde, run through sucrose solutions, and embedded in OCT for frozen thin sectioning. GFP positive cells are counted along with each cell-specific marker to determine the number of GFP positive cells for each cell type. Antibodies for Acetylated-tubulin (ciliated airway epithelial cells), CCSP (Club cells), Muc5AC (goblet cells), CD31 (endothelial cells), and SMAA (smooth muscle) can be used to identify lung cell types. The inventors have used this approach in the past to determine cell-specificity of nuclear import. The best sequences can then be incorporated into our ACE-tRNA minivectors to maximize delivery and expression. Importantly, DTSs identified can be applied directly to all DNA therapeutic approaches to enhance nuclear targeting and therapeutic transgene expression.

### Example 8 MCs and CEDTs with of DTS and TELS

In this example, assays are carried out to examine if the incorporation of DTS and TELS to 500 bp ACE-tRNA^{Arg}_{UGA} (SEQ ID NO: 8) MCs and CEDTs improves nuclear targeting, transcription and PTC suppression.

Importantly, existing MC and CEDT technologies are efficient at PTC suppression in both cell culture and in vivo (FIGS. 4-7 and 10). That being said, tRNA transcription factors (Pol III transcription elements) did not escort the ACE-tRNA^{Arg} MCs into the nucleus (FIG. 8B) following cytoplasmic injection. Therefore, to improve nuclear targeting, transcription and PTC suppression, ACE-tRNA^{Arg}_{UGA} MCs and CEDTs with the 72 nucleotide SV40 DTS and generally active DTS are generated. Cellular cytoplasmic and nuclear injections of these vectors into 16HBE14o- cells are carried out in the same manner described herein.

DNA localization is determined using FISH 4 hrs later. Also performed is a limited mouse lung electroporation study with CEDTs and MCs with and without DTSs co-electroporated with the pNLuc-UGA PTC reporter plasmid (CEDT, n=8; CEDT-DTS, n=8; MC, n=8; MC-DTS, n=8; 4 male and 4 female) as outlined below. The SV40 DTS and the 4 DTSs are tested for a total of 5 DTS per MC or CEDT (use 96 mice). It is hypothesized that the addition of the various DTS greatly enhances ACE-tRNA transcription by increasing its nuclear bioavailability which subsequently enhances PTC suppression. It is predicted that inclusion of the DTS modification can result in more than 2-fold PTC suppression in in vivo electroporation studies.

### Example 9 Ability of Minivectors to Suppress PTCs in Cells

This example describes studies to determine the ability of minivectors to suppress PTCs in 16HBE14o- cells (p.G542X, p.R1162X and p.W1282X). The delivery method used here is the LONZA 4D-NUCLEOFECTOR™ X System where solution “SG” and program CM-137 have already been optimized for 16HBE14o- cells.

The influence of vector size on ACE-tRNA CEDTs and MC PTC suppression in p.R1162X-CFTR 16HBE14o- cells are interrogated by performing a screen of 125, 200, 300, 400, 500, 600, 700, 800, 900 and 1000 bp DNA minivectors. Rescue of p.R1162X-CFTR function is determined using Ussing Chamber recordings (n=4-6) and subsequent qPCR analysis to determine steady-state CFTR mRNA expression (n=4-6). For Ussing chamber recordings, inventors employ a Cl−gradient (140 mM Cl−Basolateral/4.8 mM Cl−) supplemented with 5 mM dextrose and subsequent addition of amiloride (100 μM) to block ENaC and DIDs (100 μM) to block CaCC activity. CFTR are subsequently activated by forskolin (10 μM) and IBMX (3-Isobutyl-1-methylxanthine, 100 μM) and inhibited by 10 μM Inh172. Rescue of CFTR activity is analyzed as ΔI_{Cl}- of pre- and post-F&I and pre- and post-Inh172 block as described in FIG. 5B.

The optimal minivector conditions are used to test the function MCs and CEDTs encoding ACE-tRNA^{Leu}_{UGA} (SEQ ID NO: 4) delivered to p.W1282X 16HBE14o- cells. The top DTS and TEL sequences identified in examples above are included in the optimally sized minivectors. It is expected to increase the PTC suppression ability of the minivectors several fold.

### Example 10 ACE-tRNA Expressing Vectors Suppress CFTR p.G542X and p.W1282X in Mouse

This example describes studies to examine the ability of ACE-tRNA encoding cDNA plasmids, CEDTs and MCs to suppress CFTR p.G542X and p.W1282X in mouse lungs. Mouse models with p.G542X- and p.W1282X-CFTR PTC mutations have been generated. For example, the p.G542X mouse was published in McHugh, D. R., et al., PloS one 13, e0199573, doi:10.1371/journal.pone.0199573 (2018). These p.G542X- and p.W1282X-CFTR mice are used for studying PTC therapeutics as the changes in CFTR transcripts closely recapitulate human.

The electric field (electroporation) method is utilized for delivering the DNA vectors to lung using method know in the art. See, e.g., Dean, D. A., et al., Gene Ther 10, 1608-1615 (2003), Zhou, R. & Dean, D. A. Experimental biology and medicine (Maywood, N.J.) 232, 362-369 (2007), O'Reilly, M. A. et al. The American journal of pathology 181, 441-451, doi:10.1016/j.ajpath.2012.05.005 (2012), Mutlu, G. M. et al., American journal of respiratory and critical care medicine 176, 582-590, doi:10.1164/rccm.200608-12460C (2007), Blair-Parks, K., et al., The journal of gene medicine 4, 92-100 (2002), and Barnett, R. C. et al., Experimental biology and medicine (Maywood, N.J.) 242, 1345-1354, doi:10.1177/1535370217713000 (2017). Here cDNA plasmid and 500 bp CEDTs and MCs are delivered to lungs of 3-week-old (weaned) homozygous CFTR p.G542X (ACE-tRNA^{Gly}_{UGA}) and p.W1282X (ACE-tRNA^{Leu}_{UGA}) mice by electroporation. A cDNA Plasmid (2.7 kb puc57 parent plasmid for both MC and CEDT) encoding a scrambled tRNA sequence is used as control for effects of electroporation and possible effects of expressed ACE-tRNAs on lung cell viability. Briefly, 50 ul of DNA (2 mg/ml) in salt-balanced solution is aspirated into the mouse lungs. Immediately following delivery, a series of 8×10 msec square wave electric pulses (200 V/cm) is administered to the animals using pediatric cutaneous pacemaker electrodes (MEDTRONICS) while isoflurane is administered. The electrodes are placed on either side of the chest using a small amount of surgical lubricant to aid conductance.

Mice are analyzed 7, 14, and 21 days (d) following delivery. Because only 40% of homozygous p.G542X- and p.W1282X-CFTR mice survive to 40 d of age due to intestinal obstruction, the study starts with large cohorts of mice (n=12) to achieve n=6 for each group. In total, a minimum of 384 mice are used to complete this study. Both sexes are used to achieve adequate experimental animals. At each time point mice are euthanized, and the lungs removed and separated into right and left lobes. The right lobe is perfused and inflation-fixed with 4% paraformaldehyde/30% sucrose prior to embedding in OCT for frozen sections. The left lobe is flash frozen for protein (Western Blot, WB) and RNA isolation. The left lung lobe is crushed under liquid nitrogen and ⅛^{th} of the lung powder is analyzed for CFTR mRNA. The remaining lung is dounce homogenized in buffer containing 150 mM NaCl, 50 mM Tris pH 8.0, and supplemented with protease inhibitors and 2% CHAPS (w/v). Glycosylated CFTR protein is affinity purified using wheat germ agglutin (WGA) bound agarose beads, washed and eluted with 200 mM N-acetylglucosamine (NAG). Samples are immunoblotted using anti-CFTR antibody (1:1000; M3A7, MILLIPORE, USA). Intestine from mice serves as untreated tissue controls for WB and qPCR analysis. Sections of the right lobe are cut from three levels in the lung, representing the top, middle, and bottom, and stained by FISH (Cy5 or Cy3) towards the minivector and DAPI (FIG. 8). Airway cells are identified by co-staining with specific antibodies in serial sections to identify which cells are transfected. Quantitation is performed by counting the numbers of FISH+ cells/total cells in 10 sections from each of the levels of the lung.

Antibodies used for co-staining include keratin 5 (basal cells in submucosal glands), acetylated-tubulin and FoxJ1 (ciliated or non-ciliated airway epithelial cells depending on staining), nerve growth factor receptor, keratin 14, p63 (basal cells in airways), and Muc5AC (goblet cells). CFTR is detected by immunohistochemistry and immunofluorescence using mouse monoclonal CFTR-769. Because electroporation is extremely efficient at delivering DNA to airway epithelial cells (FIG. 6A), inventors predict significant rescue (>10%) of CFTR protein in airway epithelial cells, levels high enough to detect by IF.

Inflammatory cytokine levels are measured in a subset of mice (n=6) before and immediately after electroporation, and then again at the time of lung harvest (7d timepoint) to determine if treatment caused an inflammatory response. Importantly, it was previously shown no inflammatory response in pigs or mice following electroporation. Hematoxylin and eosin (H&E) histological analysis is performed to detect changes in lung morphology.

### Example 11 Persistence of ACE-tRNAArgUGA Expressing Vectors in WT Mouse Lung

This example describes studies to determine the persistence of ACE-tRNA^{Arg}_{UGA} (SEQ ID NO: 8) expressing plasmid, CEDTs and MCs in WT mouse lung.

Because very few p.G542X- and p.W1282X-CFTR mice survive past 40d of life, likely it may not be possible to determine the full persistence of ACE-tRNA expression from CEDTs and MCs in Example 10. The inventors and others have demonstrated transgene expression in mouse lung >6 months. The inventors therefore deliver ACE-tRNA^{Arg}_{UGA} encoding cDNA plasmids (2.7 kb puc57 parent plasmid for both MC and CEDT), 500 bp CEDTs and MCs to the lungs of 3-week-old C57Bl/6 male WT mice as previously described in Example 10) and determine their delivery efficiency and persistence and PTC suppression efficiency. The parent plasmid encoding a scrambled tRNA sequence are used as control for effects of electroporation and possible effects of expressed ACE-tRNAs on lung cell viability at 7 days, 14 days, and 1, 2, 6, and 12 months.

The study starts with 10 mice per treatment (5 male/5 female) and endpoint is used for this portion of the study, for a total of 240 mice. Three days before each endpoint, a subsequent delivery of a small (3 kb) PTC reporter cDNA plasmid with a SV40-DTS for nuclear targeting that expresses an NLuc-UGA protein with an n-terminal hemagglutinin (HA) epitope tag and c-terminal FLAG epitope tag under control of a short Ubiquitin C promoter (shUbC) (FIG. 9) is made.

At defined endpoints the lungs are removed and treated as described above for IF and FISH analysis (right lobe) and protein biochemistry (left lobe). The PTC reporter plasmid is multifunctional, with a sequence that is completely different from minivectors and can therefore be identified by co-FISH to determine co-delivery efficiency of plasmids. When the minivectors and PTC reporter plasmid co-localize, readthrough efficiency can be determined by NLuc luminescence using in vivo imaging, plate reader measurements of explanted lung tissue (FIG. 6B) or biochemically using the c-terminal FLAG tag (left lobe). Expression of the NLuc protein can be followed by IF or WB using the n-terminal HA epitope tag, and only when the PTC is suppressed will there be appreciable FLAG signal. Importantly, this DNA construct is similar to that used in our high-throughput screen to identify the best ACE-tRNA sequences for PTC suppression. It has been modified to give high signal to noise by reducing background readthrough to faithfully report bone fide PTC suppression.

The study has 5.5 g endotoxin free PTC reporter cDNA plasmid generated (ALDEVRON) in one batch to ensure reproducibility for experiments outlined here in mice. It is predicted that delivery of 500 bp minivectors is more efficient than the 3 kb PTC reporter plasmid (as determined by co-FISH labeling of cells) and therefore give an underestimate of minivector PTC suppression activity. The inventors use FISH towards the minivectors to determine persistence of minivector presence in lungs at each endpoint, and luminescence and IF towards the PTC reporter c-terminal FLAG epitope are used quantify persistence of suppression action. IF antibodies and techniques detailed in Example 10 paired with FISH are used to identify what cell types are transduced.

To make CFTR functional measurement, tracheas are removed and split in half longitudinally as described in Grubb, B. R., et al., Am J Physiol 267, C293-300, doi:10.1152/ajpcell.1994.267.1.C293 (1994), 160 Grubb, B. R. et al. Nature 371, 802-806, doi:10.1038/371802a0 (1994), and Grubb, B. R., et al., Am J Physiol 266, C1478-1483, doi:10.1152/ajpcell.1994.266.5.C1478 (1994). Half is mounted for analysis in Ussing chambers (PHYSIOLOGIC INSTRUMENTS) with a 2 mm diameter aperture chamber and short circuit currents are measured in the manner described in Cooney, A. L. et al., Nucleic Acids Res 46, 9591-9600, doi:10.1093/nar/gky773 (2018), Cooney, A. L. et al., JCI Insight 1, doi:10.1172/jci.insight.88730 (2016), Mall, M., et al., Nat Med 10, 487-493 (2004), and Zhou, Z. et al. J Cyst Fibros 10 Suppl 2, S172-182, doi:10.1016/51569-1993(11)60021-0 (2011). The difference in short-circuit current is calculated after the addition of 10 μM forskolin and 100 μM IBMX to the basolateral side of the tracheal sections followed by 10 μM Inh172, similar to experiments performed in FIG. 7B-D. Using the remaining tracheal section, one can determine CFTR expression in the airways by immunofluorescence using the CFTR769 antibody against the NBD2 domain. Importantly, excised portions of the lungs are banked for future RNAseq and Riboseq studies.

For some experiments described herein, the inventors rely on PTC suppression plate-reader luminescence assay to extrapolate persistent expression of ACE-tRNAs. The goal is to create a technology that allows direct measurement of ACE-tRNA transcription activity. To that end, the inventors designed vectors to generate ACE-tRNA:barcode fusion transcripts, where the barcode sequence (ABS) is cleaved by the endogenous tRNase Z, allowing for qPCR quantification to determine relative ACE-tRNA expression, while giving a fully functional ACE-tRNA (FIG. 10A).

The inventors designed a random 200 bp barcode sequence with no homology to mouse genomic sequence. When transfected into 16HBE14o- cells that stably express NLuc-UGA, ACE-tRNA^{Arg}_{UGA}- and ACE-tRNA^{Trp}_{UGA}-Barcode exhibit robust expression as monitored by qPCR, while the non-barcoded ACE-tRNA gave no significant signal (FIG. 10B). However, in one case, the suppression activity of the ACE-tRNA^{Arg}_{UGA} was impeded by the presence of the 3′ barcode sequence (FIG. 10C). The deficit in ACE-tRNA activity most likely due to poor 3′ processing, which can be determined by northern blot probed towards the barcode sequence. Work can be done to improve the ABS technology by encoding a 3′ HDV self-cleaving ribozymes whose sequence will also act as a barcode or modified barcode linker sequence to improve 3′ processing. Incorporation of 3′ ribozymes can result in improved ACE-tRNA 3′ processing and increase their PTC suppression activity. Furthermore, the ABS technology allows measurements of ACE-tRNA expression to compliment the NLuc PTC reporter (FIG. 9).

In another embodiment, the inventors created a novel ACE-tRNA barcode system technology that allows high resolution straightforward measurements of steady state ACE-tRNA transcription activity (FIG. 11A). Standard RNA-seq methods cannot be implemented with tRNAs because of extensive post transcriptional modifications and secondary structure. Zheng, G. et al., Nature Methods 12, 835 and Pang et al., Wiley Interdiscip Rev RNA 5, 461-480, doi:10.1002/wrna.1224 (2014). Furthermore, the ACE-tRNA sequences differ by only one nucleotide from one or more endogenous tRNAs, therefore northern blot, microarray and fragmented RNA-seq cannot discern expression level of exogenous therapeutic ACE-tRNAs and endogenous tRNAs. Inventors therefore generated the barcode technology to sidestep these issues and provide reliable straightforward and sensitive qRT-PCR measurement of ACE-tRNA transcription. Here, the unique 3′ barcode sequence encodes a HDV ribozyme (drz-Bflo-2, 60 bp). (Webb et al., Science 326, 953 (2009) and Webb et al., RNA Biol 8, 719-727 (2011)) that efficiently cleaves itself (>90%) from the ACE-tRNA body after transcription (FIG. 11B). The HDV expression that is driven by the ACE-tRNA transcription is quantified using probe based qRT-PCR. Importantly, the ACE-tRNA expressed in barcoded vectors are unable to participate in translation due to 3′ terminal 2′,3′ cyclic phosphate group modification (Schürer et al., Nucleic Acids Res 30, e56-e56, 2002), and therefore do not confound PTC suppression results.

### Example 12 Rescue of Endogenous CFTR PTCs with G418 and ACE-tRNAs

In this example, studies were carried out to examine and compare rescues of endogenous CFTR PTCs with a conventional PTC readthrough agent (i.e., G418) and ACE-tRNAs delivered in plasmids. To that end, an engineered human bronchiole epithelial cell line, 16HBE14ge-, was used (Valley et al., Journal of Cystic Fibrosis, Volume 18, Issue 4, July 2019, Pages 476-483). G418 (Geneticin) is a standard PTC readthrough agent that works by interacting with the decoding center of the ribosome and promoting the suppression of PTCs with near-cognate tRNAs, thus incorporating wrong amino acids in many instances. Due to its non-specific nature, rescue by G418 results in generation of a missense mutation from a nonsense mutation. In contrast, ACE-tRNAs as disclosed herein put in the desired amino acid.

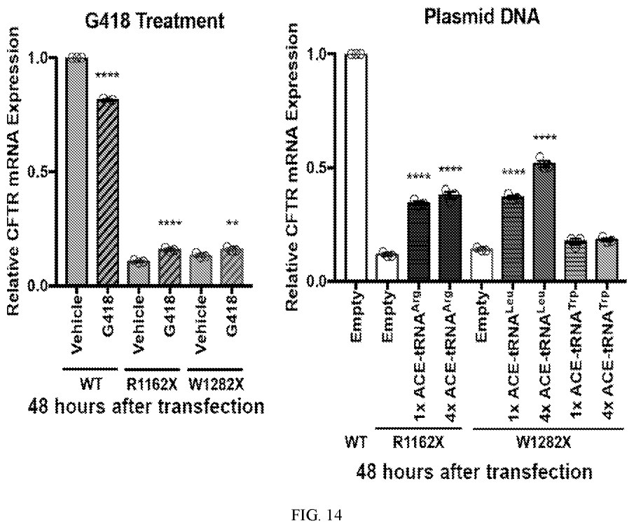

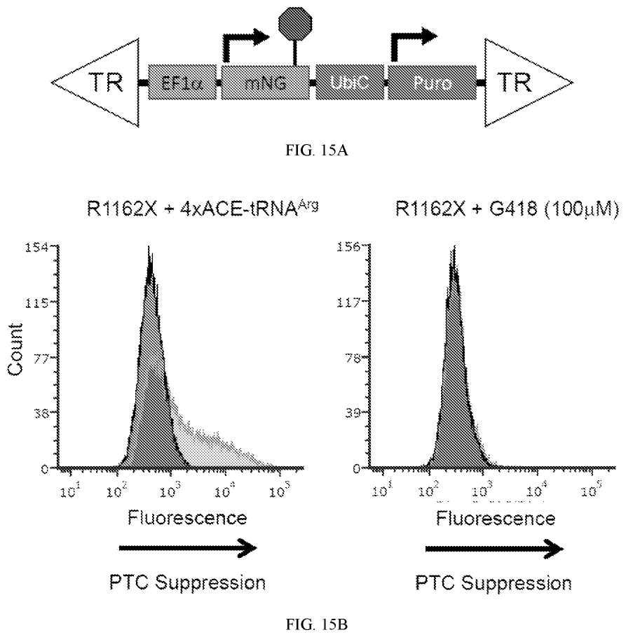

To carry out the assays, wildtype 16HBE14ge- cells or 16HBE14ge- cells having the W1282X-CFTR or R1162X-CFTR mutation were (i) treated with 100 uM G418 or vehicle, or (ii) transfected with empty plasmids or various plasmids encoding one or four copies of three ACE-tRNAs (SEQ ID NOs: 8, 4, and 1): 1×ACE-tRNA^{Arg} 4×ACE-tRNA^{Arg}, 1×ACE-tRNA^{Leu} 4×ACE-tRNA^{Leu}, 1×ACE-tRNA^{Trp}, and 4×ACE-tRNA^{Trp} in the manner described in Example 4. After 48 hours, the corresponding CFTR mRNA expression levels were then determined. The results are shown in FIG. 14.

As shown in the figure, the ACE-tRNAs encoded from plasmids and transfected into 16HBE14ge- cells were significantly more effective in inhibiting nonsense mediated decay processes than G418 through promoting the pioneer round of translation with the use of Arginine and Leucine ACE-tRNAs. An important aspect of this data also is that the platform disclosed herein supports a la carte PTC suppression. Unexpectedly, it was found that Leucine ACE-tRNAs suppressed W1282X more effectively than Tryptophan ACE-tRNA. Since Leucine at position W1282X supports WT levels of CFTR function (Xu et al., Hum Mol Genet. 2017 Aug. 15; 26(16):3116-3129), the ACE-tRNA^{Leu} described herein can be used to rescue or suppress the mutation pf W1282X-CFTR.

As PTCs result in both (a) truncated protein with complete loss of or altered function, and (b) degradation of the mRNA transcript by NMD pathways, additional assays were designed and carried out to examine effects ACE-tRNAs on both fronts. More specifically, 16HBE14ge- cells were generated to express a PTC readthrough reporter piggyBac transgene as shown in FIG. 15A. As the transgene encoded a mNeonGreen (mNG) protein having a PTC, suppression of the PTC by ACE-tRNAs can be quantitated by FACs sorting based on fluorescence from expressed mNG protein. To that end, an increase in fluorescence represents PTC suppression at the level of translation.

These cells were treated with 100 uM G418 or transfected with plasmids encoding 4×ACE-tRNA^{Arg} in the manner described above. The cells were then examined for fluorescence from expressed mNG protein. The results are shown in FIG. 15B. As shown in the figure, the Arginine ACE-tRNA supported robust PTC suppression and generation of full length mNeonGreen protein. In contrast, G418 did not support robust PTC suppression. The grey profile was for non-treated PB-mNeonGreen-R1162X-16HBE14ge- cells.

### Example 13 Rescue of Endogenous CFTR PTCs with ACE-tRNAs Delivered in Different Formats

In this example, assays were carried out to examine and compare rescues of endogenous CFTR PTCs with ACE-tRNAs delivered in different formats e.g., plasmids, CEDTs, and MCs. The above described 16HBE14ge- cells, which expressed the PTC readthrough reporter piggyBac transgene (FIG. 15A) was used. More specifically, the cells were transfected with plasmids, CEDTs, or MCs encoding ACE-tRNAs. The cells were then sorted by FACS and mRNA was isolated from the non-green cells and green cells. Targeted RT-qPCR was used to quantify CFTR mRNA expression.

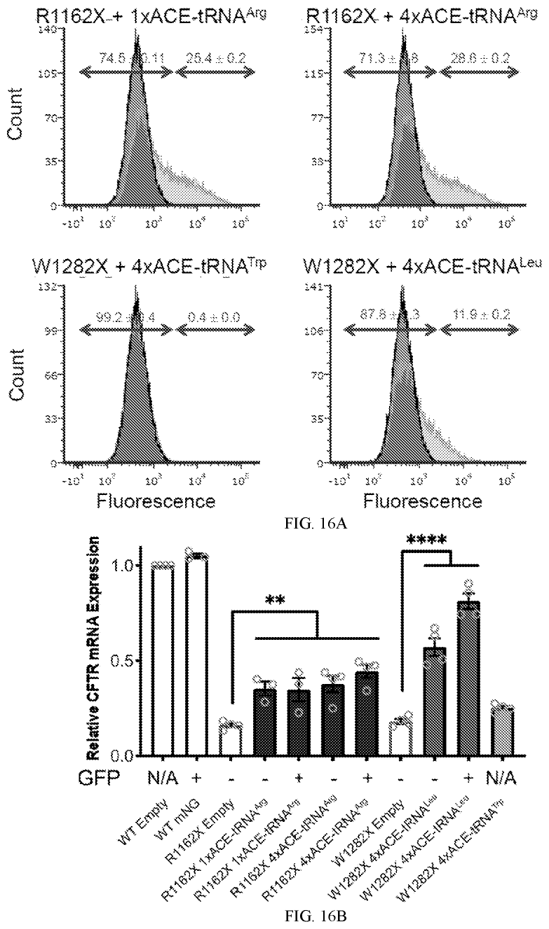

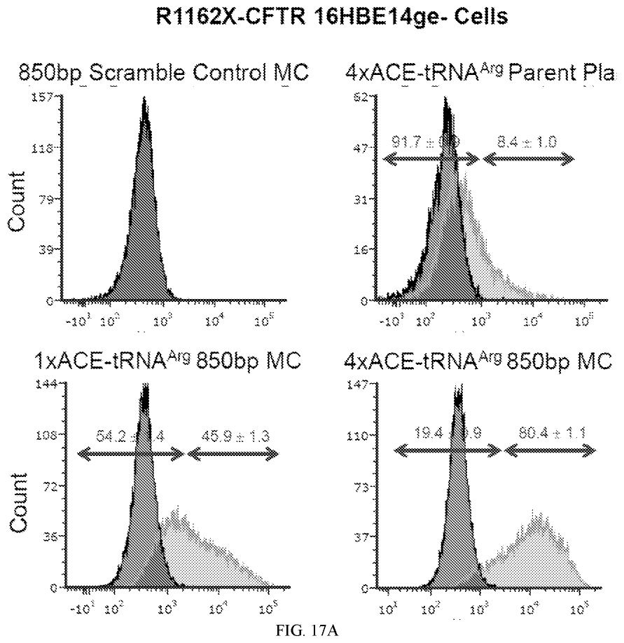

In some essays, the cells were transfected with plasmids encoding ACE-tRNA^{Arg} and ACE-tRNA^{Leu} (SEQ ID NOs: 8 and 4). The results were shown in FIGS. 16A and B. As shown in FIG. 16B, both ACE-tRNA^{Arg} and ACE-tRNA^{Leu} delivered via plasmids significantly rescued CFTR mRNA from R1162X and W1282X 16HBE14ge- cells, respectively. Such CFTR mRNA rescues were observed in both green cells (GFP+) and non-green cells (GFP−). The reason cells that were not green still had rescue of CFTR mRNA expression was because the ACE-tRNA was effectively rescuing mRNA expression through pioneer round translation but not significant levels of translation. These results suggested that NMD inhibition was relatively easier to achieve than robust translation rescue.

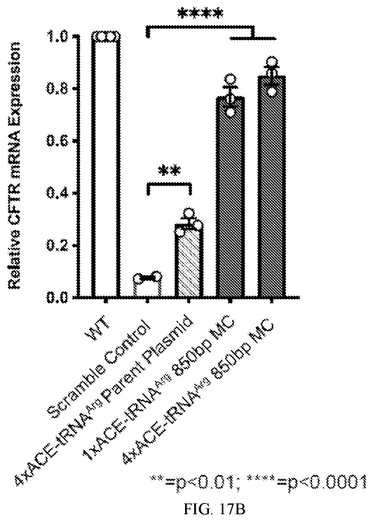

In other assays, 16HBEge- cells having R1162X-CFTR and the PTC readthrough reporter piggyBac transgene were transfected with minicircles encoding the ACE-tRNAs by electroporation. These cells were NOT sorted by FACS for mRNA expression analysis, rather the entire population was analyzed. As shown in FIG. 17A, the ACE-tRNA encoding minicircles supported robust mNeonGreen expression in the majority of cells. These results indicated that ACE-tRNA expressing minicircles supported PTC suppression to a level that could be quantitated at the protein level. This rescue was significantly better than the parent plasmid (˜7.0 kb in size) and a scramble control. Also, MCs of 850 bp encoding one copy of ACE-tRNA (“1×ACE-tRNA^{Arg} 850 bp MC”) and MCs of 850 bp encoding four copies of ACE-tRNAs (“4×ACE-tRNA^{Arg} 850 bp MC”) led to similar levels of CFTR mRNA expression (FIG. 17B). Yet, the latter resulted in much more mNeonGreen-expressing cells (FIG. 17A, bottom two panels). These results suggested that increasing the amount of ACE-tRNAs did not seem to increase NMD inhibition, but increased protein generation. This level of mRNA expression rescue was unprecedented and novel.

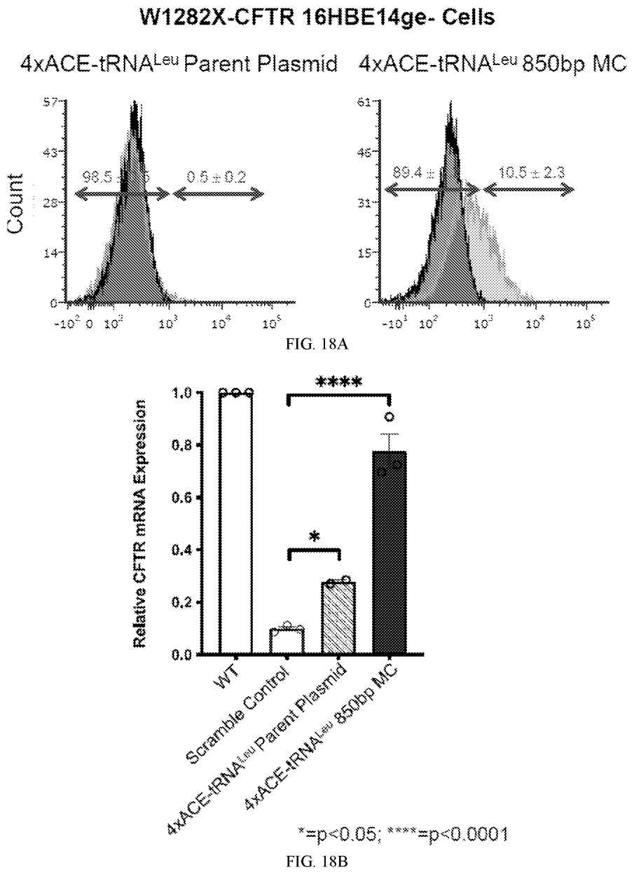

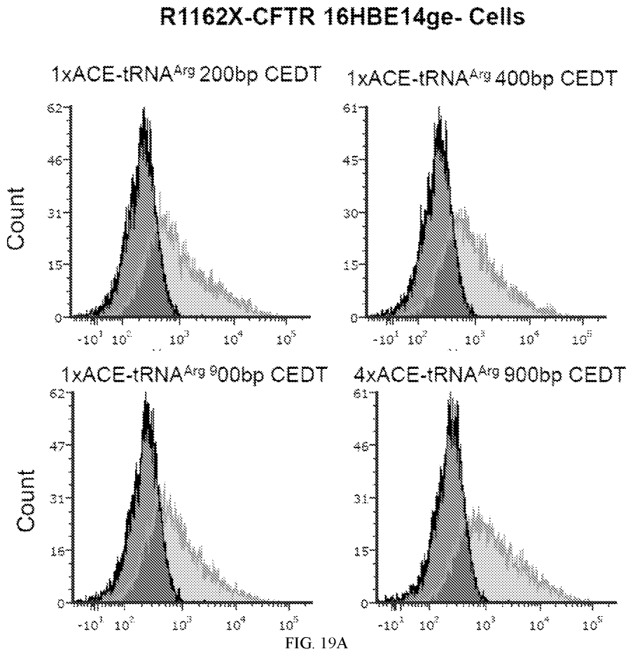

Further assays were carried out using 16HBEge- cells having W1282X-CFTR and the PTC readthrough reporter piggyBac transgene. In these assays, the cells were transfected with a scramble control, plasmids encoding four copies of ACE-tRNA^{Leu} (“4×ACE-tRNA^{Leu} Parent Plasmid”), or MCs of 850 bp encoding four copies of ACE-tRNA^{Leu} (“4×ACE-tRNA^{Leu} 850 bp MC”) by electroporation. The cells were NOT sorted by FACS for mRNA expression analysis, rather the entire population was analyzed. As shown in FIG. 18A, the minicircles supported robust mNeonGreen expression in the majority of cells, indicating that ACE-tRNA expressing minicircles supported PTC suppression to a level that could be quantitated at the protein level. This rescue again was significantly better than the scramble control and the parent plasmid (˜7.0 kb in size). See FIG. 18B.

Similar assays were carried out to show that CEDTs also supported both robust protein expression and rescue of R1162X-CFTR. More specifically, 16HBEge- cells having R1162X-CFTR and the PTC readthrough reporter piggyBac transgene were transfected with a scramble control, plasmids encoding 4×ACE-tRNA^{Arg}, and four CEDTs of different sizes encoding 1× or 4×ACE-tRNA^{Arg} by electroporation. As shown in FIG. 19, CEDTs of 200 bp, 400 bp and 900 bp supported robust CFTR mRNA (FIG. 19B) and mNeonGreen protein expression (FIG. 19A). Their abilities to rescue CFTR mRNA expression and mNeonGreen protein expression were not impacted by the size of the CEDT.

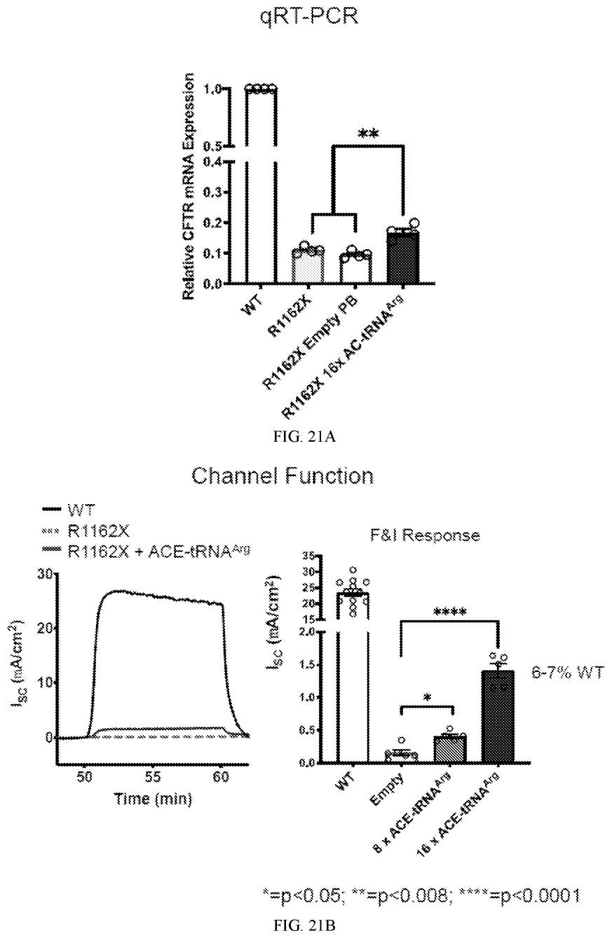

In addition to transfection, which is transient in nature, stable genome integration of ACE-tRNA^{Arg} was also examined. To do that, a PB-donkey system (FIG. 20) was used to generate a vector having multiple copies of ACE-tRNA^{Arg} coding sequences flanked by transposon repeats (TR). This vector was used to generate 16HBEge- cells with ACE-tRNA^{Arg} stably integrated in their genomic DNA. These cells were examined for CFTR mRNA expression and channel function in the manner described above. The results were shown in FIG. 21. It was found that stably expressed ACE-tRNA^{Arg} rescued endogenous CFTR function. Further analysis with quantitative PCR (qPCR) indicated that as few as 16 ACE-tRNA^{Arg} expression cassettes was enough to support robust CFTR function in R1162X 16HBE14ge- cells at about 6-7% of the level for wild type cells.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A closed end, circular, non-viral, and non-plasmid DNA molecule comprising (1) a promoter and (ii) a sequence encoding an anti-codon edited-tRNA (ACE-tRNA).

2. The molecule of claim 1, wherein the molecule is a closed end DNA thread (CEDT) molecule or a minicircle (MC) molecule.

3. The molecule of claim 1, wherein the molecule further comprises one or more elements selected from the group consisting of a DNA nuclear targeting sequence (DTS), a transcription enhancing 5′ leader sequence (TELS), and an ACE-tRNA Barcoding Sequence (ABS).

4. The molecule of claim 3, wherein the DTS comprises a SV40-DTS.

5. The molecule of claim 1, wherein the molecule is free of any bacterial nucleic acid sequence.

6. The molecule of claim 1, wherein the molecule comprises 4 or less CpG dinucleotides or is free of CpG dinucleotide.

7. (canceled)

8. The molecule of claim 1, wherein the molecule is about 200 to about 1,000 bp in size or is about 500 bp in size.

9. (canceled)

10. The molecule of claim 1, wherein the ACE-tRNA comprises a sequence (i) selected from the group consisting of SEQ ID NO: 1-10 or (ii) encoded by one selected from the group consisting of SEQ ID NO: 11-305.

11. The molecule of claim 10, wherein the ACE-tRNA comprises a sequence (i) selected from the group consisting of SEQ ID NO: 1, 4, 5, and 8 or (ii) encoded by one selected from the group consisting of SEQ ID NO: 79 and 94.

12. A pharmaceutical formulation comprising (i) the molecule of claim 1 and (ii) a pharmaceutically acceptable carrier.

13. A method for expressing an ACE-tRNA in a cell, comprising (i) contacting the cell with the molecule of claim 1, and (ii) maintaining the cell under conditions permitting expression of the ACE-tRNA.

14. The method of claim 13, wherein (i) the cell has a mutant nucleic acid comprising one or more premature termination codons (PTCs), (ii) the wild type of the mutant nucleic acid encodes a polypeptide, and (iii) the ACE-tRNA rescues the one or more PTCs and restores expression of the polypeptide.

15. The method of claim 14, wherein the polypeptide is cystic fibrosis transmembrane conductance regulator (CFTR) and the mutant nucleic acid encode a truncated CFTR.

16. The method of claim 15, wherein the mutant nucleic acid has a Trp-to-Stop PTC.

17. The method of claim 16, wherein the ACE-tRNA translates the Trp-to-Stop PTC into a Leu.

18. A host cell comprising the molecule of claim 1.

19. A method of treating a disease associated with a PTC in a subject in need thereof, the method comprising administering to the subject the molecule of claim 1 or a pharmaceutical formulation comprising (i) the molecule and (ii) a pharmaceutically acceptable carrier.

20. The method of claim 19, wherein the disease is selected from the group consisting of cystic fibrosis, Duchenne and Becker muscular dystrophies, retinoblastoma, neurofibromatosis, ataxia-telangiectasia, Tay-Sachs disease, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers, esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes, rickets, Hurler Syndrome, Dravet Syndrome, Spinal Muscular Dystrophy, Usher Syndrome, Aniridia, Choroideremia, Ocular Coloboma, Retinitis pigmentosa, dystrophic epidermolysis bullosa, Pseudoxanthoma elasticum, Alagille Snydrome, Waardenburg-Shah, infantile neuronal ceroid lipofuscinosis, Cystinosis, X-linked nephrogenic diabetes insipidus, McArdle's disease and Polycystic kidney disease.

21. The method of claim 19, wherein the disease is an ocular genetic disease selected from the group consisting of cone dystrophies, Stargardt's disease (STGD1), cone-rod dystrophy, retinitis pigmentosa (RP), increased susceptibility to age-related macular degeneration, Congenital stationary night blindness 2 (CSNB2), Congenital stationary night blindness 1 (CSNB1), Best Disease, VMD, and Leber congenital amaurosis (LCA16).

22. The method of claim 19, wherein the administering is carried out using nanoparticles, electroporation, polyethylenimine (PEI), receptor-targeted polyplexes, liposomes, or hydrodynamic injection.
